(19) United States
(12) Patent Application Publication
(10) Pub. No.: US 2026/0035477 A1
(43) Pub. Date: Feb. 5, 2026

(54) ANTI-PODXL ANTIBODIES FOR INHIBITING CTC CLUSTERING

(71) Applicant: Northwestern University, Evanston, IL (US)

(72) Inventors: Huiping Liu, Evanston, IL (US); Nurmaa Khund Dashzeveg, Evanston, IL (US)

(21) Appl. No.: 18/671,944

(22) Filed: May 22, 2024

Related U.S. Application Data
(60) Provisional application No. 63/503,693, filed on May 22, 2023.

(51) Int. Cl. C07K 16/30; A61K 9/00; A61K 31/337; A61K 31/519; A61K 31/704; A61K 39/00; A61P 35/00; C07K 16/28

(52) U.S. Cl. CPC C07K 16/30; A61K 9/0019; A61K 31/337; A61K 31/519; A61K 31/704; A61P 35/00; C07K 16/2818; C07K 16/2827; A61K 2039/505; A61K 2039/54; A61K 2039/545; C07K 2317/76

(57) ABSTRACT

The present disclosure provides methods for reducing cancer metastasis and enhancing chemotherapeutic efficacy using anti-PODXL neutralizing antibodies.

### CROSS-REFERENCE TO RELATED APPLICATIONS

This application claims the benefit of and priority to U.S. Provisional Application No. 63/503,693 filed on May 22, 2023, the content of which is incorporated by reference in its entirety.

### STATEMENT REGARDING FEDERALLY SPONSORED RESEARCH

This invention was made with government support under CA245699 awarded by the National Institutes of Health and W81XWH2010679 awarded by the Department of Defense, Department of the Army, U.S. Army Medical Research and Materiel Command. The government has certain rights in the invention.

### REFERENCE TO AN ELECTRONIC SEQUENCE LISTING

The contents of the electronic sequence listing (702581.02510.xml; Size: 7,477 bytes; and Date of Creation: May 22, 2024) is herein incorporated by reference in its entirety.

### BACKGROUND

Cancer metastasis is mediated by circulating tumor cells and accounts for 90% of cancer deaths. Chemotherapy resistance and distant metastasis are often connected as main obstacles in cancer treatment. Most circulating tumor cells (CTCs) are detected as single cells, whereas a small proportion of CTCs in multicellular clusters possess 20-100 times higher metastatic propensity than singles. Therefore, strategies for targeting these CTC clusters to prevent or reduce metastasis are of interest.

### SUMMARY

In a first aspect, method for reducing metastasis of a cancer in a subject is provided herein, the method comprising administering to the subject: a therapeutically effective amount of an anti-podocalyxin-like protein 1 (PODXL) neutralizing antibody or an antigen binding fragment thereof; and one or more anti-cancer agents.

The anti-PODXL neutralizing antibody or antigen binding fragment thereof may be administered intravenously. The anti-PODXL neutralizing antibody or antigen binding fragment thereof may be at a concentration of between about 5 and about 20 μg/ml.

The one or more anti-cancer agents comprises one or more chemotherapeutic agents.

In some embodiments, the one or more chemotherapeutic agents comprise at least one of paclitaxel, nano-albumin-bound paclitaxel (PAX-NAB), and doxorubicin. The one or more chemotherapeutic agents may comprise at least one of paclitaxel and nano-albumin-bound paclitaxel (PAX-NAB).

In some embodiments, the one or more anti-cancer agents comprises one or more CDK4/6 inhibitors. The one or more CDK4/6 inhibitors may comprise palbociclib.

In some embodiments, the one or more anti-cancer agents comprises one or more immunotherapeutic agents. The one or more immunotherapeutic agents may comprise at least one of a PD1 inhibitor and a PDL1 inhibitor.

In some embodiments, the one or more anti-cancer agents are administered before the anti-PODXL neutralizing antibody or antigen binding fragment thereof is administered. In some embodiments, the one or more anti-cancer agents are administered concurrently with the anti-PODXL neutralizing antibody or antigen binding fragment thereof. In some embodiments, the one or more anti-cancer agents are administered after the anti-PODXL neutralizing antibody or antigen binding fragment thereof is administered.

The cancer may be a breast cancer. The cancer may be a triple negative breast cancer.

In a second aspect, a method for reducing metastasis of a cancer in a subject is provided herein, the method comprising administering to the subject a therapeutically effective amount of an anti-podocalyxin-like protein 1 (PODXL) neutralizing antibody or an antigen binding fragment thereof.

The subject may have been or will be administered an anticancer agent.

In some embodiments, the one or more anti-cancer agents comprises one or more chemotherapeutic agents. The one or more chemotherapeutic agents comprise at least one of paclitaxel, nano-albumin-bound paclitaxel (PAX-NAB), and doxorubicin.

In a third aspect, a method for reducing circulating tumor cell cluster formation in a subject in need thereof is provided herein, the method comprising administering to the subject a therapeutically effective amount of an anti-podocalyxin-like protein 1 (PODXL) neutralizing antibody or an antigen binding fragment thereof.

### BRIEF DESCRIPTION OF THE DRAWINGS

The patent or patent application file contains at least one drawing in color. Copies of this patent or patent application publication with color drawings will be provided by the Office upon request and payment of the necessary fee.

FIGS. 1A-1N. Chemotherapy correlates with CTC cluster formation and loss of α2,6-SA/ST6GAL1. A, The probability of OS by Kaplan-Meier estimates in the patients with advanced-stage breast cancer, stratified by three alteration patterns of single CTCs (left) or CTC clusters (middle) between baseline (prior to treatment) and the first radiologic evaluation (E1) after 3-month treatment, including (i) decreased CTCs; (ii) stably low or negative CTCs; and (iii) increased or stably high CTCs (singles or clusters). Wilcoxon signed-rank test P<0.0001. CTCs were detected via CellSearch. Right, representative CellSearch images of a single CTC (CK+DAPI+CD45−) at baseline (top row) and a two-cell CTC cluster at E1 (bottom) from a chemo-treated patient (scale bars=10 μm). Dec, decreased; Inc, increased; Neg, negative. B, Dynamic counts of CellSearch-detected CTCs (# events), singles and CTC clusters, at baseline and E1 for each breast cancer patient treated with chemotherapy (light gray lines) and nonchemotherapy options (dark gray lines). Wilcoxon signed rank test for CTC cluster alterations P=0.0158 with chemo (n=47) and P=0.6447 without chemo (n=35); for single CTC alterations P=0.5461 with chemo and P=0.5028 without chemo. C, The probability of OS by Kaplan-Meier estimates in patients with advanced-stage breast cancer with or without chemotherapy (chemo ±, left and right), stratified by the CTC alteration patterns of decreased, negative or stable low, and increased CTC cluster events at E1 compared with baseline (log-rank test P=0.0071 and P<0.0001). CTCs were measured by CellSearch. D, Representative images (left) and quantification (right) of Sambucus Nigra (SNA)-bound α2,6-SA signals in individual CTCs, both singles (S; n=120 cells) and from clusters [C; n=44 tumor cells in the clusters (homotypic and heterotypic clusters of CTCs and CD45+ cells)] of patients with breast cancer (n=6 patients), analyzed by CellSearch (scale bars=10 μm). E, The bar graphs of SNA-high populations within singles (S) and clusters (C) in nonchemo-treated patients (left: −Chemo), chemotreated patients (middle: +Chemo, n=46, P=0.002), and paclitaxel-treated patients (right: +PAX, n=6, P=0.038). F, Schematic of the PAX treatment for PDX-M1 tumors and subsequent analyses of CTCs and lung metastases (mets). One week after orthotopic implantation of PDX-M1 tumor cells into mouse mammary fat pads, mice were treated with PBS or PAX-NAB (PAX; 13.5 mg/kg) once every 3 days via tail vein 10 times. Three days after the last treatment, mice were sacrificed with collections of blood, breast tumors, and lungs for analyses of CTCs, tumors, and metastasis burdens, respectively. G-N, After PBS/PAX treatments shown in F, representative photos of PDX tumors (G; scale bars=1 cm) and quantification of the tumor weight (H), bioluminescence images of dissected lungs ex vivo (I) and quantified lung metastases in total flux (J), counts of single CTCs (K) and CTC clusters (L) in PBS- and PAX-treated mice, and the percentage of SNA-high CTCs in singles vs. clusters in mice treated with PBS (M) and PAX (N). CTCs were analyzed via flow cytometry. P values were calculated by GraphPad (Student t test) unless otherwise indicated. Data, mean±SD.

FIGS. 2A-2L. α2,6-SA and ST6GAL1 inhibit cluster formation and confer sensitivity to PAX. A, Left, flow profile of MDA-MB-231 cells with α2,6-SA-low and α2,6-SA-high populations. Right, representative images of sorted α-2,6-SA-low and α2,6-SA-high cells at 4 hours of clustering (top) and cluster formation curves (cluster size by area) of sorted cells (bottom). Scale bars=60 μm. B, Flow histograms of SNA-binding signals (left), representative cluster images (middle), and cluster formation curves (right) of MDA-MB-231 cells: ST6WT and ST6KO cells transfected with control vectors (−Con), and ST6KO cells transfected with ST6GAL1 overexpression vector (−OE). Scale bars=100 μm. C, Left: cell death (%) of CTC-092 PDX tumor cells ex vivo 24 hours after paclitaxel (PAX) treatments at 0, 25, 50, and 250 μg/mL, measured as DAPI positivity of live cells via flow cytometry (ns, not significant). Right, representative flow profile of DAPI and SNA signals in PAX-treated cells at the high dose of 250 μg/mL. D, Representative flow histograms (left) and SNA-high population (%) within alive CTC-092 cells (right) after 24 hours of PAX treatment at indicated doses (0-250 μg/mL). E, Representative images of CTC clusters formed at 24 hours (left) and the time-course cluster formation curves (right, cluster size) of CTC-092 PDX cells ex vivo upon treatment with PBS and PAX-NAB at 25 g/mL. Scale bar=50 μm. F, Representative flow plots (left) and quantified viability (DAPI exclusion %; right graph) of flow sorting-enriched SNA-high and SNA-low MDA-MB-231 cells (as shown in A) after overnight treatment with PAX at 0 and 25 μg/mL. G, Immunoblots of ST6GAL1, ST3GAL1, FUT3, CD44, and R-actin (loading control) of ST6WT and ST6KO cells at indicated time points (0, 24, and 48 hours) after PAX (25 μg/mL) treatment. Data are representative images of 2 biological replicates. H, Bright-field images of ST6WT and ST6KO cells (left) and their cell viability (%) after treatment with PAX at indicated doses (0-200 μg/mL; right). I, Cell viability of ST6WT and ST6KO cells after indicated time of paclitaxel (PAX) treatment at 25 μg/mL. P values were calculated with the Student t test in GraphPad unless otherwise indicated. Data, mean±SD of 3-5 experimental replicates.

FIGS. 3A-3N. Lack of α2,6-SA and ST6GAL1 is associated with quiescence in CTC clusters. A, Representative CellSearch CTC (CK⁺DAPI⁺CD45⁻) images of two singles and one 5-cell cluster with channels of CK/DAPI merged, DAPI, CD45, and Ki-67 signals, collected from the blood of patients with breast cancer. One single CTC and one CTC within the cluster stained Ki-67 positive. Scale bars=10 μm. B, Ki-67 signal intensity per cell in single CTCs (n=83 cells) and clustered CTCs (n=102 cells, P=0.0005), as well as binary assessment of Ki-67-positive cells (positive threshold at the intensity mean>75) between single CTCs and CTC clusters (P=0.008), as measured on ImageJ. C, Plots of individual CTCs showing a positive association between quantified Ki-67 (proliferative index) and DAPI (DNA content) signal intensities per CTC (n=141, R=0.8618, P<0.00001, calculated using www.socscistatistics.com), as measured on ImageJ. D, Proportion of single and clustered CTC distributions within the cell-cycle phases (G₀-G₁, S, G₂-M) at baseline and E1 after treatment, based on the DAPI (DNA) intensity of each CTC on ImageJ using CellSearch images. The ranges of DAPI mean intensity/cell: G₁/G₀<100, S=100-140, G₂-M>140 (n=1,684 CTCs from 15 patients with breast cancer), P=0.052 (baseline singles vs. baseline clusters), P=8.8E-26 (E1 singles vs. E1 clusters), P=0.935 (baseline singles vs. E1 singles), and P=5.2E-07 (baseline clusters vs. E1 clusters). E, Dot plots of individual CTCs with a positive correlation between SNA signals (α2,6-SA) and DAPI intensity (DNA content) per CTC (n=164 cells, R=0.4052, P<0.00001). F and G, Pearson correlation of the percentage (%) of SNA-high cells and % of Ki-67+ CTCs in M1-PDX tumor-bearing mice (n=20 mice, R=0.6278, P<0.00304 calculated using www.socscistatistics.com; F) and the % of Ki-67+ CTCs in singles vs. clusters (G) in the PBS/PAX-treated PDX-M1 model shown in FIG. 1F-N. H-J, Gene set enrichment analysis of RNAseq of ST6WT vs. ST6KO MDA-MB-231 cells. H, The most downregulated pathways are dark gray, and the most upregulated pathways are light gray. I, The enrichment plots for downregulated pathways include E2F targets, G2-M checkpoint, and MYC targets (V1 and V2). J, The upregulated pathways include interferon response sets, epithelial-to-mesenchymal transition, and inflammatory response genes. K, Cell confluency curves of two clones of ST6WT (1 and 2, ST6WT-2 top curve, ST6WT-1, second curve down) vs. two clones of ST6KO (1 and 2, ST6KO-1, third curve down, ST6KO-2, fourth curve down) cells over 120 hours Data, mean±SD of 12 experimental replicates. L, The relative proportion (%) of α2,6-SA-low and α2,6-SA-high populations in each of the cell-cycle phases of ST6WT cells. M and N, Representative flow profiles (M) and quantification of cell-cycle phases (N) of ST6WT vs. ST6KO cells. Cellular DNA content is measured with Hoechst 33342, and RNA content is measured with Pyronin Y. Quiescent cells (G0) are distinguished from the G1 phase by a low level of RNA content with the same amount of DNA content. P values were calculated with the Student t test in Excel or GraphPad unless otherwise indicated. Data, mean±SD of 3-5 experimental replicates.

FIGS. 4A-4N. ST6GAL1 inhibits cluster formation and blocks metastatic seeding in PDX models. A, Flow cytometry profiles of α2,6-SA (SNA) of indicated PDX models (M1, M2, and M3). B, Representative images (left) and quantification (right) of cluster size (area) of PDX-M1 (M1) vs. PDX-M2 (M2) tumor cells at 24 hours (scale bars=300 μm). Data are represented as mean±SD of 6 experimental replicates. C, Schematic design to analyze spontaneous metastases of M1 (−5 weeks) and M2 (−8 weeks) PDX tumor models: 5×10⁴ cells were orthotopically injected into the fourth mouse mammary fat pads. Five to 8 weeks after implantation, breast tumor weight and lung metastases (mets) were measured. D, Top: images of dissected tumors (scale bar=1 cm) and lung metastases (bioluminescence) of M1 and M2 PDX models. Bottom: quantified tumor weight (g) and lung metastasis (bioluminescence imaging total flux). E, Flow cytometry profiles of α2,6-SA levels in ST6WT and ST6KO PDX-M1/M2 models. F, Flow cytometry profile of α2,6-SA levels (left) and cluster formation curves (right) in ST6WT and ST60E cells derived from M2 PDXs. Curve data are represented as mean±SD of 12 experimental replicates. G, Experimental illustration of tail-vein injection of ST6KO (KO) with appropriate ST6WT (WT) controls of PDX-M1 and PDX-M2 models; 1×10⁵ cells/mouse were injected via tail vein, and lung localization signals were measured 24 hours and/or 5 weeks after injection. H, Representative images (left) and quantification (right) of lung metastatic seedings of ST6WT vs. ST6KO M1 and M2 models 24 hours after injection. Lung total flux of cellular bioluminescence signals was imaged prior to and after injections. I, Lung bioluminescence images (left) and quantified lung metastases (photons/s; right) 5 weeks after tail-vein injection of ST6WT and ST6KO cells. J, Schematic illustration of orthotopic implantations of ST6WT and ST6KO PDX-M1 into the fourth mouse mammary fat pads. Two batches of ST6WT (1.2×10³ cells/injection) and ST6KO (5×10³ cells/injection) cells were injected, and tumors were harvested when reaching ˜0.5 g or 1.0 g. K, Quantified tumor weight for both ST6WT and ST6KO M1 tumors collected at various time points. L, Representative bioluminescence images (left) and quantified lung metastasis (right) of ST6WT and ST6KO PDX-M1 models at 0.5 g and 1.0 g, respectively. Data, mean±SD of 8 experimental replicates. M, Schematic of orthotopic implantations of ST6WT (6×10⁴ cells) and ST60E (2×10⁴ cells) PDX-M2 cells per mammary fat pad, photo of primary breast tumors, and tumor weight comparison between two groups to assess the lung metastases. N, Representative bioluminescence images of dissected lungs (left) and the relative lung metastases, presented as total flux of the lung bioluminescence, at 2 months of implantation. P values were calculated with Student t test using GraphPad, and data are represented as mean±SD of 3-5 experimental replicates unless otherwise indicated.

FIGS. 5A-5H. Glycoproteomic analyses reveal novel α2,6-sialylation targets of ST6GAL1. A, Experimental design of glycoproteome profiles of SNA-bound α2,6-SA+ membrane proteins isolated from ST6WT and KO cells. The membrane fractions were isolated, and α2,6-SA-linked proteins were coimmunoprecipitated with SNA-conjugated agarose beads. Purified proteins were loaded to the glycoproteomics analysis. B, The list of top ST6GAL1 target glycoproteins including their α2,6-sialylation sites on the peptide. C, Immunoblots of the ST6GAL1 substrates PODXL, ECE1, CD97, ALCAM1, and ICAM1 with immunoprecipitation (IP) by SNA (α-2,6-SA+ proteins) or agarose controls, and input controls of the membrane fraction (Mem) or whole-cell lysate (WCL) from ST6WT (+) and ST6KO (−) MDA-MB-231 cells. D, Immunoblots of ST6GAL1 target proteins and the loading control β-actin from the lysates of ST6KO MDA-MB-231 cells transfected with the scramble control (scr) and siRNAs for PODXL, ECE1, CD97, ALCAM1, and ICAM1. E and F, Representative images of clusters at 2 hours (E) and cluster formation curves over the time (0-16 hours; F) of ST6KO MDA-MB-231 cells after downregulation of indicated genes. P scrST6KO vs. WT<0.0001, P scrST6KO vs. siPODXL=0.0001, P scrST6KO vs. siECE1=0.0175, P scrST6KO vs. siALCAM1=0.0260, P scrST6KO vs. siCD97=0.0043, and P scrST6KO vs. siICAM1=0.0003; scale bars=100 μm. P values were calculated with the t test using GraphPad. Data are represented as mean±SD of 12 experimental replicates. G, Representative images of PODXL staining on patient CTCs (CD45−CK+DAPI+) via CellSearch (left) and flow cytometry-based quantification (right) of PODXL expression. H, Schematic (left) and quantification (right) of cell-binding analyses (ST6WT/ST6KO) with PODXL and α2,6-desialylated PODXL isolated from ST6WT and ST6KO cells, respectively. P values were calculated with the Student t test using GraphPad, and data are represented as mean±SD of 3-5 experimental replicates unless otherwise indicated.

FIGS. 6A-6N. Targeting PODXL blocks ST6KO- and chemo-associated metastasis. A-C, Bioluminescence images (BLI) and lung metastasis quantifications of mouse imaging (A) and dissected lungs ex vivo (B), and fluorescence images and relative metastatic seeding (disseminated colonies) to the lungs (C) 24 hours after tail-vein injections of ST6WT and ST6KO cells transfected twice with scrambled control (scr) and siPODXL (siP) for gene knockdown. Scale bars=100 μm. D-F, Experimental design (D), as well as representative images and quantification of mouse BLI (E) and lung ex vivo BLI (F) to determine the lung metastases (met) after administration of IgG and anti-PODXL (αP) neutralizing antibody to ST6WT MDA-MB-231 cells [6.5×10⁴ cells/mice were preincubated with αP antibody (7 μg/mice) or isotope control IgG (7 μg/mice) for 1 hour]. Cells were then injected via the tail vein, and bioluminescence imaging was measured after 24 hours of injection (E). Then mice were sacrificed and ex vivo images of the lungs were measured (F). G, Schematic view of experiment for cotreatment of PAX and αP antibody, PDX-M1 tumor cells were pretreated with PAX-NAB (50 μg/mL) or PBS with αP (20 g/mL) or IgG control (20 μg/mL) and incubated for 12 hours at 37° C. Meanwhile, mice were treated with the same combination of drugs (PAX-NAB, 27 mg/kg or PBS, αP or IgG control, 30 μg/mouse) via tail vein 3 hours prior to cell inoculation. Twenty hours and 3 weeks (3 w) after i.v. injection of the cells, lungs were dissected for ex vivo bioluminescence imaging (H, left, 20 hours) and disseminated lung metastasis quantified as total flux in H (right, 20 hours) and I (3 w). J-N, Experimental design of cotreatment of mice bearing orthotropic (OI) PDX-M1 tumors. First, ST6KO M1-PDX cells were injected into a mammary fat pad. Mice were cotreated with PAX-NAB (13.5 mg/kg) or PBS and αP or IgG control (20 μg/mouse) once every 3 days. After 11 times treatments, mice were sacrificed for further analysis. Tumor weight (K), representative images of lung bioluminescence imaging (L) with normalized quantification of lung total flux with tumor weight (M), and the count of CTC clusters (N; left) and single CTCs (right) are shown. P values were calculated with the Student t test using GraphPad. Data, mean±SD of 3-4 biological replicates.

FIGS. 7A-7E. Methods of CTC analysis (CellSearch and flow cytometry) and overall survival of breast cancer patients based on CTCs at the baseline and E1 after treatment. A. Schematic illustration of three methods for CTC analysis, including CellSearch, flow cytometry, and IHC. B. Representative CellSearch images of single CTCs and CTC clusters of 2-5 or more CTCs per cluster, detected from the blood of the patients with stage III-IV breast cancer. C. Flow profile (left panel) with gated singles and clusters, fluorescent images before and after sorting of MDA-MB-231 cells (L2G-labeled) based on SSA and FSC-A channels (middle panels) (scale bar=50 μm), and quantification (%) of multi-cellular clusters (two or more cells) within unsorted, sorted singles, and sorted clusters (right panel). Student's t-test ***p<0.001, ****p<0.0001. D-E. The probability of overall survival (OS) by Kaplan-Meier estimates in advanced-stage breast cancer patients with and without detection of CTC clusters by CellSearch analysis at the baseline prior to new treatment (N=162) (D) and at E1 after treatment for combined groups of chemotherapy (+/−) (N=71) (E).

FIGS. 8A-8E. Glycosylation profiles of CTCs in the patient blood (singles and clusters). A. Diagram of lectin binding carbohydrate structures and respective catalytic enzyme for transfer of N-glycans. B. Percentage (%) of indicated lectin binding (+ or high) CTCs in the singles (S) versus the clusters (C), as flow detected in the blood of patients with advanced stage III-IV breast cancer. N=60 patients for SNA and N=30 patients for MALII, LEL, RCA, PHA-L, ConA, and LTL. Student's t-test via Graphpad ****p≤0.0001, *p≤0.05, and NS=not significant. Data are represented as mean values+/−SD. C-D. The gating strategies in flow cytometry analyses to isolate single and clustered CTCs (C) and indicated lectin^{high} cells (D) in patient blood samples. Fixed cells were first gated with the size channels SSC-A and FSC-A, then gated for DAPI+ cells with DNA, subsequently divided into singles and clusters by FSC-H and FSC-A, both of which were further gated by CD45. CD45⁺ WBCs were used for the positive control of indicated lectin-high cells (D). Single cells are shown in pink color and clusters were presented in blue color (WBCs in darker colors and CTCs in lighter colors). E. The flow profile and cytokeratin (%) in CD45⁺WBCs and CD45⁻CK⁺ CTCs in the blood of a breast cancer patient.

FIGS. 9A-9H. Phenotypic effects of neuraminidase treatment and CD44 depletion on tumor clustering and sialylation. A. Representative flow histogram of α2,6-SA levels (SNA-binding signals) in the tumor cells treated with vehicle control (Veh) and neuramidinase (NA, 50 mU/mL) (left panel) and representative images of aggregated MDA-MB-231 tumor cell clusters after NA treatment at 0-100 mU/mL for 24 h (scale bars=500 μm) (right panels). B. Bar graph of improved tumor cell cluster formation (cluster size) after NA-mediated removal of SA in a dose-dependent manner (0-100 mU/mL). The size of the clusters are measured by ImageJ. Student's t-test P values are calculated using GraphPad and 14 experimental replicates with +/−SD are shown. C. Cluster formation of CD44-wild type (CD44WT) vs -knockout (CD44KO) cells. Cluster formation was measured by incucyte at indicated time points. ****p>0.0001. D. The validation of CD44 expression in CD44WT and CD44KO cells by flow cytometry analysis. E. Flow cytometry profiles of the glycan detected by indicated lectin bindings in CD44WT (cluster forming) and CD44KO (non-cluster forming) cells. The diagram of binding of lectins to carbohydrates is shown in parenthesis which is also shown in Extended FIG. 2A. F. Western blotting with indicated antibody blotting in CD44WT (WT) and CD44KO (KO) cells. G. mRNA expression level of ST6GAL1 in CD44WT after vehicle (veh) transfection and in CD44KO cells after veh and standard form CD44 (CD44s) transfection. ****p>0.0001. H. Cluster formation of CD44WT and CD44KO cells after transfection of scramble siRNA (scr) and siST6GAL1. **p>0.01, ****p>0.0001.

FIGS. 10A-10C. N-glycomic analysis of α2,6-SA peaks in WT and ST6KO MDA-MB-231 cells by LCMS/MS. A-B. N-glycomic analysis of ST6GAL1 wild-type (ST6WT) and knockout (ST6KO) cells. Solid circles highlight α-2,6-sialic acid (α2,6-SA) peaks exclusively in WT cells whereas dashed circles of α-2,3-sialic acid (α2,3-SA) peaks present in both WT and KO cells. The dashed straight lines are depicting the cross-ring fragmentations of monosaccharide (galactose attached to sialic acid) which are leading to signature fragments indicating the linkage of sialic acids. C. Extracted ion chromatogram from LC-MS/MS spectra of tryptic digest of SDS buffer of eluted glycoproteins from ST6WT and ST6KO cells.

FIGS. 11A-11F. Depletion of ST6GAL1 or ST3GAL1 promotes cluster formation and the viability of CTC092 cells after PAX treatment. A. Representative images (left panels) and the size of tumor cluster area of mouse tumor 4T1 cells (St6gal1 WT and KO) after 1 hour aggregation. Scale bar=150 μm, and Student's t-test ***p<0.001. B. Western blotting of ST6Gal1 expression in ST6WT, 3 clones of ST6KO (1, 2, and 3) cells, and the MDA-MB-231 cells transfected with the scrambled siRNAs control (scr) and siST6GAl1 (siST6). C-D. Proportion (%) of large clusters (>11 or 30 cells) formed by ST6 WT/KO cells (C) and the cells with ST6 knockdown by siRNAs (D). Student's t-test *p<0.05 and **** p<0.0001. E. Left panel: large cluster counts within 2 hours (h) of MDA-MB-231 cells transduced with scramble RNA (scr), siRNA of ST6GAL1 (siST6GAL1, siST6), and siRNA of ST3GAL1 (siST3GAL1, siST3). Student's t-test *p<0.05. Right panels display representative images of cell clusters at 0, 1, and 2 h. Scale bar=100 μm. F. Flow profiles (left panels) and quantification (right) of SNA-high DAPI+ cell death (Q2 subset) of CTC-PDX-092 cells after treatment of nano-albumin bound paclitaxel, PAX-Nab) at different concentrations (0-250 μg/mL). The graph shows the quantification of chemo-induced death, SNA+DAPI+ cells (right panel). Student's t-test p values=0.0066, 0.0002, and <0.0001 as indicated.

FIGS. 12A-12E. ST6GAL1 expression and cell sensitivity to therapeutic treatment (paclitaxel, doxorubicin, and palbociclib). A-B. Cell viability (%) of MDA-MB-231 cells after treatments with 0-4 μg/mL doxorubicin (A) and 0-17.5 mol/L palbociclib (B). P values are calculated with Student's t-test by Graphpad. Data are represented as mean of 6 (A) and 3 (B) experimental replicates. C. Immunoblots using indicated antibodies with MDA-MB-231 ST6WT and ST6KO cell lysate prior to (0 h) and post (24 h) treatment with doxorubicin (Dox, 1 μg/mL). D. Immunoblots of MDA-MB-231 ST6WT and ST6KO cells after 24 h treatment of palbociclib at indicated doses. E. Proportion (%) of α2,6-SA-high and -low cells within mixed ST6WT (SNA high) and ST6KO (SNA low) cells at 1:1, after 24 h treatment of PAX (0 and 25 μg/mL) (p<0.0001). P values are calculated with Student's t-test in Graphpad. Data are represented as mean with ±SD of 3 experimental replicates.

FIGS. 13A-13C. Altered pathways in ST 6 GAL 1 KO cells (cell adhesion and E 2 F targets). A. Pathway analysis of altered mRNAs in ST 6 KO MDA MB 231 cells in comparison with ST 6 WT cells, detected by bulk tumor cell RNA sequencing, showing altered pathways of interferon alpha/beta signaling, cell adhesion, negative cell death, and negative growth. B. Immunoblots of altered cell adhesion molecules in ST 6 KO cells (MKP 5 CERCAM, SPERPINE1 and L1CAM). C. Heat map of E2F target genes in ST6WT and ST6KO cells.

FIGS. 14A-14J. ST6GAL1 deficiency slows tumor cell growth and is associated with Kaplan Meier estimates of patient survival. A. Representative flow cytometry profile (left panel) and bar graph (right panel) of MFI for carboxyfluorescein succinimidyl ester (CFSE) staining of ST6WT and ST6KO MDA-MB-231 cells for cell division analysis. B. Representative images (left panels) and bar graph of the colony size (right panel) of ST6WT and ST6KO cells. Colony size were measured using ImageJ. P values are calculated using Student's t-test via Excel. Data are represented as mean values+/−SD. C. Experimental illustration of cell growth-competition with 20% L2G-WT and 80% L2T-KO cells (D-E) or 50% L2TST6WT (WT) and 50% L2G-ST6KO (KO) or were mixed and plated. Cells (%) with respective colors were counted on indicated days using flow cytometry. D-G. Quantification (D and F) and representative fluorescence images (E and G) of mixed ST6WT and ST6KO cells on indicated days. Student's t-test P values are indicated for each mixed pair over the measured days. H. The probability of distant metastasis-free survival (DMFS) in breast cancer (N=50, median cutoff for the patient groups of SNA high and low, with >10 CTCs or ≤10 CTCs of SNA high expression, Log rank P=0.013). I-J. The probability of relapse-free survival (RFS) (I) or overall survival (OS) (J) by Kaplan-Meier plotter in all breast cancer (N=3951, log rank P=0.0027, q=0.0182) and in TNBC (N=255, log rank P=0.019) by mRNA expression (I) and by protein expression in breast cancer bearing patients (N=65, P=0.0096) (J).

FIGS. 15A-15F. Dynamic glycosylation profile in CTCs of PDXs and tumor models. A-B. Schematic of orthotopic PDX-M1 tumors and representative lung images (A) and table (B) of ST6GAL1 positivity (brown) in single CTC (ST6GAL1⁺), CTC cluster (ST6GAL1⁻) within the vasculature in situ (surrounded by non-clear red blood cells [RBCs]) and disseminated tumor cells (DTCs) (mixed ST6GAL1^{+/-}) in the parenchyma. Scale bar=50 μm, nuclear DNA stained in blue. ST6GAL1⁺ cells (%) within single CTCs, CTC clusters, and DTCs and the Student's t-test P values of each comparison between CTCs (singles and clusters) and DTCs, calculated via GraphPad. C. Flow cytometry profiles (left two panels) and quantified bar graph (right panel) of SNA signals (α2,6-SA levels) in PDX-M1 primary tumor cells, decreased in CTCs, and restored in lung metastases. Biological replicates are included in the bar graph, two tailed Student's t-test for two comparisons P=0.04 and 0.03 respectively. D. Heat map of single cell RNA seq transcriptome of eGFP or tdTomato primary tumor cells, CTCs, and lung metastatic cells sorted from TNBC PDX- and MDA-MB-231 tumor-bearing mice ST6GAL1 was detected in a few cells of primary tumor cells and lung mets but was not detected in CTCs. E. The schematic of tail vein injection of sorted α2,6-SA-high and -low MDA-MB-231 cells (L2G or L2T labeled). Mice were sacrificed for lung dissection on Day 1 (D1, 24 h) and Day 9 (D9, 9 days) after intravenous injections. F. Flow profiles of sorted SNA high and low cells on Day 0 (D 0) prior to tail vein injection, representative images of lung BLI (1 h), lung ex vivo BLI (24 h and D 9), and flow profiles of α2,6-SA levels in the tumor cells disseminated to the lungs, and a bar graph of SNA high cells disseminated to the lungs on D9 as measured via flow cytometry.

FIGS. 16A-16J. ST6GAL1 suppresses the seeding of lung metastasis in PDX and tumor models (related to FIG. 4). A. Summary table of PDX (M1 and M2) and MDA-MB-231 orthotopic tumor models, implanted in comparison pairs of ST6WT and ST6KO cells, ST6 high (M1) and ST6 low (M2), or ST6 WT and ST60E (M2) cells at the same ratio (1:1) or different ratios (1:3, 1:4 or more) for various periods of times for tumor growth and analyses of spontaneous metastases to the lungs via bioluminescence imaging (BLI), immunohistochemistry straining of CTCs and disseminated tumor cells (DTCs) within and outside the CD31⁺ vasculature, respectively, in the lung sections of tumor-bearing mice. B. PDX-M1 tumor images (top left panel), quantified weight of tumors (bottom left panel), and spontaneous lung metastases measured via BLI ex vivo (right panel), developed within 8 weeks after orthotopic implantations of 6e+4 tumors cells, at 1:1 ratio for ST6WT and ST6KO (N=4 mice of 8 tumors for each group). C. PDX-M2 tumor images (ST6WT and KO) (top left panel), tumor weight (bottom left panel), and lung metastases via BLI ex vivo (right panel), at two months after orthotopic implantation with the same numbers of cells, or at the ratio of 1:1, into 4th mammary fat pads. The quantification of lung metastasis of ST6WT and ST6KO of PDX-M2 was normalized with tumor weight. Student's t-test *p<0.05. Data are represented as mean values+/−SD. D-F. L2G⁺ or L2T⁺ PDX-M1 tumor images (D top panel), quantified weight of tumors (D bottom panel), and spontaneous lung metastases measured via BLI ex vivo (E, normalized with tumor weight), developed within 8 weeks after orthotopic implantations of ST6WT tumor cells (1.2e+4) and ST6KO cells (5e+4) cells, at 1:4 ratio (N=4 mice of 8 tumors for each group). Total eGFP+ or tdTomato+ CTC counts (left panel) and CTC clusters (right panel) in the blood of each mouse were measured via flow cytometry (F). G. Representative lung images with CD31 IHC (brown) of ST6WT and ST6KO PDX-M1 tumor bearing mice. Scale bar=50 μm, CTCs (clusters) indicated with arrow heads, and DTCs are pointed with arrows. H. Quantification of CTCs (within the CD31+ vasculature) and DTCs (outside the vasculature) in the lungs of ST6WT and ST6KO PDX-M1-bearing mice. The number of CTCs (single and clustered) per lung image area and number of DTCs per image area are presented in ST6WT and ST6KO groups. Indicated Student's t-test P values are calculated by GraphPad. The data are represented with +/−SD of 15 (WT) and 18 (KO) images. I-J. The representative Ki67 IHC images (I) and bar graphs of lung metastases (# of DTCs/lesion, and % of Ki67⁺ cells/lesion) (J) from PDX-M1 ST6WT and ST6KO tumor bearing mice. Nuclei in blue and Ki67 in brown, scale bar=25 or 50 μm as indicated. P values are calculated with Student's t-test using GraphPad. Data are represented as mean+/−SD of 3-4 biological replicates and 17 experimental replicates.

FIGS. 17A-17D. Counts and Ki67 expression analyses of CTCs and DTCs in situ in the mouse lung sections of the mice bearing ST6WT and ST60E PDXs. A-B. Representative lung images with CD31 IHC staining and pointed CTCs (red heads)/DTCs (purple heads) (B), and bar graphs (C) of vascular CTCs, and extravascular DTCs in PDX-M2 models of ST6WT and ST60E after orthotopic implantation at a cellular ratio of 3:1, as shown in FIG. 4M. C-D. The representative Ki67 IHC images (C) and bar graphs of lung metastases (# of DTCs/lesion, and % of Ki67⁺ cells/lesion) (D) from PDX-M1 ST6WT and ST60E tumor bearing mice. Nuclei in blue and Ki67 in brown, scale bar=25 or 50 μm as indicated. P values are calculated with Student's t-test using GraphPad. Data are represented as mean+/−SD of 3-4 biological replicates and 17 experimental replicates.

FIGS. 18A-18L. Depletion of ST6GAL1 promotes transendothelial migration and seeding. A. Schematic of the tumor cell trans-endothelial migration (TEM) assay (left panel) and quantification of ST6WT and ST6KO MDA-MB-231 tumor cells migrated through HUVEC (%). B-F. Illustrations of tail vein injection of ST6WT (L2T)) and ST6KO (L2G) MDA-MB-231 cells separately (B) or in mixture of both cells (D). 5×104 cells were injected. Seeding to the lungs were measured under fluorescence microscope at 24 h after injections. Representative images of metastatic seeds of ST6WT (red) and ST6KO (green) and the quantifications are shown in C, E, and F. In mixture injected mice, homotypic colonies (WT or KO) and heterotypic colonies (WT+KO) of metastasis are quantified (F). Student's t-test P values are calculated via GraphPad (C, F). G-L. Illustrations of orthotopic injections of ST6WT (red, L2T)) and ST6KO (green, L2G) MDA-MB-231 cells separately or in mixture of both cells (J). For both single and mixed populations, 1×10⁴ cells were injected. Representative images of metastasis of ST6WT (red) and ST6KO (green) (H, K) and the quantifications (I, L) in which total, small, and large lesions of metastases in the lungs are counted. Student's t-test, n.s=not significant, ***p<0.001, *p<0.05 by Excel calculation. Data are represented as mean values of 3-5 biological replicates with +/−SD. Scale bars=200 μm.

FIGS. 19A-19B. CD44 is α2,6-sialylated by ST6GAL1. A. Amino acid sequence coverage of CD44 from SNA pull down of ST6WT lysates, detected by LC-MS/MS (SEQ ID NO: 3). B. The validation of CD44 sialylation by co-immunoprecipitation assay. Membrane fractions were isolated from three different clones of ST6WT and ST6KO MDA-MB-231 cells. SNA bound agarose beads were used to pull down SNA-binding peptides.

FIGS. 20A-20F. Down-regulation of ST6GAL1 target genes reduces cluster formation and de-sialylation of targets increases binding affinity. A. Immunoblots of down-regulated ST6GAL1 target proteins (PODXL, ECE1, CD97, ALCAM, and ICAM1) and the loading control j-actin from the lysates of ST6WT MDA-MB-231 cells transfected with siRNAs for PODXL, ECE1, CD97, ALCAM1, and ICAM1. B. Representative images (top left panels, scale bar=100 m), cluster formation curves over time (bottom left panel), and cell counts (top right panel) of ST6WT MDA-MB-231 cells after knockdown of indicated genes by respective siRNAs. scr; scrambled control RNA. scr; scrambled control siRNA (P scrST6WT vs KO<0.0001, P scrST6WT vs siPODXL<0.0001, P scrST6WT vs siECE1=0.0060, P scrST6WT vs siALCAM1=0.3232, P scrST6WT vs siCD97=0.0347, P scrST6WT vs siICAM1=0.0135. P values are calculated with Student's t-test by GraphPad. Data are represented as mean+/−SD of 12 experimental replicates. C. The cluster curve of ST6WT (WT, dark gray circles) and ST6KO (KO, light gray) cells with scrambled siRNA control (scr) and siDSG2 knockdown. D-E. The mRNA expression levels of ST6GAL1 and PODXL, measured by RT-PCR with different primers (E), and immunoblots of protein expression of ST6GAL1 and PODXL (F) in ST6WT and ST6KO cells. F. Improved solid phase binding of recombinant CD44 in homophilic C44-CD44 interactions after neuraminidase treatment. BSA was used as a negative control.

FIGS. 21A-21H. Down-regulation of ST6GAL1 substrates inhibits metastatic seeding. A-F. Metastatic seeding of ST6KO cells (A-C) and ST6WT cells (D-F) after knockdown of the indicated genes after siRNA transfections. Mouse bioluminescence images (BLI) (A and D), lung ex-vivo BLI (B and E) and metastatic lesions under fluorescence microscope (C and F) are shown with the representative images (top panels) and quantifications (bottom panels). Scale bar=100 μm, P values are calculated using Student's t-test via GraphPad. Data are represented as mean+/−SD of 4 biological replicates. G-H. Cell counts of ST6KO cells (G) and the cell viability (%) (H) of MDA-MB-231 cells after two transfections with scrambled control (scr) and each of the indicated siRNAs, siPODXL (siP), siECE1, siCD97, and siICAM1, measured by hematometer counting and DAPI exclusion (flow cytometry), respectively.

FIGS. 22A-22F. anti-PODXL antibody treatment inhibits tumor cluster formation and metastatic seeding (related to FIG. 5). A. Cluster formation curve of ST6WT MDA-MB-231 cells (2×10⁴ cells/well) after anti-PODXL and IgG treatment at indicated doses. B. Cluster formation curves of ST6KO MDA-MB-231 cells (1.5×10⁴ cells/well) after anti-PODXL treatment in comparison with the IgG isotope control. C. Cluster formation assay of PDX-M2 tumor cells after anti-PODXL and IgG treatment. D. Representative flow profiles of the conjugated anti-PODXL-FITC binding to MDA-MB-231 cells after transfections with scrambled siRNA control (scr) and siPODXL knockdown. E-F. Representative images of mouse BLI (E) and the quantifications of BLI in mice (F, left panel) and the lungs ex vivo (F, right panel) after administration of anti-PODXL neutralizing antibody to ST6KO MDA-MB-231 cells. Cells were pre-incubated with anti-PODXL antibody or isotope control IgG. BLI was performed at 1 h and 24 h after tail vein injection. The normalized fold change was calculated as the total flux of images divided by the total flux of injected cells. Significance are calculated with Student's t-test using GraphPad. Data are represented as mean+/−SD of 3-4 experimental replicates.

FIGS. 23A-23E. Anti-PODXL effects on the metastatic seeding of PDX-M1 tumors after PAX-treatment and the gating strategy of flow analysis of CTCs (related to FIG. 5). A. Representative images of lung ex-vivo BLI and bar graph quantification of CTC-PDX 092 tumor cells inoculated via tail vein to NSG mice. Cells were pre-treated with indicated combinations of drugs (PAX (NAB) (50 μg/ml) or PBS with αP or IgG control (20 μg/mL) for 12 hour prior to inoculation. mice were treated with the same combination of drugs (PAX-NAB, 27 mg/kg or PBS, αP or IgG control, 30 μg/mouse) via tail vein 3 h prior to cell inoculation via tail vein (IV). B. Lung ex-vivo BLIs were measured after 3 weeks. Student's t-test P values are calculated by GraphPad. Data are representative of 3 biological replicates and shown with +/−SD. Non-normalized raw bioluminescence signals of PDX-M1 lung metastases after PAX and α-P treatment in FIG. 6M. C. Images of breast tumors in indicated treatment groups. D. Gating strategy of tdTomato⁺Dapi (viable) CTCs in the blood of PDX-M1 tumor (tdTomato labeled) bearing mice. E. Representative images of tdTomato+ CTCs (left panels) and separation of single versus clustered CTCs (right panels) in each group.

### DETAILED DESCRIPTION

Most circulating tumor cells (CTC) are detected as single cells, whereas a small proportion of CTCs in multicellular clusters with stemness properties possess 20- to 100-times higher metastatic propensity than the single cells. Here, the inventors show that CTC dynamics in both singles and clusters in response to therapies predict overall survival for breast cancer. Chemotherapy-evasive CTC clusters are relatively quiescent with a specific loss of ST6GAL1-catalyzed α2,6-sialylation in glycoproteins. Dynamic hyposialylation in CTCs or deficiency of ST6GAL1 promotes cluster formation for metastatic seeding and enables cellular quiescence to evade paclitaxel treatment in breast cancer. Glycoproteomic analysis reveals newly identified protein substrates of ST6GAL1, such as adhesion or stemness markers PODXL, ICAM1, ECE1, ALCAM1, CD97, and CD44, contributing to CTC clustering (aggregation) and metastatic seeding. The inventors demonstrate that neutralizing antibodies against one newly identified contributor, PODXL, inhibit CTC cluster formation and lung metastasis associated with paclitaxel treatment for triple-negative breast cancer.

In a first aspect, provided herein is a method for reducing metastasis of a cancer in a subject, the method comprising administering to the subject a therapeutically effective amount of an anti-PODXL neutralizing antibody or an antigen binding fragment thereof, and one or more anti-cancer agents. The inventors demonstrate that administering an anti-podocalyxin-like protein 1 (PODXL) neutralizing antibody reduces breast cancer cell cluster formation and metastatic seeding of the cancer cells to the lung. Accordingly, the method also encompasses reducing circulating tumor cell cluster formation.

The term “cancer” refers to any condition characterized by uncontrolled and or abnormal cell growth or uncontrolled division of abnormal cells in a part of the body. The cancer may any type of cancer, including but not limited to, breast cancer, colorectal cancer, esophageal cancer, lung cancer, ovarian cancer, pancreatic cancer, prostate cancer, a skin cancer, stomach cancer, and uterine cancer. In exemplary embodiments, the cancer is a breast cancer. In exemplary embodiments, the cancer is triple negative breast cancer. In some instances, the cancer may be characterized as a solid cancer and further by a tumor or lesion formation. Tumors are generally considered an abnormal mass, or growth in a part of a body caused by abnormal growth of tissue. A tumor may be benign or malignant, primary or metastatic. The terms “metastasis” or “secondary tumor” refer to cancer cells that have spread to a secondary site, e.g., outside of the original primary cancer site. Secondary sites include, but are not limited to, for example, the lymphatic system, skin, distant organs (e.g., liver, stomach, pancreas, brain, etc.) and the like and will differ depending on the site of the primary tumor.

As used herein, the terms “reducing”, “to reduce”, “decreasing”, and “decrease” refer to a statistically significant reduction. For the avoidance of doubt, the terms generally refer to at least a 10% decrease in a given parameter, and can encompass at least a 20% decrease, 30% decrease, 40% decrease, 50% decrease, 60% decrease, 70% decrease, 80% decrease, 90% decrease, 95% decrease, 97% decrease, 99% or even a 100% decrease (i.e., the measured parameter is at zero).

PODXL protein is a member of the CD34 sialomucin protein family, and is mainly localized to the plasma membrane and microtubule organizing center of the cell. PODXL is upregulated in a number of cancers and is frequently associated with poor prognosis.

The terms “antibody” or “antibody molecule” are used herein interchangeably and refer to immunoglobulin molecules or other molecules which comprise an antigen binding domain. The term “antibody” or “antibody molecule” as used herein is thus intended to include whole antibodies (e.g., IgG, IgA, IgE, IgM, or IgD), monoclonal antibodies, chimeric antibodies, humanized antibodies, and antibody fragments, including single chain variable fragments (ScFv), single domain antibodies, and antigen-binding fragments, genetically engineered antibodies, among others, as long as the characteristic properties (e.g., ability to bind antigens derived from Kawasaki disease) are retained. The term “antibody fragment” as used herein is intended to include any appropriate antibody fragment that displays antigen binding function, for example, Fab, Fab′, F(ab′)2, scFv, Fv, dsFv, ds-scFv, Fd, mini bodies, monobodies, and multimers thereof and bispecific antibody fragments.

The term, “neutralizing antibody” is an antibody that inhibits or neutralizes the activity of a molecule to which it binds, e.g. PODXL.

The term “antibody” includes “antibody fragments” or “antibody-derived fragments” and “antigen binding fragments” which comprise an antigen binding domain. Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), (see for instance Bird et al., Science 242, 423-426 (1988) and Huston et al., PNAS USA 85, 5879-5883 (1988)). Such single chain antibodies are encompassed within the term antibody unless otherwise noted or clearly indicated by context.

Antibodies can be genetically engineered from the CDRs, VH, VL, and monoclonal antibody sequences described herein into antibodies and antibody fragments by using conventional techniques such as, for example, synthesis by recombinant techniques or chemical synthesis. Techniques for producing antibody fragments are well known and described in the art.

One may wish to engraft one or more CDRs from the monoclonal antibodies described herein into alternate scaffolds. For example, standard molecular biological techniques can be used to transfer the DNA sequences encoding the antibody's CDR(s) to (1) full IgG scaffold of human or other species; (2) a scFv scaffold of human or other species, or (3) other specialty vectors. If the CDR(s) have been transferred to a new scaffold all of the previous modifications described can also be performed. For example, one could consult Biotechnol Genet Eng Rev, 2013, 29:175-86 for a review of useful methods.

The antibodies or antibody fragments can be wholly or partially synthetically produced. Thus, the antibody may be from any appropriate source, for example recombinant sources and/or produced in transgenic animals or transgenic plants. Thus, the antibody molecules can be produced in vitro or in vivo. The antibody or antibody fragment can be made that comprises all or a portion of a heavy chain constant region, such as an IgG1, IgG2, IgG3, IgG4, IgA1, IgA2, IgE, IgM or IgD constant region.

Furthermore, the antibody or antibody fragment can further comprise all or a portion of a kappa light chain constant region or a lambda light chain constant region. All or part of such constant regions may be produced wholly or partially synthetic. Appropriate sequences for such constant regions are well known and documented in the art.

The term “fragment” as used herein refers to fragments of biological relevance (functional fragment), e.g., fragments which can contribute to or enable antigen binding, e.g., form part or all of the antigen binding site or can contribute to the prevention of the antigen interacting with its natural ligands. Fragments in some embodiments comprise a heavy chain variable region (VH domain) and light chain variable region (VL) of the disclosure. In some embodiments, the fragments comprise one or more of the heavy chain complementarity determining regions (CDRHs) of the antibodies or of the VH domains, and one or more of the light chain complementarity determining regions (CDRLs), or VL domains to form the antigen binding site.

The term “complementarity determining regions” or “CDRs,” as used herein, refers to part of the variable chains in immunoglobulins (antibodies) and T cell receptors, generated by B-cells and T-cells respectively, where these molecules bind to their specific antigen. As the most variable parts of the molecules, CDRs are crucial to the diversity of antigen specificities generated by lymphocytes. There are three CDRs (CDR1, CDR2 and CDR3), arranged non-consecutively, on the amino acid sequence of a variable domain of an antigen binding site. Since the antigen binding sites are typically composed of two variable domains (on two different polypeptide chains, heavy and light chain), there are six CDRs for each antigen binding site that can collectively come into contact with the antigen. A single whole antibody molecule has two antigen binding sites and therefore contains twelve CDRs. For further example, sixty CDRs can be found on a pentameric IgM molecule.

Within the variable domain, CDR1 and CDR2 may be found in the variable (V) region of a polypeptide chain, and CDR3 includes some of V, and all of diversity (D, heavy chains only) and joining (J) regions. Since most sequence variation associated with immunoglobulins and T cell receptors is found in the CDRs, these regions are sometimes referred to as hypervariable regions. Among these, CDR3 shows the greatest variability as it is encoded by a recombination of VJ in the case of a light chain region and VDJ in the case of heavy chain regions. The tertiary structure of an antibody is important to analyze and design new antibodies.

The human VH complex is composed of approximately 100 gene segments per haploid genome, including at least 51 functional genes, as judged by successful rearrangement in cloned cDNA. On the basis of nucleic acid sequence homology, the VH genes have been grouped into 6-7 families (VH 1-7). Among the seven families, the VH3 family is the largest. In some aspects, the antibodies disclosed herein are derived from the VH3-74 family or the VH3-33 family or its paralog.

The anti-PODXL neutralizing antibodies described herein may comprise a polypeptide or protein comprising the antigen binding regions of the antibodies described herein, e.g., the CDRs (1-3) of the heavy and light chain that form the antigen binding region.

As used herein, the terms “protein” or “polypeptide” or “peptide” may be used interchangeable to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids. A protein typically comprises a polymer of naturally or non-naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine).

As used herein, the term “administering” refers to dispensing, delivering or applying the substance to the intended subject by any suitable route for delivery, including delivery by either the parenteral/oral route, intramuscular injection, subcutaneous/intradermal injection, intravenous injection, retro-orbital injection, intrathecal administration, buccal administration, transdermal delivery, topical administration, and administration by the intranasal or respiratory tract route. In preferred embodiments, the anti-PODXL antibody is administered intravenously.

As used herein, a “subject”, “patient”, or “individual” refers to an animal, which may be a human or non-human animal, in need of treatment. In particular embodiments, the subject is a human subject. A “subject in need of treatment” may include a subject having a disease, disorder, or condition that may be characterized as a tumor or cancer.

In embodiments, the concentration of the anti-PODXL antibody is between about 5 and about 20 μg/ml, or any concentration or range in between. In exemplary embodiments, the antibody is formulated at about 7 μg/ml.

As used herein, the term “anti-cancer agent” refers to any agent that treats cancer. Exemplary anti-cancer agents include, without limitation, pharmaceuticals, biologics, toxins, fragments of toxins, alkylating agents, enzymes, antibiotics, antimetabolites, antiproliferative agents, chemotherapeutic agents, hormones, neurotransmitters, DNA, RNA, siRNA, oligonucleotides, antisense RNA, aptamers, lectins, compounds that alter cell membrane permeability, photochemical compounds, small molecules, liposomes, micelles, gene therapy vectors, viral vectors, immunological therapeutic constructs, and other drugs. The one or more anti-cancer agents may be effective at reducing growth of a primary tumor, but have little to no effect on metastasis of the primary tumor cells. Administration of the one or more anti-cancer agents may increase metastasis of the primary tumor cells.

The one or more anti-cancer agents may include one or more chemotherapeutic agents. In exemplary embodiments, the chemotherapeutic agents include paclitaxel, non-albumin-bound paclitaxel (PAX-NAB), and doxorubicin.

Other hemotherapeutic agents are known in the art and include, but are not limited to, platinum coordination compounds, topoisomerase inhibitors, antibiotics, antimitotic alkaloids and difluoronucleosides, as described in U.S. Pat. No. 6,630,124. The chemotherapeutic agent may be a platinum coordination compound. The term “platinum coordination compound” refers to any tumor cell growth-inhibiting platinum coordination compound that provides the platinum in the form of an ion. The platinum coordination compound may be a cis-diamminediaquoplatinum (II)-ion; chloro(diethylenetriamine)-platinum(II)chloride; dichloro(ethylenediamine)-platinum(II), diammine(1,1-cyclobutanedicarboxylato) platinum(II) (carboplatin); spiroplatin; iproplatin; diammine(2-ethylmalonato)-platinum(II); ethylenediaminemalonatoplatinum(II); aqua(1,2-diaminodyclohexane)-sulfatoplatinum(II); (1,2-diaminocyclohexane)malonatoplatinum(II); (4-caroxyphthalato)(1,2-diaminocyclohexane)platinum(II); (1,2-diaminocyclohexane)-(isocitrato)platinum(II); (1,2-diaminocyclohexane)cis(pyruvato)platinum(II); (1,2-diaminocyclohexane)oxalatoplatinum(II); ormaplatin; or tetraplatin. Cisplatin may be the platinum coordination compound employed in the compositions and methods of the present invention. Cisplatin is commercially available under the name PLATINOL™ from Bristol Myers-Squibb Corporation and is available as a powder for constitution with water, sterile saline or other suitable vehicle. Other platinum coordination compounds suitable for use in the present invention are known and are available commercially and/or can be prepared by conventional techniques. Cisplatin, or cis-dichlorodiammineplatinum II, has been used successfully for many years as a chemotherapeutic agent in the treatment of various human solid malignant tumors. Other diamino-platinum complexes have also shown efficacy as chemotherapeutic agents in the treatment of various human, solid, malignant tumors. Such diamino-platinum complexes include, but are not limited to, spiroplatinum and carboplatinum. Although cisplatin and other diamino-platinum complexes have been widely used as chemotherapeutic agents in humans, they have had to be delivered at high dosage levels that can lead to toxicity problems such as kidney damage.

The chemotherapeutic agent may be a topoisomerase inhibitor. Topoisomerases are enzymes that are capable of altering DNA topology in eukaryotic cells. They are critical for cellular functions and cell proliferation. Generally, there are two classes of topoisomerases in eukaryotic cells, type I and type II. Topoisomerase I is a monomeric enzyme of approximately 100,000 molecular weight. The enzyme binds to DNA and introduces a transient single-strand break, unwinds the double helix (or allows it to unwind), and subsequently reseals the break before dissociating from the DNA strand. Various topoisomerase inhibitors have recently shown clinical efficacy in the treatment of humans afflicted with ovarian cancer, esophageal cancer or non-small cell lung carcinoma. The topoisomerase inhibitor may be camptothecin or a camptothecin analog. Camptothecin is a water-insoluble, cytotoxic alkaloid produced by Camptotheca accuminata trees indigenous to China and Nothapodytes foetida trees indigenous to India. Camptothecin exhibits tumor cell growth-inhibiting activity against a number of tumor cells. Compounds of the camptothecin analog class are typically specific inhibitors of DNA topoisomerase I. By the term “inhibitor of topoisomerase” is meant any tumor cell growth-inhibiting compound that is structurally related to camptothecin. Compounds of the camptothecin analog class include, but are not limited to; topotecan, irinotecan and 9-amino-camptothecin. The cytotoxic agent may be any tumor cell growth-inhibiting camptothecin analog claimed or described in U.S. Pat. No. 5,004,758; European Patent Application Number 88311366.4 (Publication Number EP 0 321 122); U.S. Pat. No. 4,604,463; European Patent Application Publication Number EP 0 137 145; U.S. Pat. No. 4,473,692; European Patent Application Publication Number EP 0 074 256; U.S. Pat. No. 4,545,880; European Patent Application Publication Number EP 0 074 256; European Patent Application Publication Number EP 0 088 642; Wani et al., J. Med. Chem., 29, 2358-2363 (1986); and Nitta et al., Proc. 14th International Congr. Chemotherapy, Kyoto, 1985, Tokyo Press, Anticancer Section 1, p. 28-30. In particular, the disclosure contemplates a compound called CPT-11. CPT-11 is a camptothecin analog with a 4-(piperidino)-piperidine side chain joined through a carbamate linkage at C-10 of 10-hydroxy-7-ethyl camptothecin. CPT-11 is currently undergoing human clinical trials and is also referred to as irinotecan; Wani et al, J. Med. Chem., 23, 554 (1980); Wani et. al., J. Med. Chem., 30, 1774 (1987); U.S. Pat. No. 4,342,776; European Patent Application Publication Number EP 418 099; U.S. Pat. No. 4,513,138; European Patent Application Publication Number EP 0 074 770; U.S. Pat. No. 4,399,276; European Patent Application Publication Number 0 056 692; the entire disclosure of each of which is hereby incorporated by reference. All of the above-listed compounds of the camptothecin analog class are available commercially and/or can be prepared by conventional techniques including those described in the above-listed references. The topoisomerase inhibitor may be selected from the group consisting of topotecan, irinotecan and 9-aminocamptothecin.

The preparation of numerous compounds of the camptothecin analog class (including pharmaceutically acceptable salts, hydrates, and solvates thereof) as well as the preparation of oral and parenteral pharmaceutical compositions comprising such a compound of the camptothecin analog class and an inert, pharmaceutically acceptable carrier or diluent, is extensively described in U.S. Pat. No. 5,004,758; and European Patent Application Number 88311366.4 (Publication Number EP 0 321 122), the teachings of each of which are incorporated herein by reference in its entirety.

The chemotherapeutic agent may be an antibiotic compound. Suitable antibiotics include but are not limited to, doxorubicin, mitomycin, bleomycin, daunorubicin, and streptozocin. The chemotherapeutic agent may be an antimitotic alkaloid. In general, antimitotic alkaloids can be extracted from Cantharanthus roseus, and have been shown to be efficacious as anticancer chemotherapy agents. A great number of semi-synthetic derivatives have been studied both chemically and pharmacologically (see, O. Van Tellingen et al, Anticancer Research, 12, 1699-1716 (1992)). The antimitotic alkaloids of the present invention include, but are not limited to, vinblastine, vincristine, vindesine, Taxol and vinorelbine. The latter two antimitotic alkaloids are commercially available from Eli Lilly and Company, and Pierre Fabre Laboratories, respectively (see, U.S. Pat. No. 5,620,985). The antimitotic alkaloid may be vinorelbine.

The chemotherapeutic agent may be a difluoronucleoside. 2′-deoxy-2′,2′-difluoronucleosides are known in the art as having antiviral activity. Such compounds are disclosed and taught in U.S. Pat. Nos. 4,526,988 and 4,808,614. European Patent Application Publication 184,365 discloses that these same difluoronucleosides have oncolytic activity. A 2′-deoxy-2′,2′-difluoronucleoside used in the compositions and methods of the disclosure may be 2′-deoxy-2′,2′-difluorocytidine hydrochloride, also known as gemcitabine hydrochloride. Gemcitabine is commercially available or can be synthesized in a multi-step process as disclosed in U.S. Pat. Nos. 4,526,988, 4,808,614 and 5,223,608, the teachings of each of which are incorporated herein by reference in its entirety.

The one or more anti-cancer agents may comprise one or more CDK4/6 inhibitors. In exemplary embodiments, the CDK4/6 inhibitors include palbociclib. Other CDK4/6 inhibitors include, but are not limited to ribociclib, and abemaciclib.

The one or more anti-cancer agents may comprise one or more immunotherapeutic agents. Immunotherapeutic agents may include antibodies that impact the immune system, checkpoint inhibitors, cytokines, etc. Immunotherapeutic agents, include but are not limited to immune checkpoint inhibitors such as PD-1 inhibitors, PD-L1 inhibitors, CTLA-4 inhibitors, and LAG-3 inhibitors.

Some or all of the anti-cancer agents may be administered before, simultaneously with, or after administration of the anti-PODXL antibody.

The anti-PODXL antibody and any of the anti-cancer agents described herein may be formulated in a pharmaceutical composition comprising further comprising a pharmaceutically acceptable carrier. The term “pharmaceutically acceptable carrier” as used herein means a non-toxic, inert liquid filler, diluent, encapsulating material or formulation auxiliary of any type. The pharmaceutical compositions may be formulated for administration by, for example, injection. Therapeutic compositions typically are sterile and stable under the conditions of manufacture and storage. The pharmaceutically acceptable carrier used in the compositions described herein may be a diluent to dilute the ethanol to the proper percentage, such as sterile water, phosphate buffer saline, and the like. Additional suitable pharmaceutically acceptable carriers are known in the art, and include, but are not limited to, diluents, preservatives, solubilizers, emulsifiers, liposomes, nanoparticles and adjuvants, buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate or other carriers, according to the judgment of the formulator. Additionally, pharmaceutically acceptable carriers may be aqueous or non-aqueous solutions, suspensions, and emulsions. Examples of aqueous carriers include isotonic solutions, alcoholic/aqueous solutions, emulsions or suspensions, including saline and buffered media. Techniques, formulations, and pharmaceutically acceptable carriers may generally be found in Alphonso Gennaro, ed., Remington's Pharmaceutical Sciences, 18th Ed., (1990) Mack Publishing Co., Easton, Pa.

As used herein, the terms “treating” and “to treat” mean to alleviate symptoms, eliminate the causation of resultant symptoms either on a temporary or permanent basis, and/or to prevent or slow the appearance or to reverse the progression or severity of resultant symptoms of the named disease or disorder, including reducing, preventing, ameliorating and/or improving the onset of the symptoms or complications, alleviating the symptoms or complications, or reducing or eliminating the disease, condition, or disorder. For example, treating cancer in a subject includes reducing, repressing, delaying or preventing cancer growth, reduction of tumor volume, and/or preventing, repressing, delaying or reducing metastasis of the tumor. Treating cancer in a subject also includes reducing the number of tumor cells within the subject. The term “treatment” can be characterized by at least one of the following: (a) reducing, slowing or inhibiting growth of cancer and cancer cells, including slowing or inhibiting the growth of metastatic cancer cells; (b) preventing further growth of tumors; (c) reducing or preventing metastasis of cancer cells within a subject; and (d) reducing or ameliorating at least one symptom of cancer.

An “effective treatment” refers to treatment producing a beneficial effect, e.g., amelioration of at least one symptom of a tumor or a cancer. A beneficial effect can take the form of an improvement over baseline, i.e., an improvement over a measurement or observation made prior to initiation of therapy according to the method. A beneficial effect can also take the form of reducing, inhibiting or preventing further growth of a tumor or cancer cells; reducing, inhibiting or preventing metastasis or invasiveness of cancer cells; or reducing, alleviating, inhibiting or preventing one or more symptoms of a tumor, or cancer cells or metastasis thereof. Such effective treatment may, e.g., reduce patient pain, reduce the size of a tumor, reduce the size or number of cancer cells, reduce or prevent metastasis of cancer cells, or slow cancer cell growth. An “effective amount” or “therapeutically effective amount” refers to the amount or dose of the treatment, upon single or multiple dose administration to the subject, which provides the desired effect in the subject. Suitably the desired effect may be reducing metastasis of the cancer. An effective amount can be readily determined by those of skill in the art, including an attending diagnostician, by the use of known techniques and by observing results obtained under analogous circumstances. In determining the effective amount or dose of compound administered, a number of factors can be considered by the attending diagnostician, such as: the species of the subject; its size, age, and general health; the severity of the disease or disorder; the response of the individual subject; the particular treatment administered; the mode of administration; the bioavailability characteristics of the preparation administered; the dose regimen selected; the use of concomitant medication; and other relevant circumstances. The treatment methods disclosed herein may comprise administering the anti-PODXL antibody and any of the anti-cancer agents one or more times. For example, for methods of treatment provided herein, an antibody may be administered in a dosage range (per body weight of the individual) that is between about 0.1 mg/kg to about 50 mg/kg, about 0.5 mg/kg to about 20 mg/kg, about 0.5 mg/kg to about 10 mg/kg, about 0.5 mg/kg to about 5 mg/kg, or 0.5 mg/kg to about 1 mg/kg. One skilled in the art can apply known principles and models of drug delivery and pharmacokinetics to ascertain a likely range of dosages to be tested in preclinical and clinical studies for determining a therapeutically effective amount of a composition or combination used in the methods of treatment provided herein.

In a second aspect, provided herein is a method for reducing metastasis of a cancer in a subject, the method comprising administering to the subject a therapeutically effective amount of an anti-podocalyxin-like protein 1 (PODXL) neutralizing antibody or an antigen binding fragment thereof. The subject has been or will be administered an anti-cancer agent. The one or more anti-cancer agents may be effective at reducing growth of a primary tumor, but have little to no effect on metastasis of the primary tumor cells. Administration of the one or more anti-cancer agents may increase metastasis of the primary tumor cells. The subject may have undergone or will undergo surgery to resect the primary tumor.

“Substantial identity” of amino acid sequences means that a polynucleotide or polypeptide comprises a sequence that has at least 85% sequence identity to a reference sequence (SEQ ID NO) using a sequence alignment program; preferably BLAST using standard parameters. A preferred percent identity of polynucleotides and polypeptides can be any integer from 85% to 100%. A preferred percent identity may be 85%, 86%, 87%, 88%, 89%, 90%, 91%, 92%, 93%, 94%, 95%, 96%, 97%, 98%, or 99% to a reference sequence.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus≤10% of the particular term and “substantially” and “significantly” will mean plus or minus>10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1

### Introduction

Distant metastasis is often coupled with therapy evasion and a poor outcome for patients with cancer. Identifying the cellular mechanisms and molecular targets responsible for metastasis remains one of the most challenging frontiers in cancer medicine. Metastasis is seeded at an extremely low efficiency by single circulating tumor cells (CTC) but at a 20- to 100-fold higher efficiency by multicellular CTC clusters with stemness advantages, due in part to the plastic reprogramming, regenerative properties, and advantageous survival of clusters compared with single CTCs (1-5).

Triple-negative breast cancer (TNBC) has a median overall survival of approximately 18 months (6) and is highly metastatic to viscera, such as the lungs (40%; ref. 7), which has been recapitulated in our established patient-derived xenograft (PDX) models (8). Although chemotherapy has been one of the first-line treatments for TNBC to shrink the primary tumor (9), the benefit of adjuvant chemotherapy might be limited and context-dependent (10-12). In phase III clinical trials for advanced or metastatic TNBC, about 55% of the patients might respond to chemotherapy such as paclitaxel (PAX) or nano-albumin-bound paclitaxel (PAX-NAB; ref. 13), with a median progression-free survival of 3 to 5 months (13, 14). In other large breast cancer trials, the addition of paclitaxel or taxol into a primary systemic or adjuvant therapy did not improve the distant disease-free survival or overall survival (OS) even after improving the response of the local disease to treatment (11, 12). In this study, we found that chemotherapy evasion (chemoevasion) is associated with enriched quiescent CTC clusters and possibly decreased survival for patients with breast cancer. As the microtubule inhibitor paclitaxel is used to treat metastatic breast cancer, we focused on determining its effects on CTCs.

Although collective migration and cohesive shedding could contribute to CTC cluster formation (1, 3), tumor cell aggregation (4, 15) provides an alternative mechanism to initiate and enhance tumor cluster formation (16). We previously demonstrated that cell adhesion and stemness glycoproteins on breast tumor-initiating cells, such as CD44, CD81, and ICAM1, drive CTC aggregation and homotypic cluster formation in metastatic TNBC (4, 15, 17). However, the role of glycosylation in CTC aggregation and cluster formation has yet to be fully elucidated.

Catalyzed by glycosyltransferases, glycosylation is one of the most common posttranslational protein modifications that add hierarchical sugar residues (monosaccharides or polysaccharide glycan chains) onto more than 50% of proteins (18, 19). To compare the glycan profiles between single and clustered CTCs in patients, we utilized a spectrum of fluorophore-bound lectins with specific sugar residue-recognizing preferences for quantitative detection of sialic acids (SA), polylactosamine, galactose, complex type N-glycans, man-nose, and fucose via flow cytometry and the FDA-approved CellSearch platform.

In this report, we found that terminal sugar residue α2,6-sialic acid (α2,6-SA) mediated glycoprotein sialylation, which is mainly catalyzed by β-galactoside α2,6-sialyltransferase (ST6GAL1) in human cells, specifically decreased in the CTC clusters surviving therapies. ST6GAL1 is a type II transmembrane protein that catalyzes the addition of α2,6-SA onto terminal glycans of glycoproteins (20). ST6GAL1 is known to regulate multiple hallmarks of cancer, such as promoting cell proliferation (21-24). This work identified a novel function of ST6GAL1, loss of which in CTCs promotes chemoevasion-associated cluster formation and metastatic seeding in TNBC. Furthermore, using advanced glycome mass spectrometry, we systemically analyzed α2,6-sialylated glycoproteins and identified many new substrates of ST6GAL1 related to cell adhesion or cancer stemness, such as PODXL, ICAM1, and CD44, which contribute to CTC aggregation and lung metastasis of TNBC. The evidence highlights new targeting strategies to block tumor cluster-mediated metastatic seeding associated with poor outcomes for TNBC.

### Results

### Chemoevasive CTC Clusters Predict an Unfavorable Survival for Breast Cancers

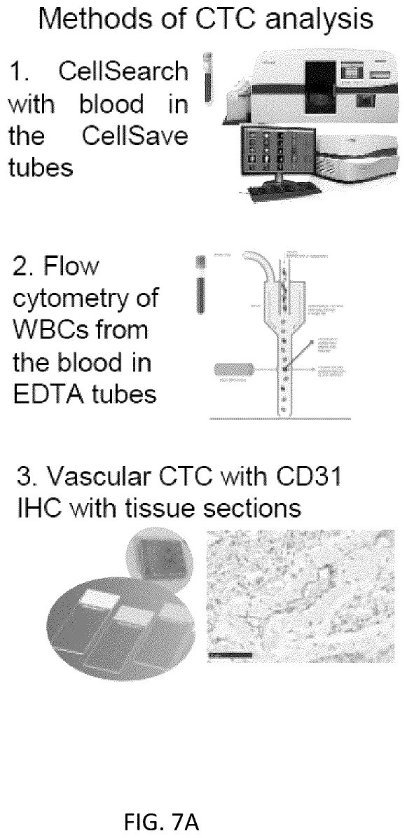

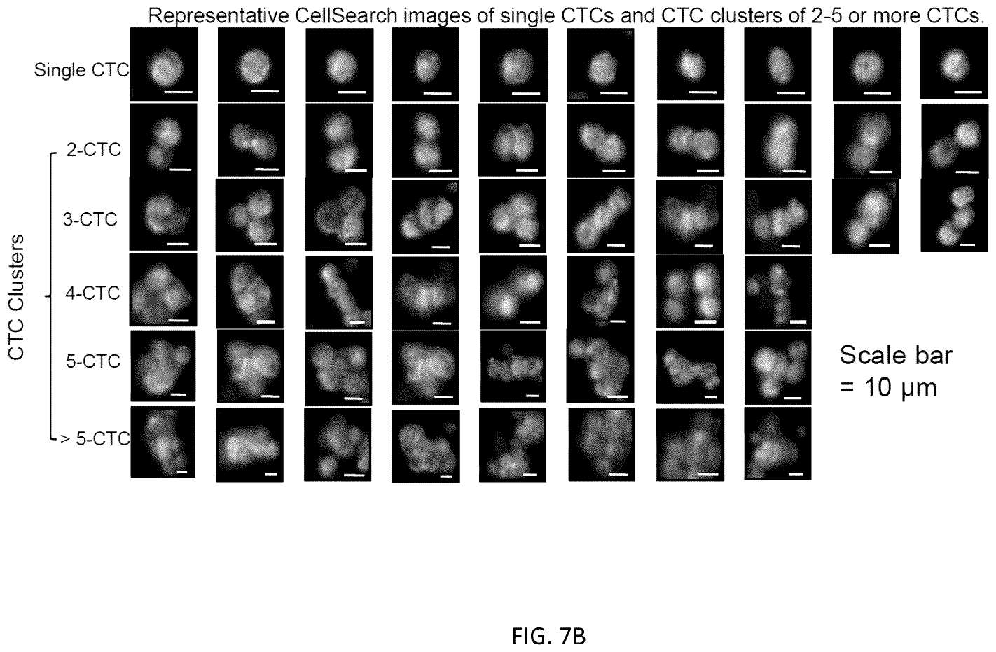

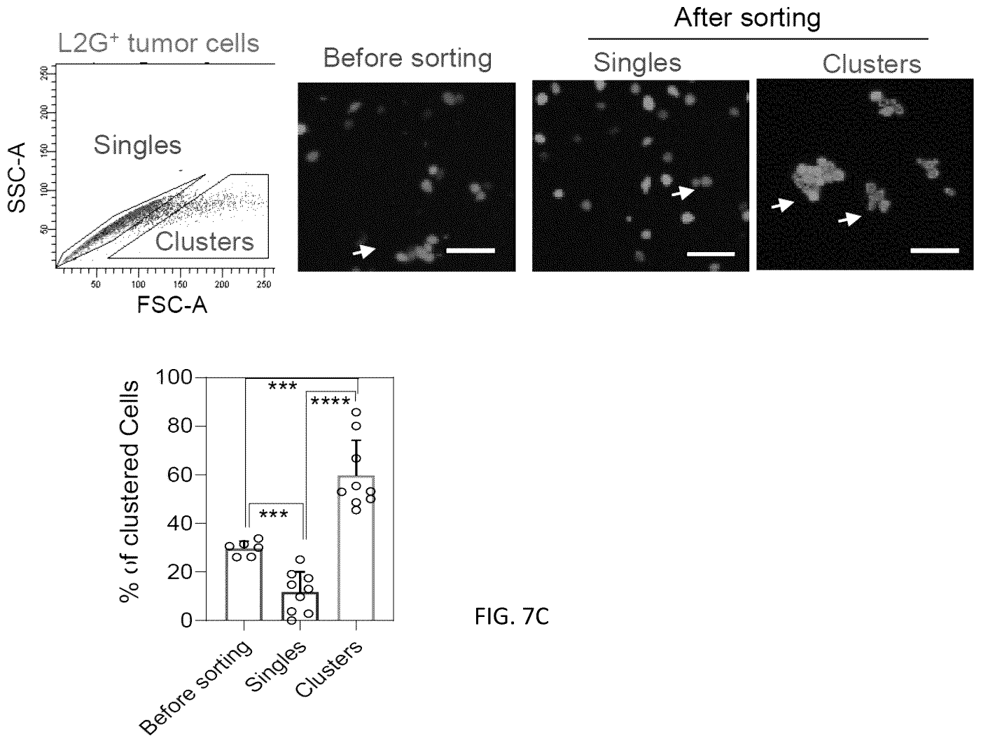

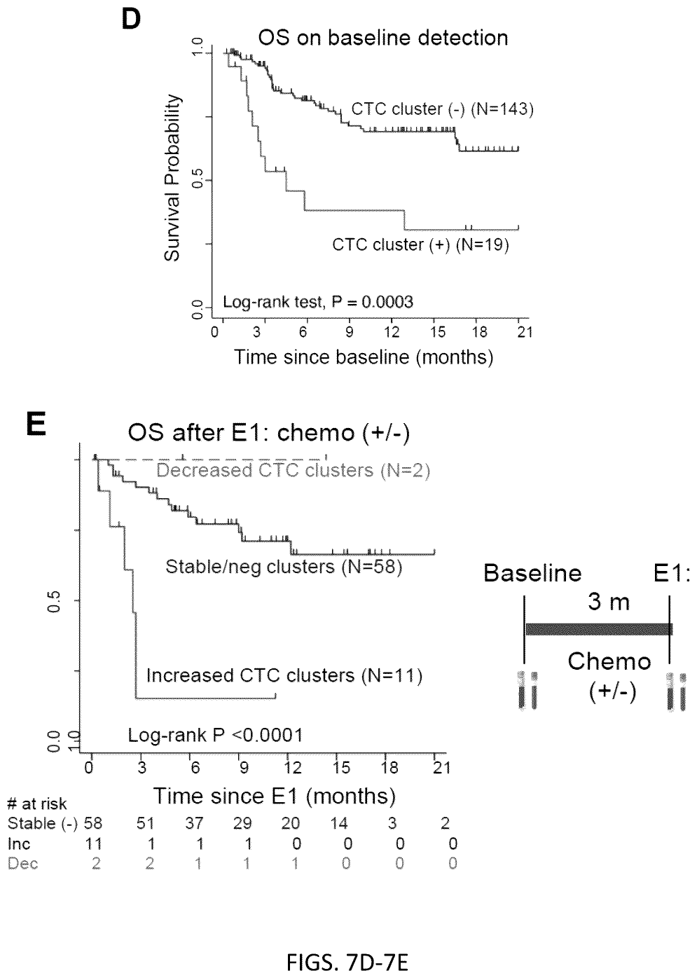

To monitor the longitudinal dynamics of CTCs (singles and clusters) in response to therapy such as chemotherapy or paclitaxel, we established multiple complementary approaches in parallel, including (i) the standard CellSearch-based immunofluorescence staining of EpCAM-enriched CD45⁻ cytokerain (CK)+DAPI+ cells from human blood; (ii) flow cytometry analysis of blood-derived lineage ⁻CD45⁻EpCAM^{+/-} CK^{+/-} cells, with singles and clusters gated on size scatter channels; and (iii) IHC staining of vascular CTCs in tissue sections (FIGS. 7A-7C; refs. 4, 15).

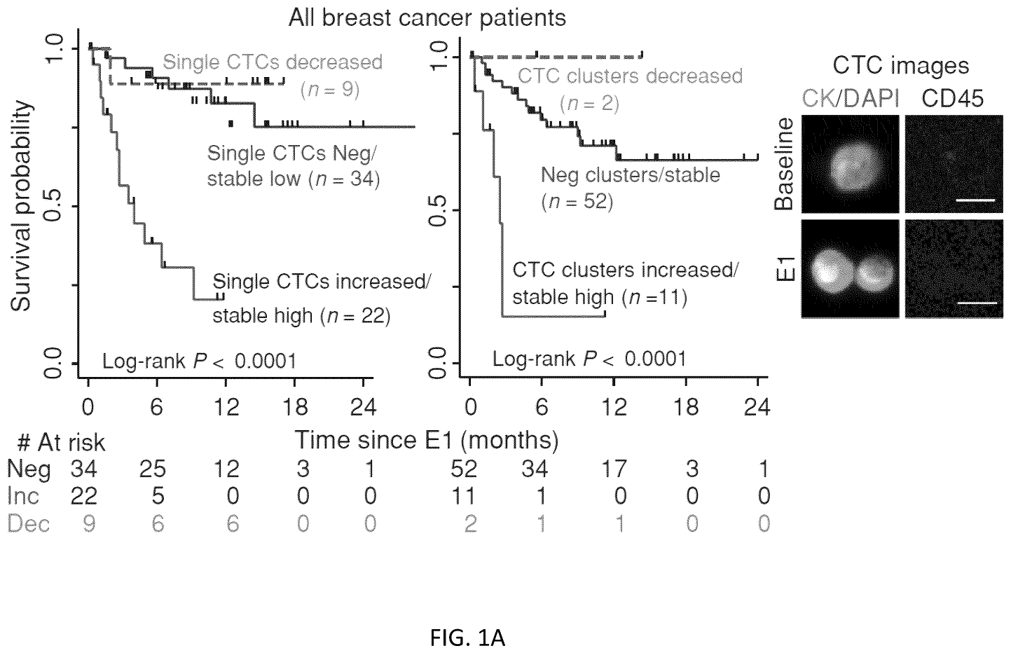

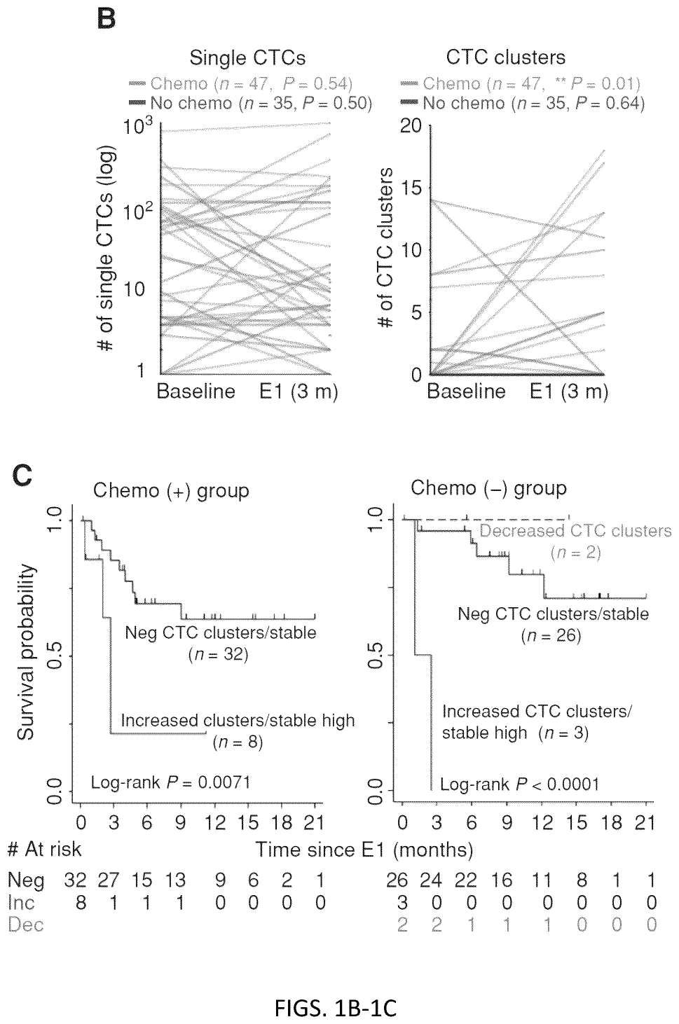

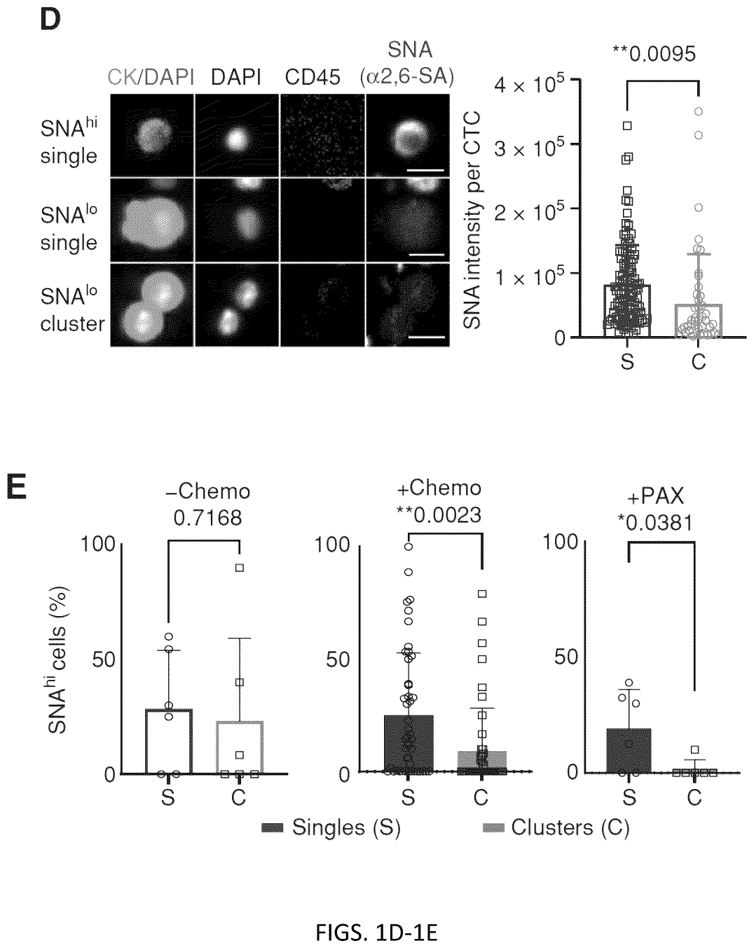

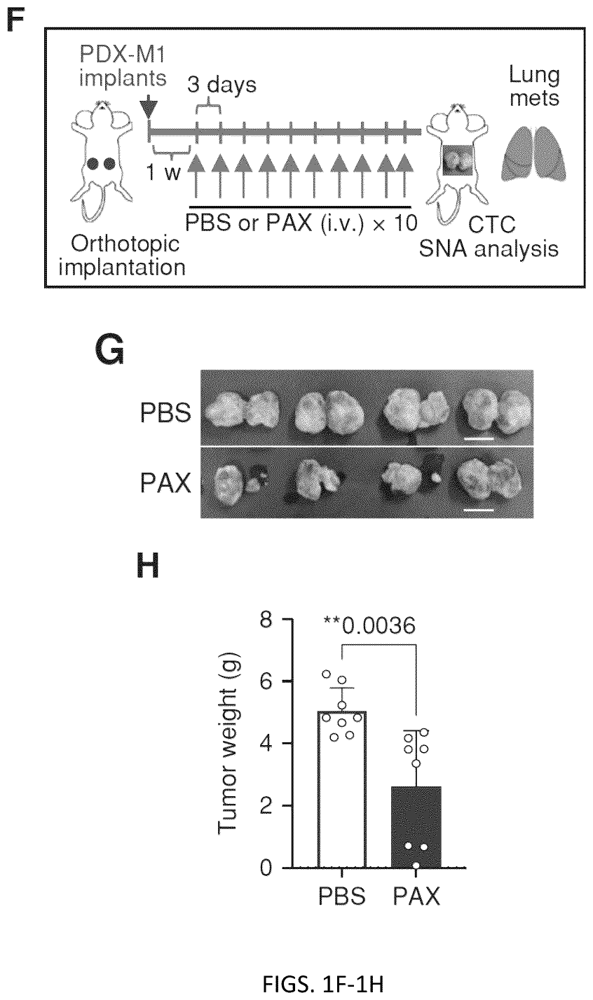

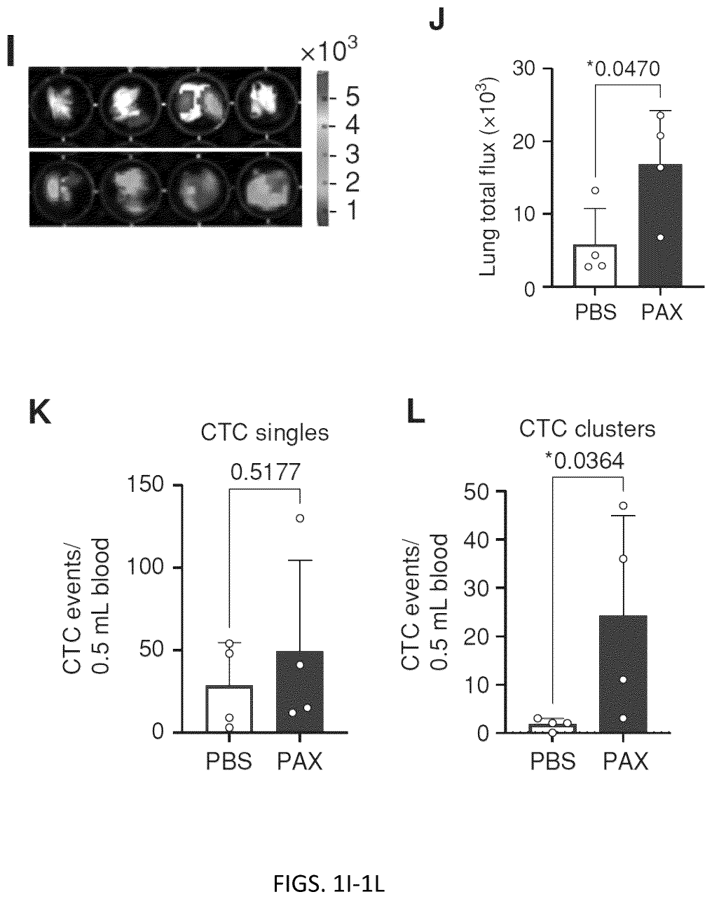

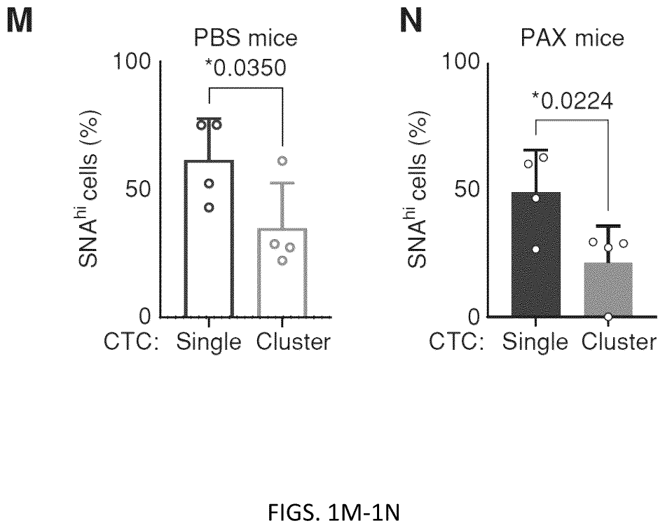

After establishing an institutional review board-approved (IRB) protocol for longitudinal CTC analyses of patients with stage III-IV breast cancer at Northwestern University, we enrolled a cohort of 162 patients for blood collections at baseline prior to a new line of treatment. Around half of the patients were followed up after 3 months of treatment for a second draw of blood and analysis of CTCs (singles and clusters) at the first radiologic evaluation [Evaluation 1 (E1)]. Blood was collected and analyzed for CTC counts (singles and clusters) on CellSearch (FIG. 7B). Consistent with the literature (3, 25) and our previous findings (4, 15), the detection of CTC clusters at baseline is associated with an unfavorable OS (n=162, P=0.0003; FIG. 7D). Among those with follow-up CTC monitoring and survival analysis (n=65), the patients with decreased CTCs (single or clusters) at E1 showed the best OS compared with a moderate survival for those with stably low or negative counts of single CTCs/CTC clusters, and those with increased (or stably high counts) single CTCs/clusters at E1 after therapy showed the worst survival (FIGS. 1A and 7E). Using a multivariable Cox regression model, we found that CTC clusters at E1 is an independent factor predicting an unfavorable OS for patients with breast cancer.

About half of the patients analyzed at E1 had received chemotherapy and the rest nonchemotherapy options. We compared the dynamic patterns of CTCs (singles and clusters) between baseline and E1 among chemotherapy and nonchemotherapy groups. The number of CTC clusters, but not single CTCs, increased significantly at E1 in the patients after chemotherapy (n=47, Wilcoxon signed rank test P=0.0158), whereas mixed fluctuations (without statistical significance) in CTCs (singles or clusters) were observed in the patients who did not receive new chemotherapy (n=35; FIG. 1B). About 20% of the chemo-treated patients (8 out of 40) and ˜10% of nonchemo-treated patients (3 out of 31) showed increased CTC clusters (or stable high) and worse survival in comparison with those with stably low (negative changes) or decreased CTC clusters (FIG. 1C). Decreased CTC clusters were found only in the patients treated with nonchemo approaches (FIG. 1C, right) but not observed in the chemo-treated group (FIG. 1C, left). These data suggest that CTC clusters may confer therapy-resistant or chemoevasive features that have yet to be fully elucidated.

Loss of α2,6-Sialylation in Clustered CTCs and Associated with PAX Treatment

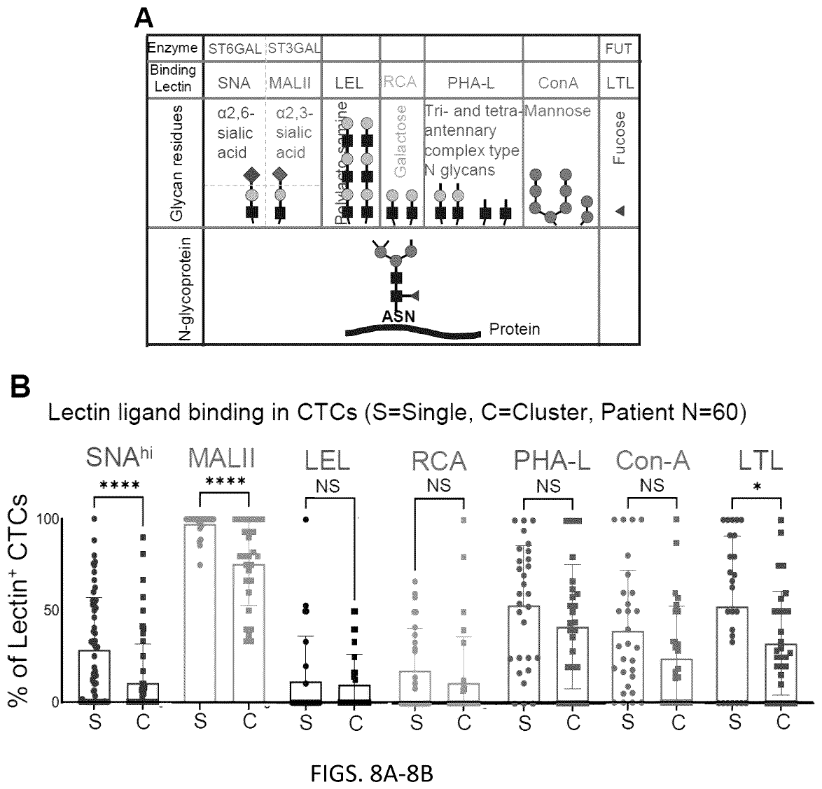

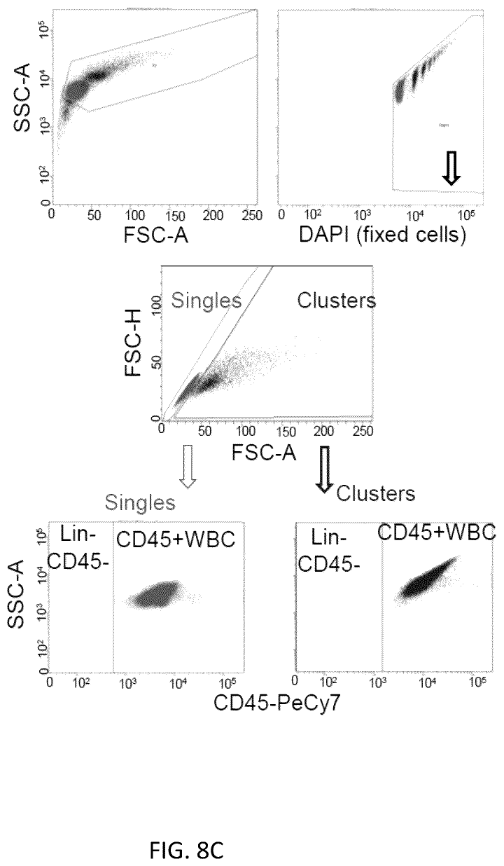

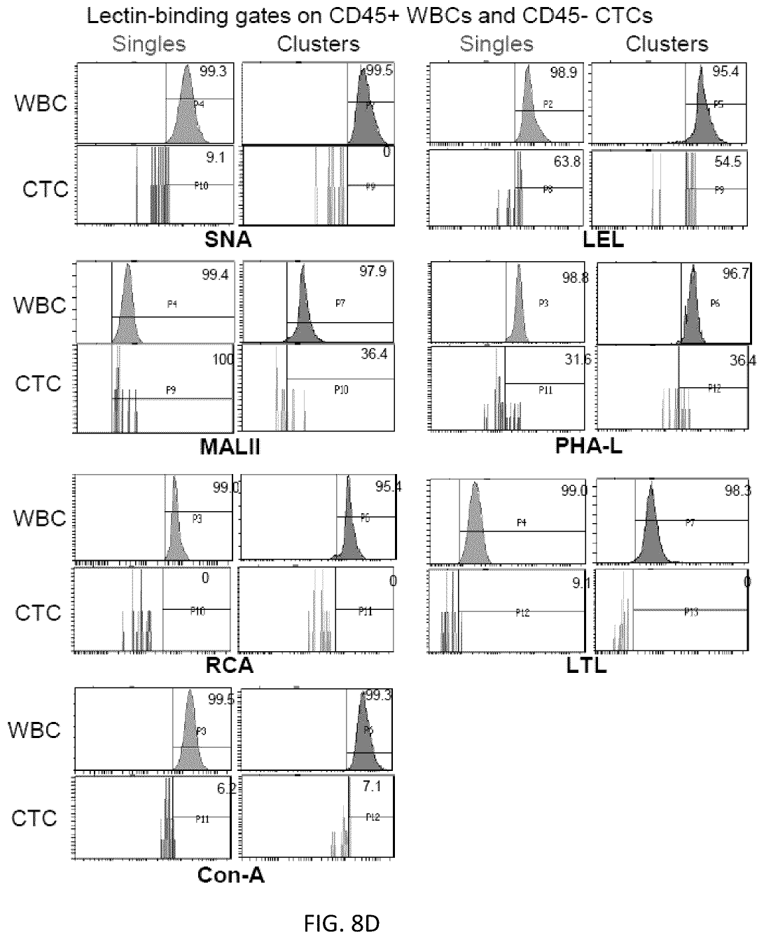

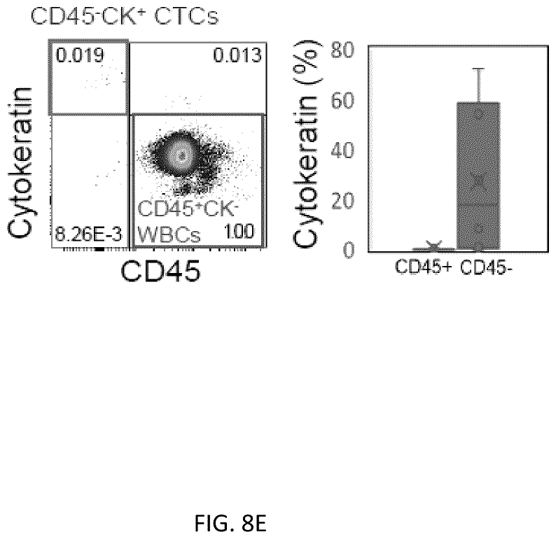

To investigate whether cell-surface glycosylation impacts CTC cluster formation, we first optimized the flow cytometry approach to analyze the glycosylation profiles in EpCAM^{+/-}CK^{+/-} CD45⁻CTCs (clusters and singles) using lectin-based recognition of various carbohydrate residues. These include the terminal sialylation residues α2,3-SA and α2,6-SA [recognized by lectins MAL-II and Sambucus Nigra (SNA), respectively], polylacto-samine (LEL), galactose (RCA), tri- and tetra-antennary complex type N-glycans (PHA-L), mannose (ConA), and fucose (LTL; FIG. 8A). Among all tested glycans, the levels of SNA-bound α2,6-SA had the most dramatic reduction in clustered CTCs along with a moderate decrease of α2,3-SA and fucose levels compared with single CTCs from patients with breast cancer (n=60, P<0.0001), whereas the single and clustered white blood cells (CD45⁺) had comparable levels for tested glycans (FIGS. 8B-8E). Consistently, when analyzed on the CellSearch platform, CK⁺DAPI⁺CD45⁻CTCs in the clusters displayed lower α2,6-SA levels (SNA binding) per cell than the singles (FIG. 1D), confirming the flow analysis data. Moreover, in comparison with single CTCs, α2,6-SA levels specifically decreased in the CTC clusters of chemo-treated and paclitaxel (PAX)-treated patient groups, but not in the nonchemo-treated group (FIG. 1E), implicating an association of hyposialylation with chemotherapy or PAX treatment.

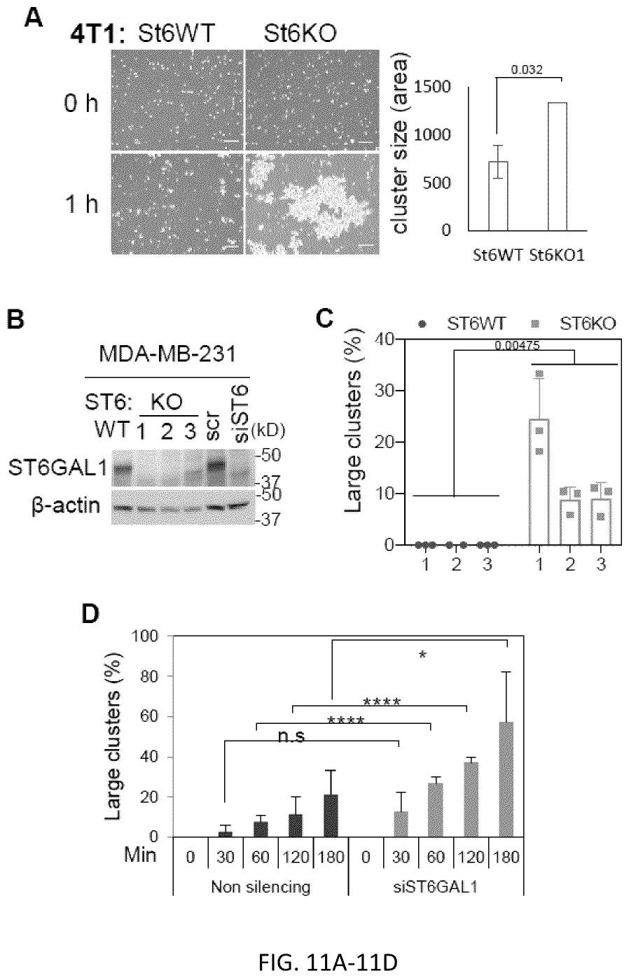

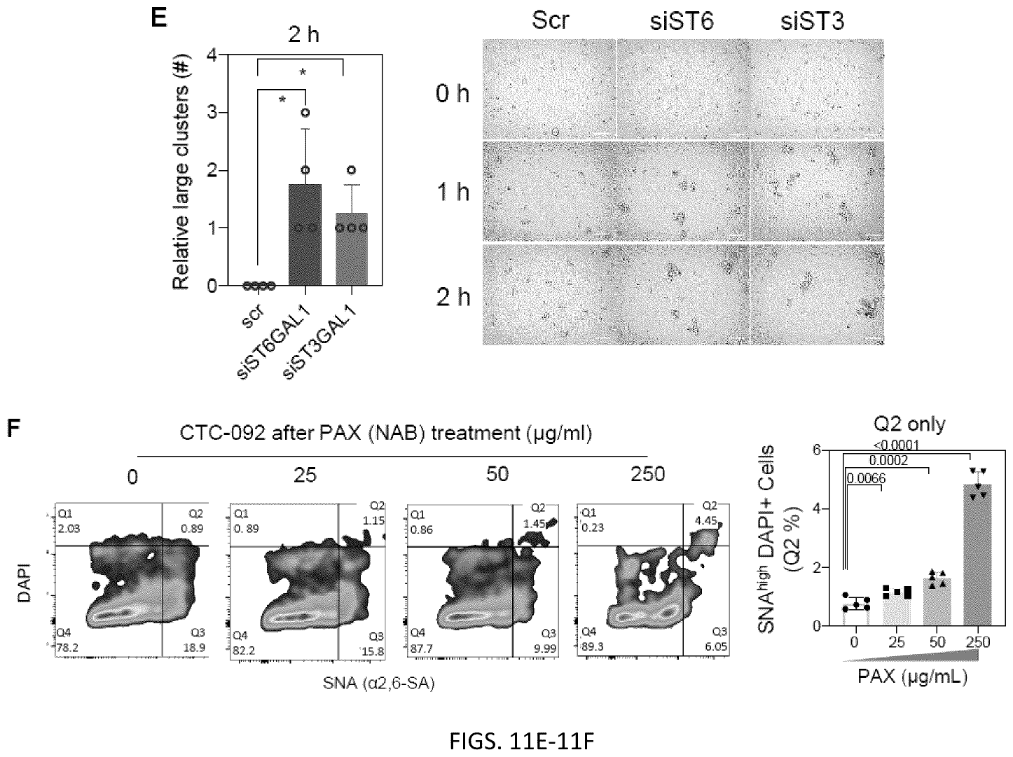

As a proof of concept, we treated mice with PAX following orthotopic implantation of luciferase 2-eGFP (L2G)- or luciferase 2-tdTomato (L2T)-labeled TNBC PDX cells (8) into the fourth mammary fat pads (FIG. 1F). The tumor sizes were slightly reduced in response to PAX treatment (FIGS. 1G and H); however, the spontaneous metastases to the lungs were significantly higher in the PAX-treated group than in the control PBS-treated group (FIGS. 11 and J). Blood analysis of L2G⁺ or L2T⁺ CTC via flow cytometry did not identify significant changes in the total number of single CTCs between the two groups of mice (FIG. 1K), whereas the events of CTC clusters were significantly higher in the PAX-treated group than in the PBS control group (FIG. 1L). CTC clusters displayed lower levels of α2,6-SA sialylation (SNA signals) than single CTCs in both groups (FIGS. 1M and N). These findings suggest that chemoevasive CTC clusters are associated with PAX treatment, enhanced lung metastasis, and loss of α2,6-sialylation.

### Loss of α2,6-SA or ST6GAL1 Promotes CTC Cluster Formation, Leading to the Evasion of PAX Treatment

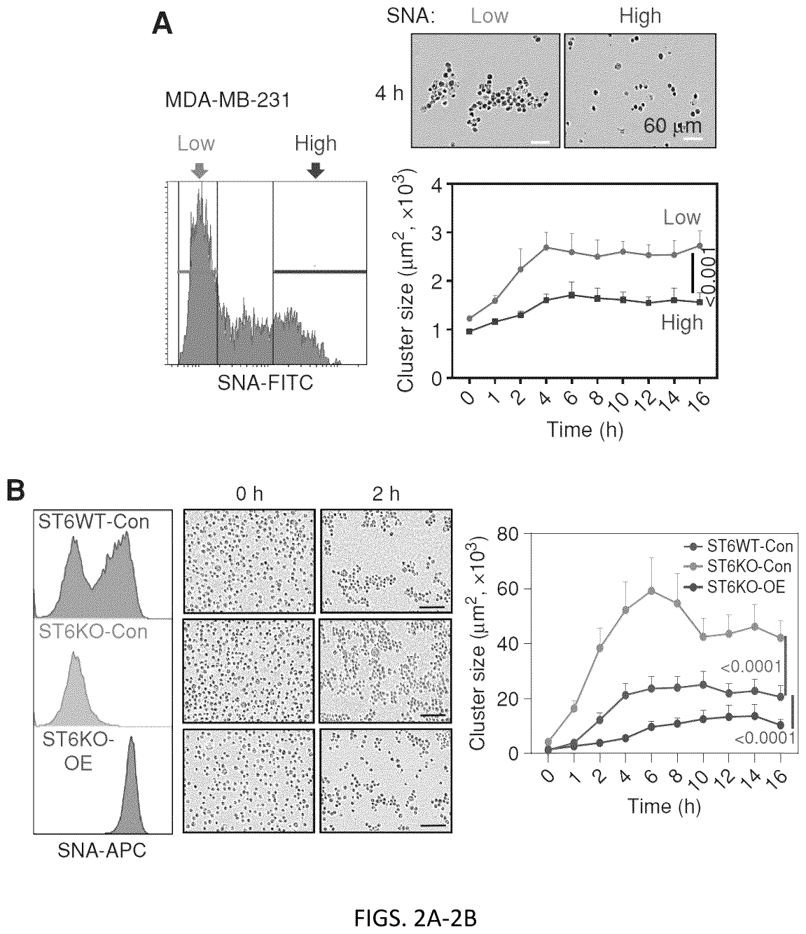

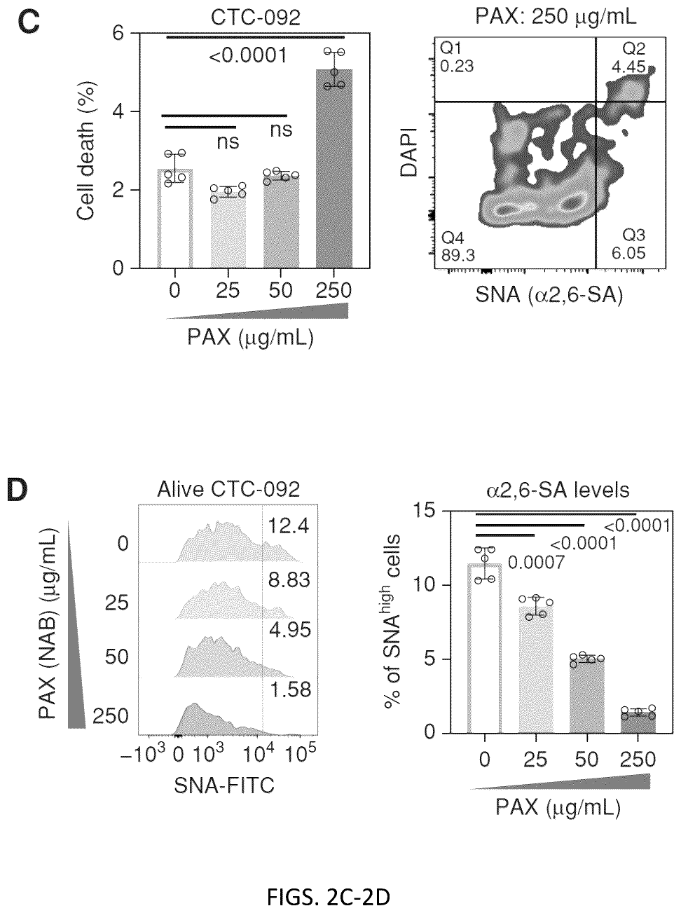

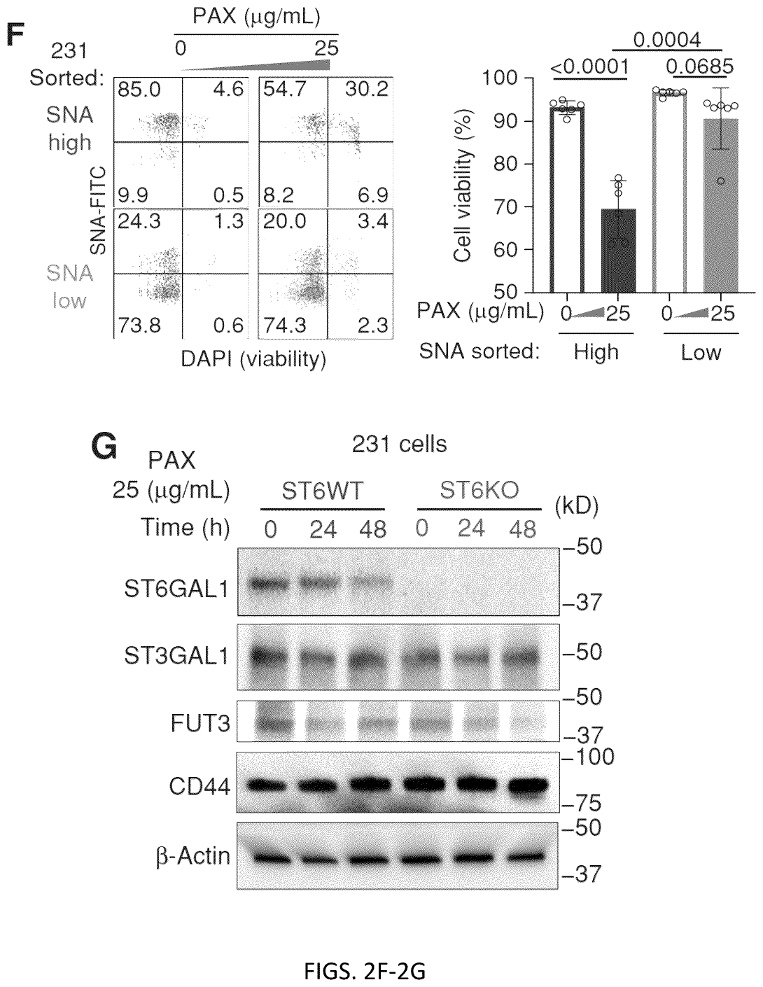

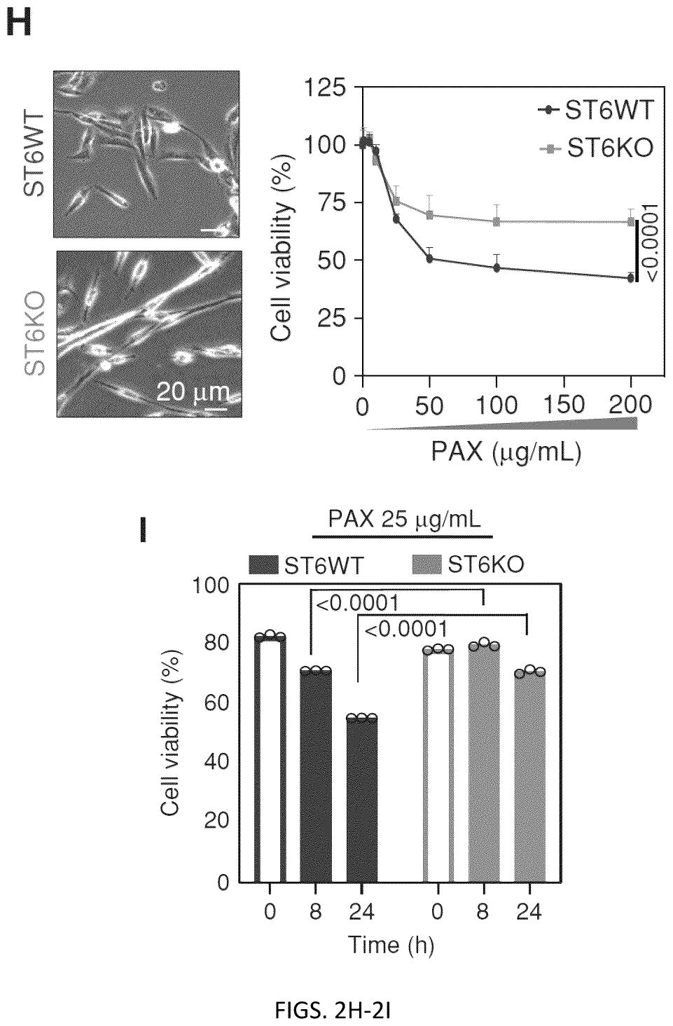

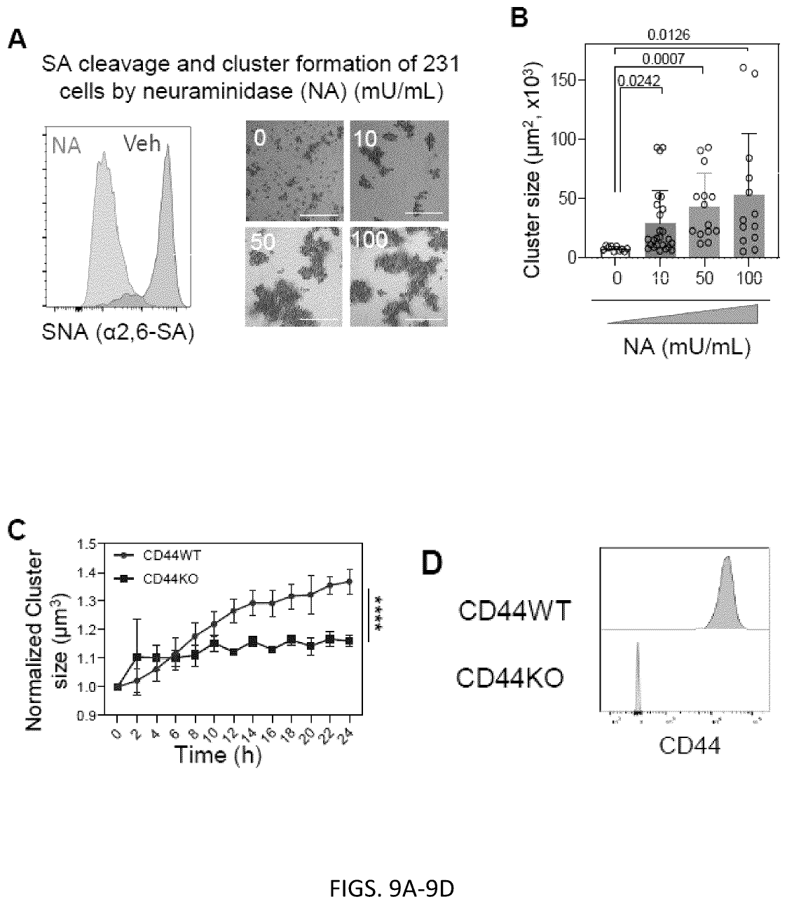

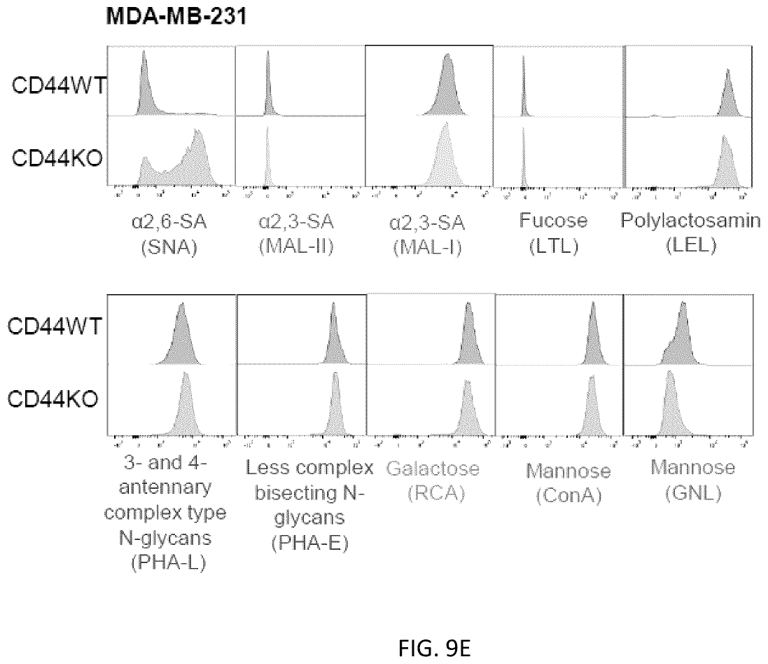

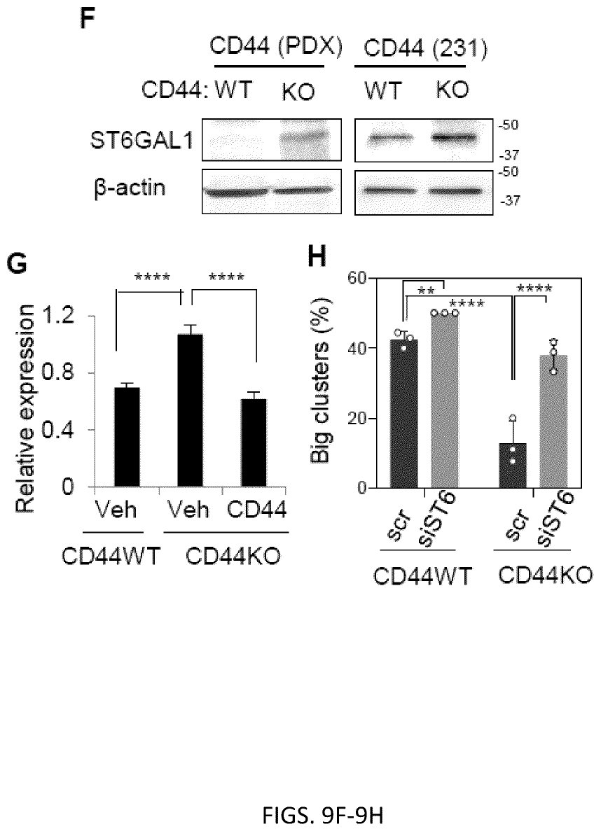

To determine whether α2,6-SA levels and ST6GAL1 influence tumor cell clustering, we first sorted the SNA-high and SNA-low subpopulations from the metastatic MDA-MB-231 cells (FIG. 2A). When seeded onto poly-HEMA-treated plates for cell suspension and cluster formation assessment, the SNA-low cells aggregated into bigger clusters than SNA-high cells (FIG. 2A, right). Moreover, neuraminidase (NA)-mediated cleavage of SA (2,6-SA, α2,3-SA, and α2,8-SA) from the cell surface also drastically promoted tumor cluster formation (FIGS. 9A and 9B). Because CD44 was one of the main adhesion molecules and breast tumor initiation markers mediating CTC cluster formation (4), we compared the glycan profiles in 9CD44 wild-type (WT) and CD44 knockout (KO) cells. CD44KO cells with compromised clustering capabilities increased α-2,6-SA levels and ST6GAL1 expression without altering other tested glycan residues (FIGS. 9C-9F). CD44 overexpression inhibited ST6GAL1 expression in CD44KO cells and siST6GAL1 knockdown partially rescued the tumor cell formation of CD44KO cells (FIGS. 9G and 9H).

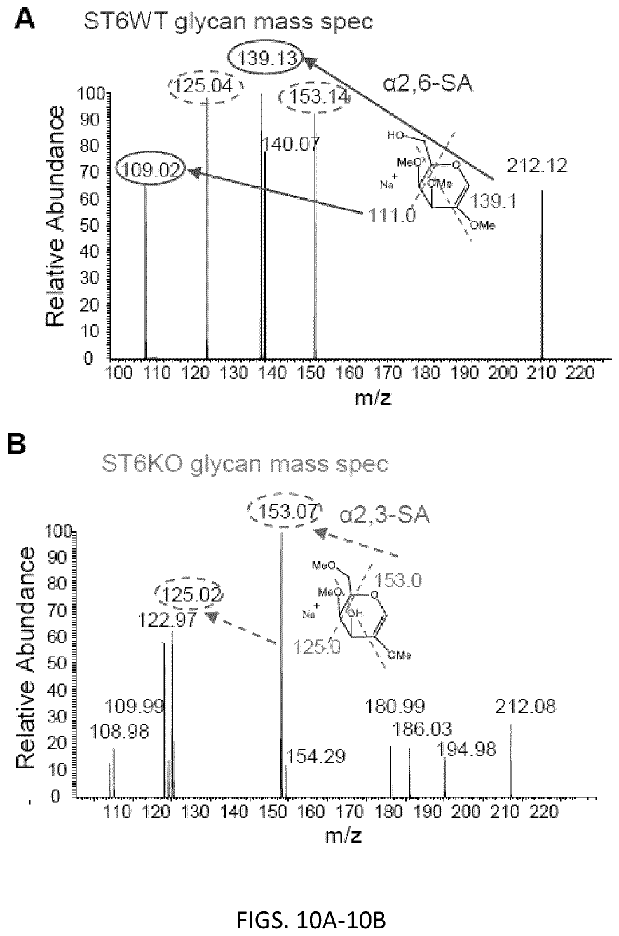

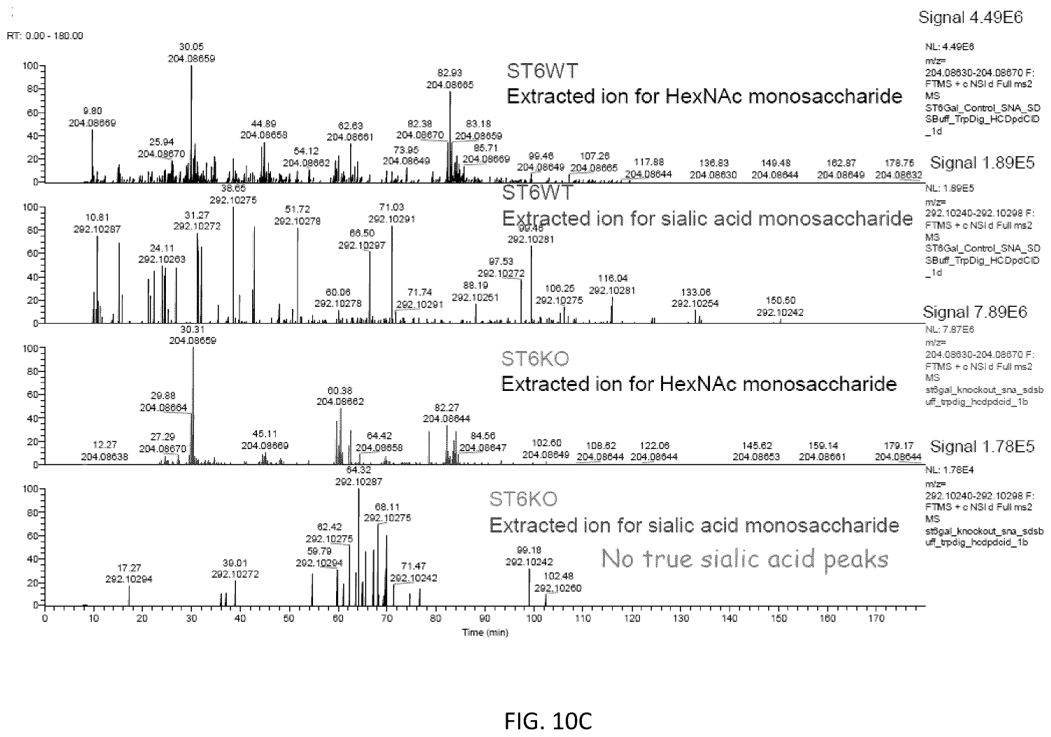

We continued to determine the regulatory effects of ST6GAL1 gene modulation (depletion and overexpression) on human and mouse tumor cell clustering. Both human ST6GAL1 KO (ST6KO) tumor cells (MDA-MB-231 and PDXs) and mouse St6gal1 KO (St6KO) tumor cells (4T1) were generated via CRISPR-Cas9 and specific gRNAs. Using glycoproteomic mass spectrometry, we observed a complete depletion of the peaks of α2,6-SA on glycoproteins, whereas α2,3-SA peaks remained in ST6KO MDA-MB-231 cells in comparison with the WT cells (FIGS. 10A and 10B), consistent with the loss of sialic acid peaks in the LC-MS/MS spectra of the tryptic digest for extracted ion HexNAc and SA monosaccharide profiles, as well as loss of SNA binding to these cells in flow cytometry profiling (FIG. 2B, left; FIG. 10C), suggesting a dominant role of ST6GAL1 in α2,6-SA of breast tumor cells.

Consistently, both ST6KO in human tumor cells and St6KO in mouse 4T1 tumor cells promoted the cluster formation of tumor cells in suspension compared with their WT controls (FIGS. 2B and 11A FIG. S5A). When ST6GAL1 was overexpressed (OE) in the ST6KO cells, the α2,6-SA levels were rescued with inhibited cluster formation (FIG. 2B). Similarly, the ST6GAL1 depletion-enhanced cluster formation was repeatedly observed in multiple ST6KO clones of MDA-MB-231 cells, as well as the cells with siRNA-mediated knockdown of ST6GAL1 (FIGS. 11B-11E). These results suggest that CTC aggregation-based cluster formation is negatively associated with and inhibited by cell-surface α2,6-SA levels, as well as ST6GAL1.

We then investigated the effects of chemotherapy such as PAX on the levels of surface α2,6-SA and cluster formation of patient CTC-092-derived xenograft tumor cells (PDX; ref. 26) and MDA-MB-231 cells. Clinically used PAX in nano-albumin-bound version (PAX-NAB) at 25 to 50 μg/mL induced minimal cell death in CTC-092 cells ex vivo, with a small subset of SNA-high cells showing a selective vulnerability (<5%) to the high-dose treatment at 250 μg/mL (FIGS. 2C and 11F). As a result, PAX treatment decreased α2,6-SA levels (SNA binding) in a dose-dependent manner (FIG. 2D) and promoted cluster formation of chemo-resistant CTC-092 cells at a low dose of 25 μg/mL (FIG. 2E). Next, we treated sorted SNA-high and SNA-low cells for PAX treatment and assessed cell viability. Although 35% of the cells in SNA-high populations died in response to PAX treatment, minimal cell death was observed in SNA-low populations (FIG. 2F), suggesting that α2,6-SA deficiency is associated with resistance and/or evasion to PAX treatment. Notably, PAX treatment selectively decreased ST6GAL1 protein expression over time without altering ST3GAL1 levels (FIG. 2G), although transient knockdown of ST3GAL1 (which catalyzes the transfer of α2,3-SA) via siRNAs had similar cluster-promoting effects as siST6GAL1 (FIG. 11E), suggesting a distinct regulatory mechanism and chemo-selective pressure on ST6GAL1.

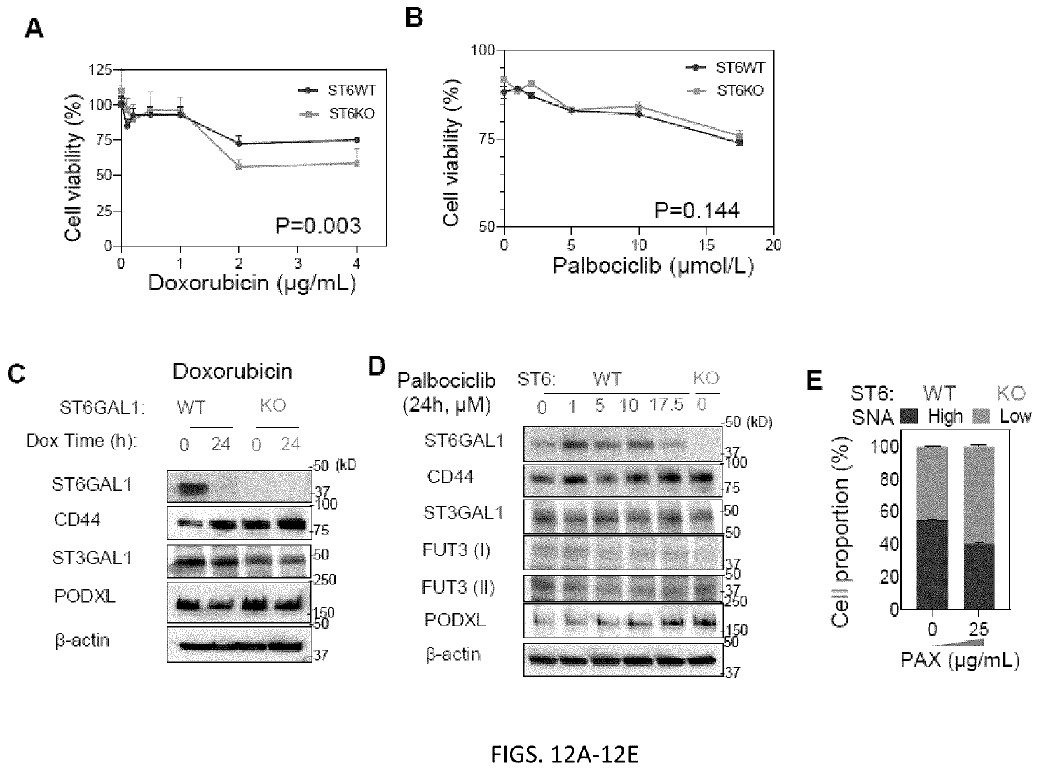

We further determined the effects of ST6GAL1 depletion on cancer cell sensitivity to therapeutic agents, including the chemotherapeutics PAX and doxorubicin as well as the CDK inhibitor palbociclib. Compared with ST6WT cells, ST6KO in MDA-MB-231 tumor cells enabled a specific resistance to PAX treatments (25-200 μg/mL; FIGS. 2G-2I), but with a similar sensitivity to palbociclib and a moderately increased sensitivity to doxorubicin (FIGS. 12A-12D). When SNA-high (WT) and low (ST6KO) cells were mixed at a 1:1 ratio prior to treatment, increased dominance of the α2,6-SA-low (ST6KO) population of MDA-MB-231 cells emerged 24 hours after PAX treatment (25 μg/mL; FIG. 12E), similar to the profiles of CTC-092 cells in response to PAX treatment (FIG. 2D).

### Loss of ST6GAL1 Induces Quiescence in CTC Clusters for Chemoevasion

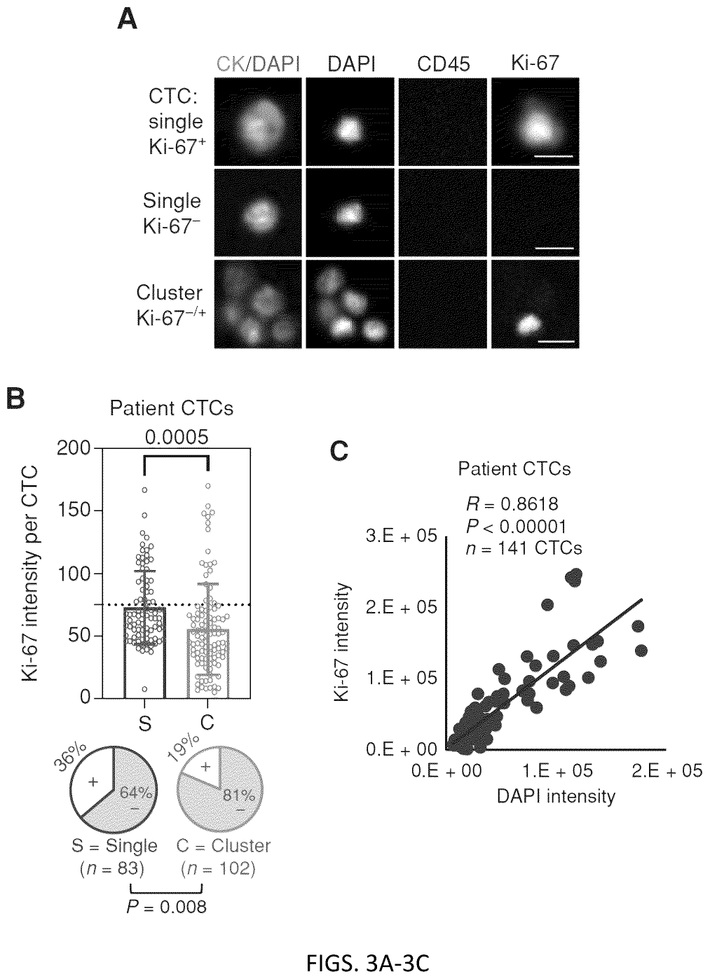

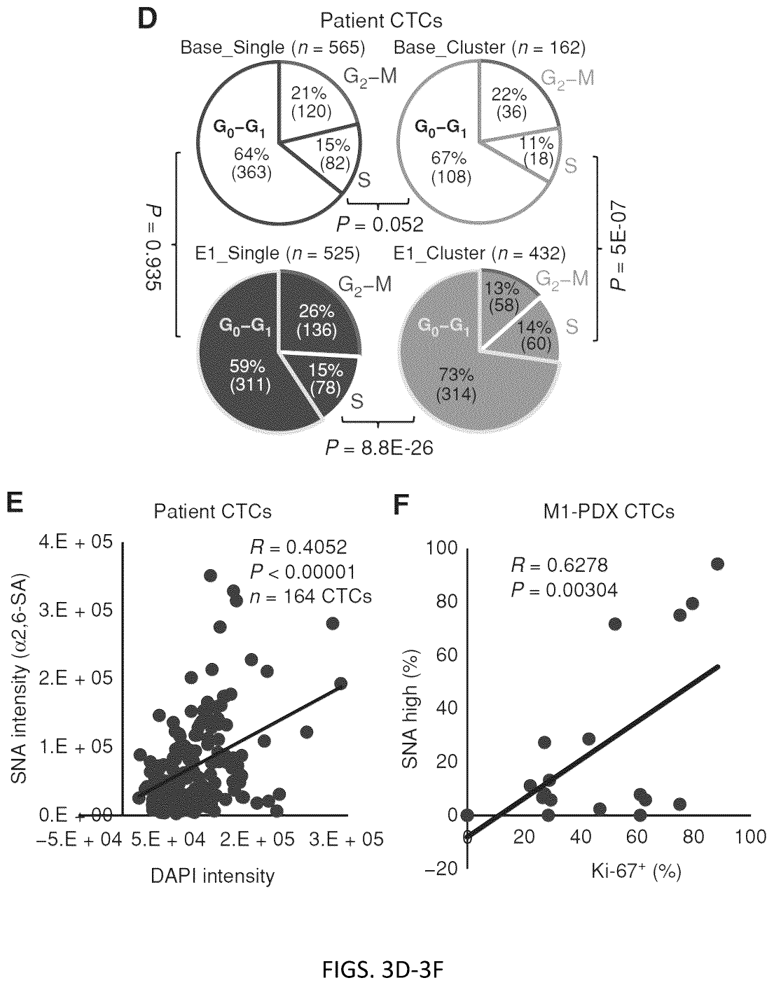

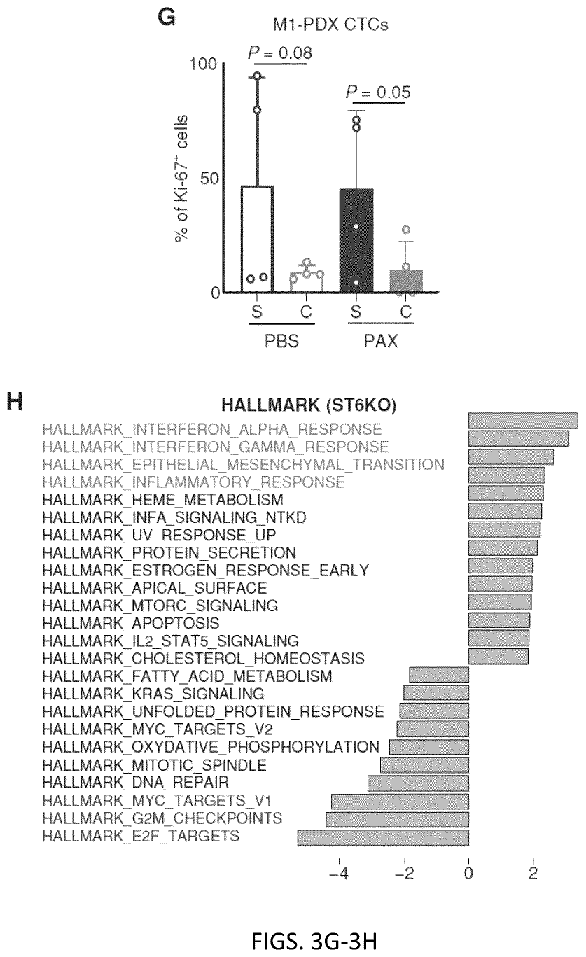

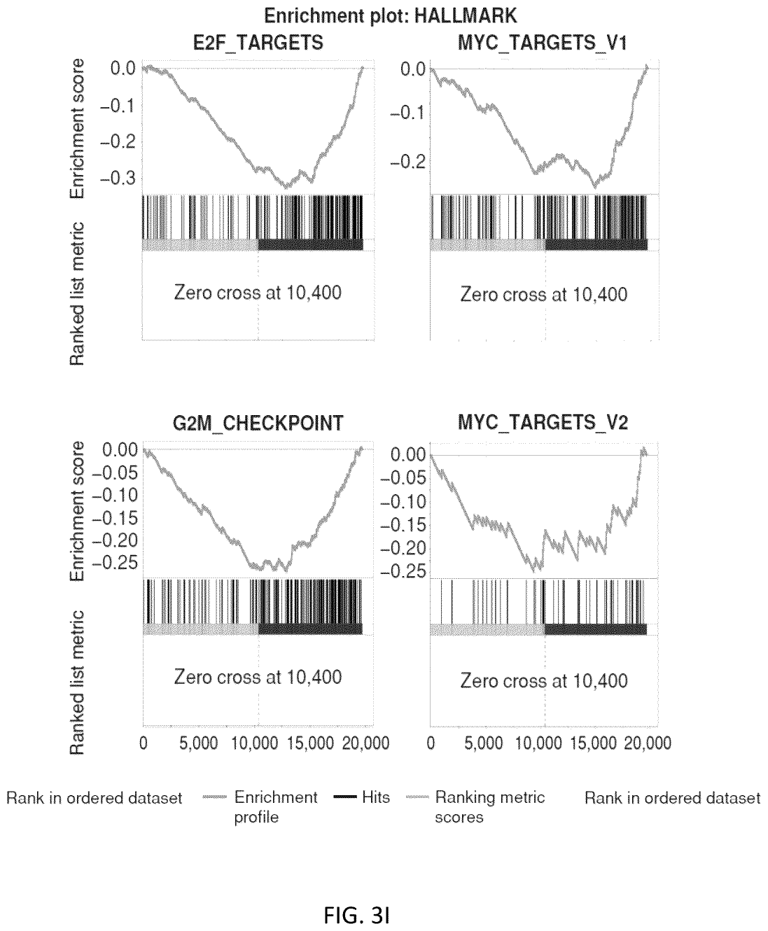

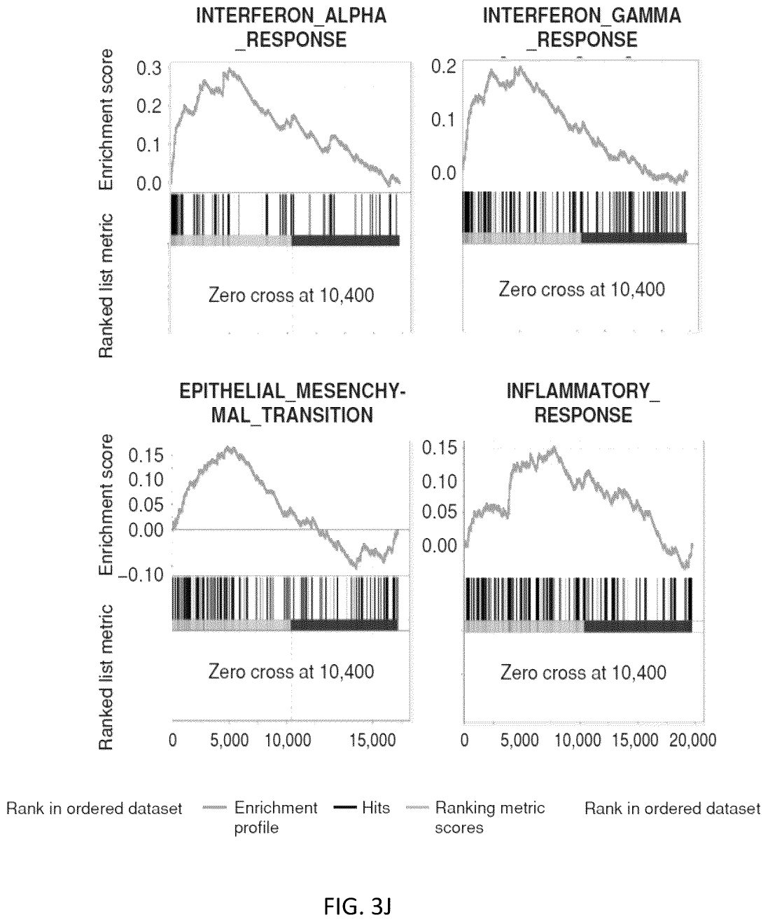

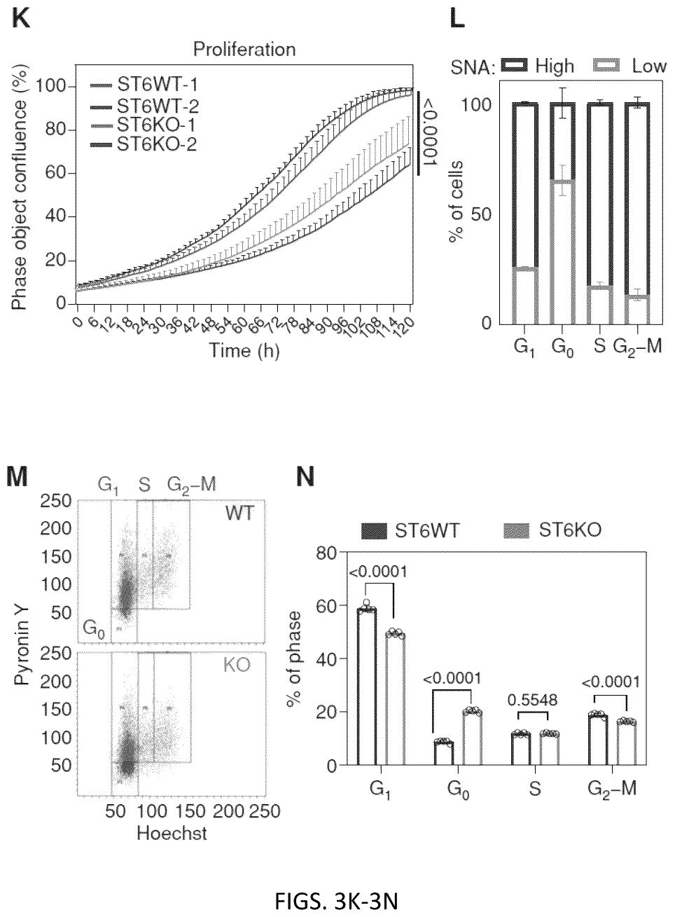

Next, we investigated if chemoevasion of the CTC clusters is associated with distinct proliferative status, as previous studies demonstrated that ST6GAL1 and α2,6-SA are associated with cellular proliferation in various cancers (27-29). We first added customized Ki-67 staining to the patient CTC panel analyses via CellSearch. Through binary assessment and intensity comparison, decreased Ki-67 positivity was observed in clustered CTCs in comparison with single CTCs from patients with breast cancer (FIGS. 3A and 3B, P=0.0005 and 0.008). The heterogeneous levels of Ki-67 signal intensity per cell in singles and clusters positively correlated with the DAPI signal intensity (DNA content) in each CTC (FIG. 3C). The cell-cycle analyses based on DAPI intensity revealed that the population of G1-G0 phase increased in clusters after chemotherapy (73%) more than that of single CTCs (59%), whereas single and clusters at baseline prior to therapy were 64% and 67%, respectively (FIG. 3D). Moreover, the intensity of DNA content (DAPI intensity) is in positive correlation with SNA signals (α2,6-SA levels) per human CTC (FIG. 3E). We then continued to measure the α2,6-SA levels and Ki-67 positivity of CTCs in TNBC PDX models that develop spontaneous lung metastases (4). Flow cytometry analyses revealed that the SNA-high levels (%) of L2T⁺/L2G⁺ CTCs from the PDX-M1 tumor-bearing mice also correlated with high Ki-67 positivity (FIG. 3F). Furthermore, in PAX-treated mice, the Ki-67 positivity (%) within CTC clusters was significantly lower than single CTCs (FIG. 3G), indicating an association of CTC clusters with low α2,6-SA levels and relative quiescence under the pressure of PAX treatment.

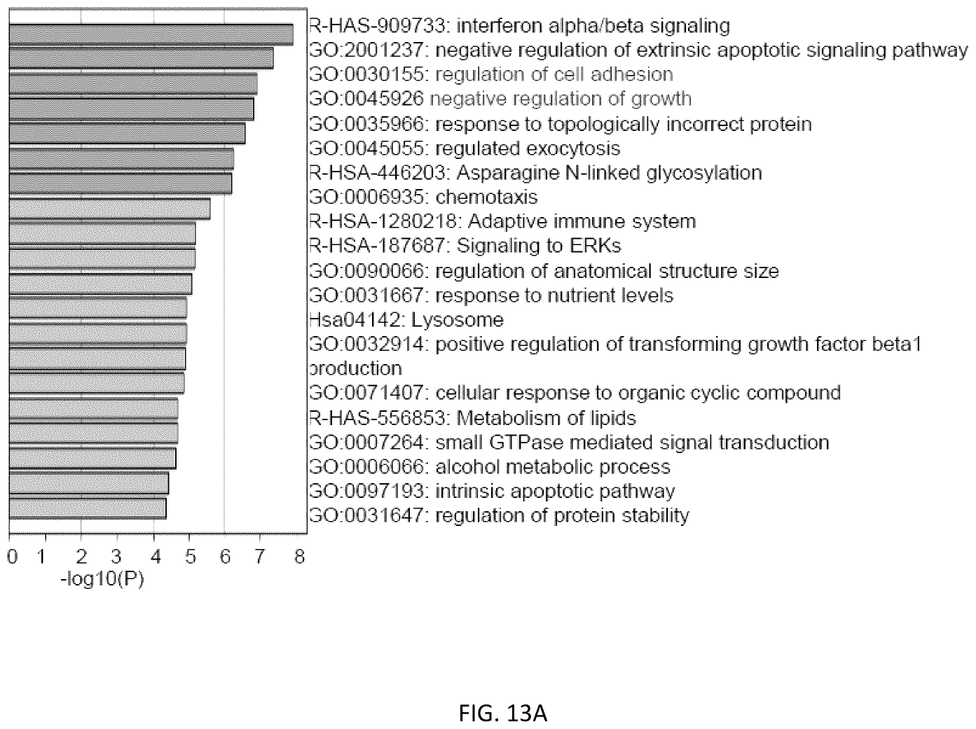

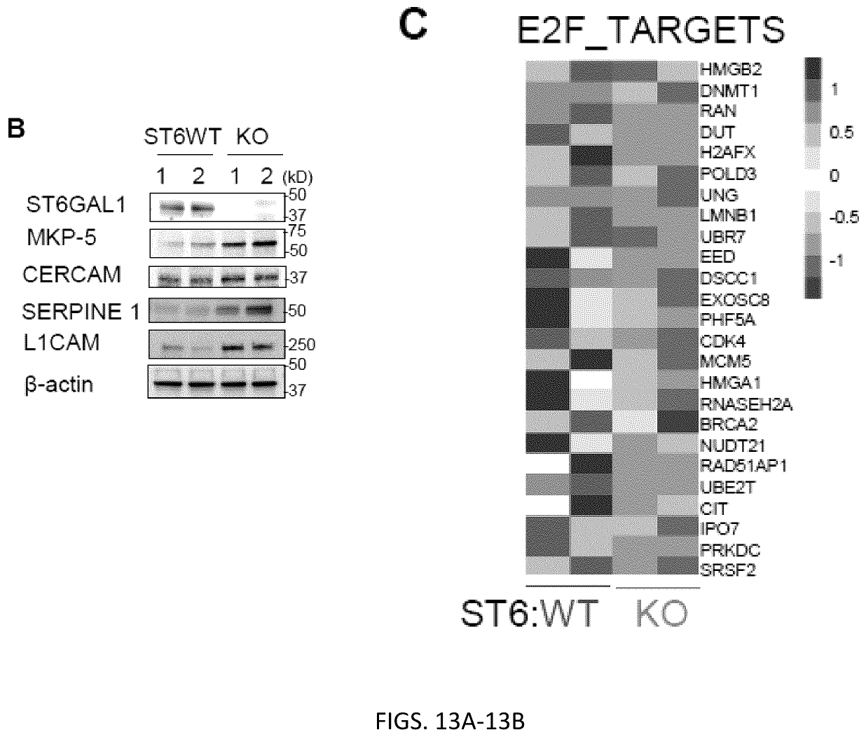

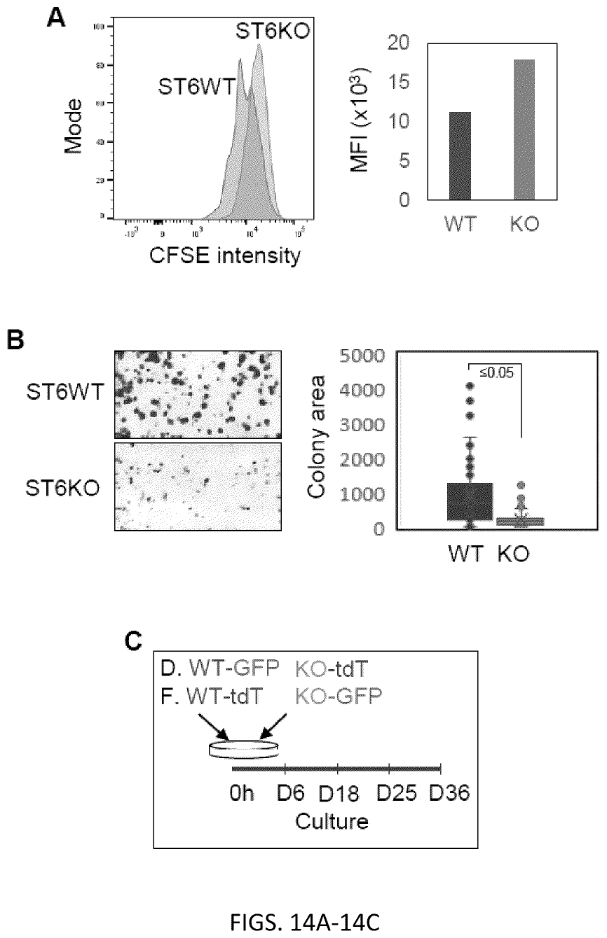

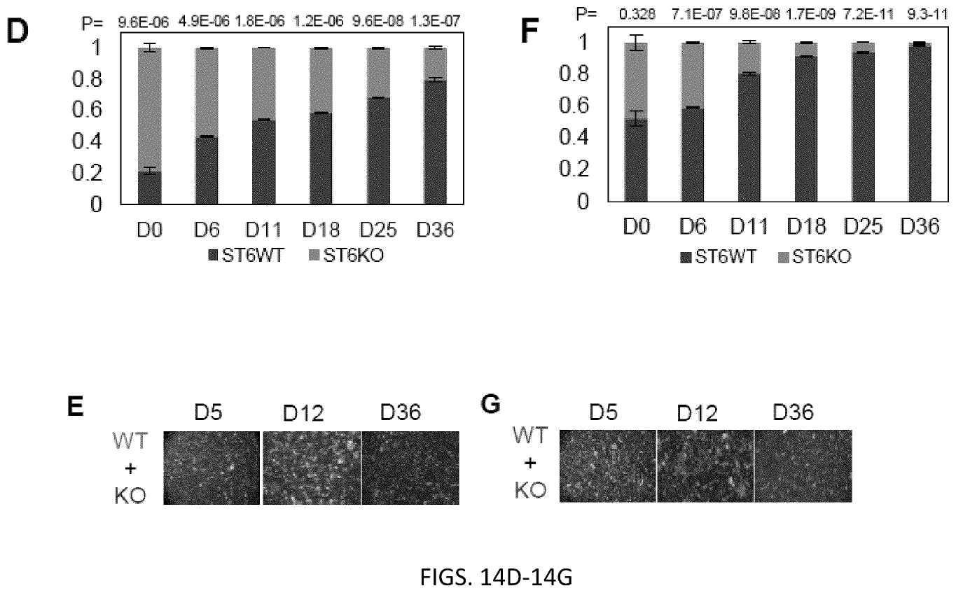

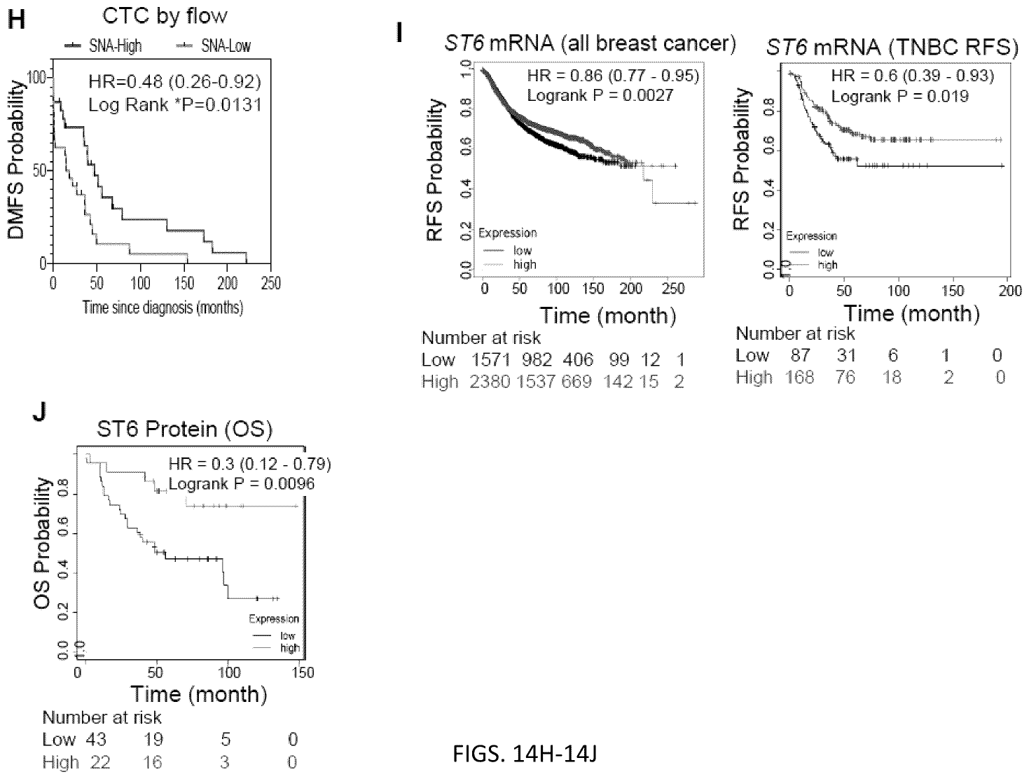

To further elucidate the transcriptome programs related to low α2,6-SA levels (ST6GAL1 depletion) and quiescence, we conducted RNA-sequencing (RNA-seq) analysis of the WT and ST6KO bulk tumor cells. Hallmark pathway analysis and gene set enrichment analysis identified differential regulatory pathways in ST6KO tumor cells, including suppressed cell cycling, such as E2F targets, G₂-M check-points, and MYC targets, as well as upregulated interferon responses, cell adhesion, epithelial-to-mesenchymal transition, and inflammatory responses (FIGS. 3H-3J; FIGS. 13A-13C). As the RB-E2F signaling pathway controls proliferation and quiescence in cancer cells (30-32), we analyzed cell growth and cell-cycle phases of the tumor cells using multiple approaches. ST6KO cells showed a delayed growth rate for confluency compared with the WT cells (FIG. 3K). Using Hoechst and pyronin Y-based DNA and RNA (respectively) double staining, we found that α2,6-SA-low (SNA-low) WT cells dominated the quiescent pyronin Y⁻(G0) phase but contributed to only a small subset of active cycling phases, G₁, S, and G₂-M (FIG. 3L). Furthermore, compared with ST6WT cells, ST6KO increased the proportion of tumor cells in quiescent/G₀ phase and decreased in active cell cycles such as G₁ and G₂-M (FIGS. 3M and 3N), with a consistent pattern of delayed and halted cell division with relatively undiluted dye of carboxy-fluorescein succinimidyl ester (CFSE) in the KO cells (FIG. 14A). Furthermore, ST6KO cells formed significantly smaller colonies than ST6WT cells (FIG. 14B). When ST6WT and KO cells were mixed in two distinct colors for competitive proliferation assays in culture (FIG. 14C), the counts of ST6KO cells were overtaken by ST6WT cells over time (FIGS. 14D-14G). However, the dominance was converted to ST6KO cells under PAX treatment at an even lower dose of 25 μg/mL (FIG. 12E). These results suggest that lack of ST6GAL1 induces cellular quiescence at G₀ and provides advantages to chemoevasion.

Because chemoresistance is often associated with local relapse and distant metastasis, we continued to investigate the clinical relevance of α2,6-SA and ST6GAL1 levels in CTCs or tumor cells with breast cancer outcomes. Clinically, we observed an unfavorable distant metastasis-free survival (since the diagnosis) of the patients with breast cancer and detectable SNA-low CTCs (FIG. 14H). Furthermore, using the online Kaplan-Meier plotter analysis (33), we found that high expression of ST6GAL1 mRNAs is associated with a favorable relapse-free survival in TNBC (n=265, P=0.019) and higher protein levels of ST6GAL1 in breast cancer predict a dramatically better OS than those with lower protein levels (n=65, P=0.0096; FIGS. 141 and 14J). These data prompted us to investigate the alteration patterns and molecular mechanisms associated with the impact of ST6GAL1 in metastasis.

### Dynamic Changes of α2,6-SA and ST6GAL1 Levels in CTCs and Disseminated Tumor Cells During Seeding

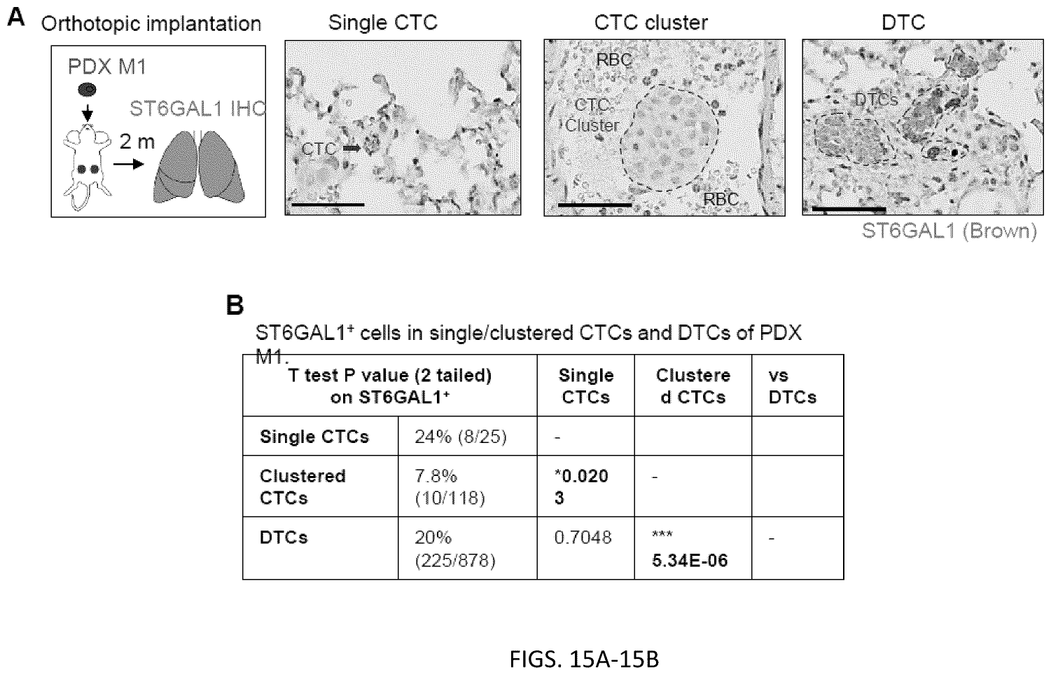

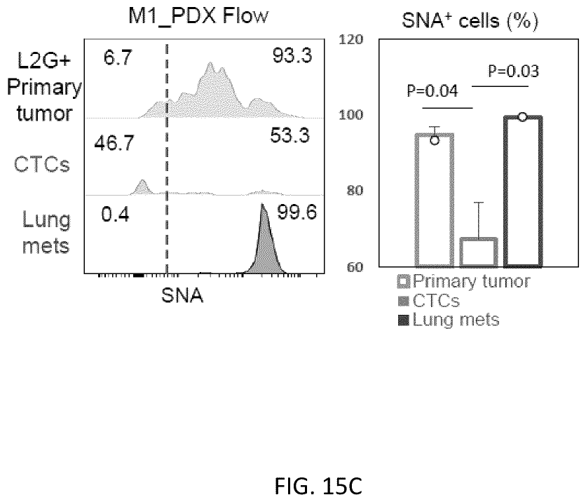

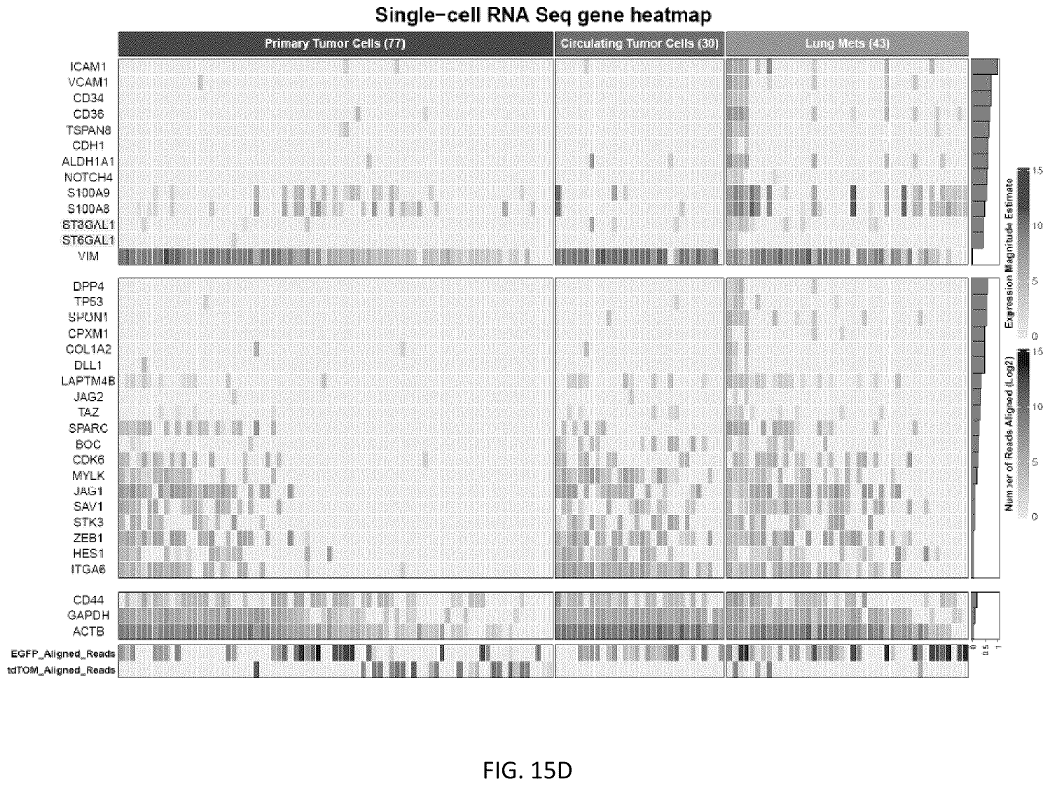

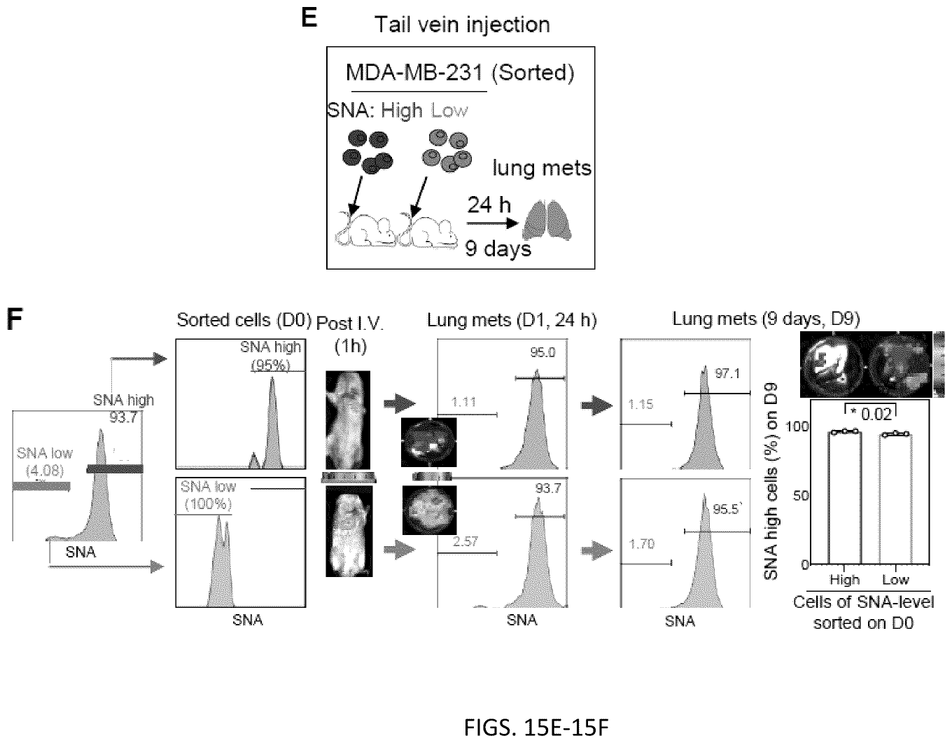

To determine the patterns of α2,6 sialylation in CTCs and disseminated tumor cells (DTC) during metastasis, we compared their ST6GAL1 expression profiles and α2,6-SA levels using IHC staining and flow cytometry, respectively. In PDX-M1 orthotopic tumor models, the lung tissue sections with spontaneous metastases were stained with anti-human ST6GAL1, showing a significantly higher ST6GAL1 positivity (%) in single CTCs and DTCs than CTC clusters (FIGS. 15A and 15B). Analyses of SNA binding to eGFP⁺ (L2G⁺) or tdTomato⁺ (L2T⁺) primary tumor cells, CTCs, and lung metastases from TNBC PDX-M1 also revealed lower α2,6-SA levels in CTCs than both primary tumor cells and lung metastases, which regained higher levels of α2,6 sialylation after seeding (FIG. 15C). Single-cell RNA-seq of primary tumor cells, CTCs, and lung metastasis from our PDXs and MDA-MB-231 tumors (4) also revealed low expression of ST6GAL1 in CTCs (FIG. 15D), suggesting possible plasticity and dynamic α2,6 sialylation patterns in CTCs during metastatic seeding. Then, we sorted SNA-high and SNA-low cells of MDA-MB-231 cells for tail-vein injection into mice, and the lungs were dissected and dissociated for flow cytometry analyses of L2G⁺/L2T⁺ tumor cells. Surprisingly, the SNA-low tumor cells (sorted as 100% SNA-low) shifted to nearly all SNA-high (>90% high) DTCs in the lungs, as detected at 24 hours and 9 days after intravenous injection (FIGS. 15E and 15F), suggesting a transient loss of α2,6-SA in CTCs and a dynamic gaining of sialylation in DTCs after seeding. This finding led us to determine the regulatory roles of ST6GAL1 in metastasis in TNBC.

### Depletion of ST6GAL1 Promotes Metastatic Seeding of TNBC

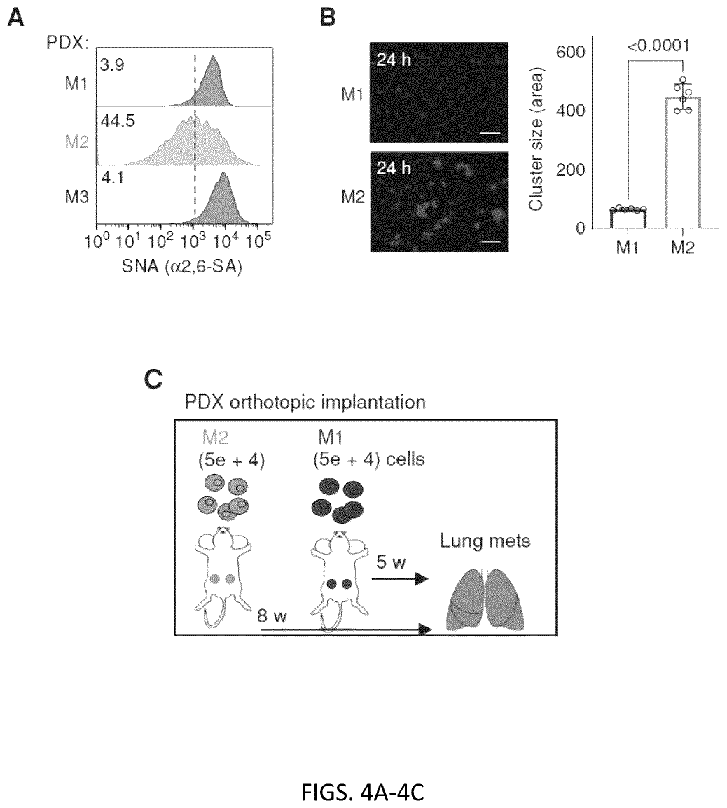

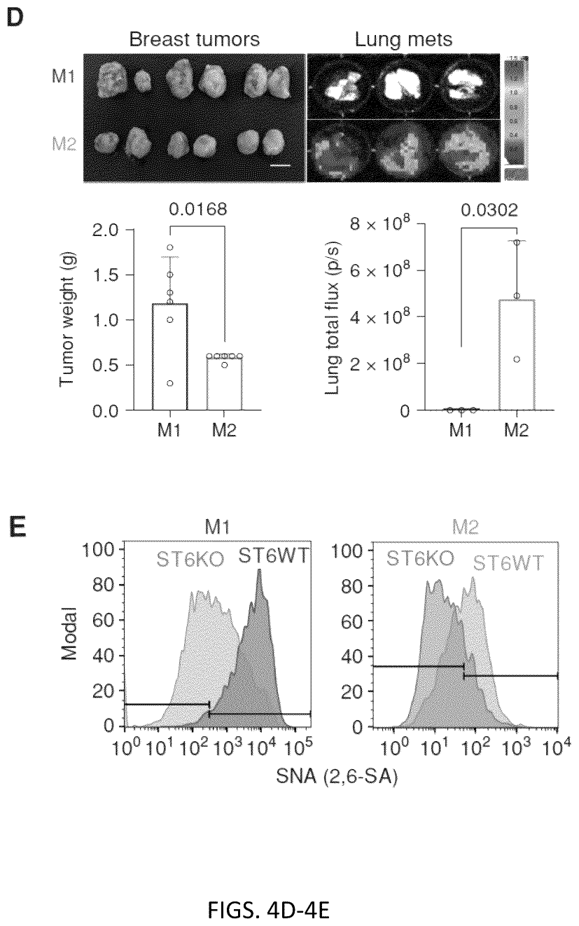

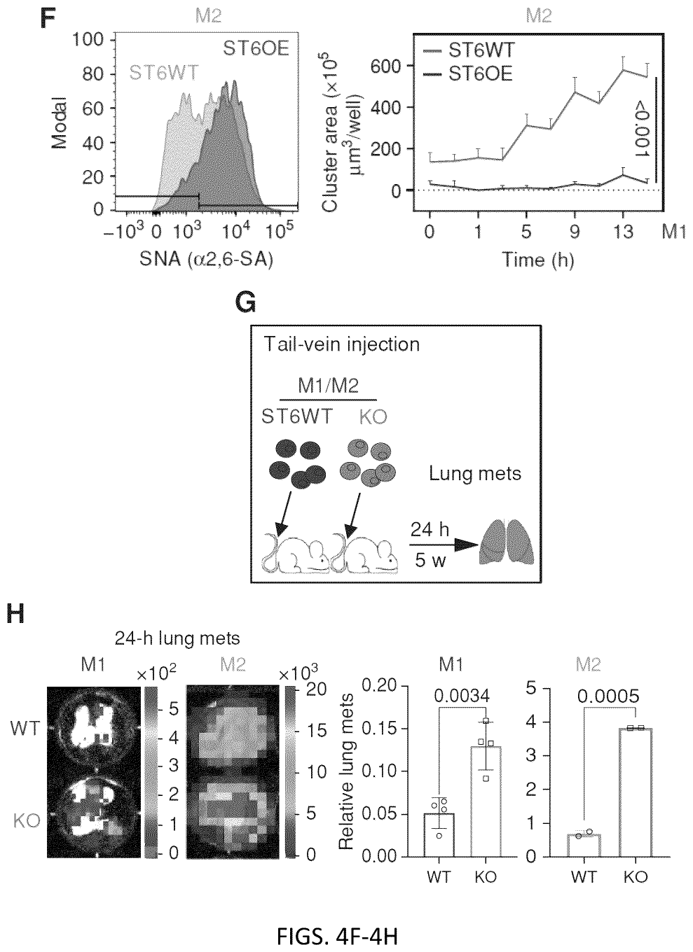

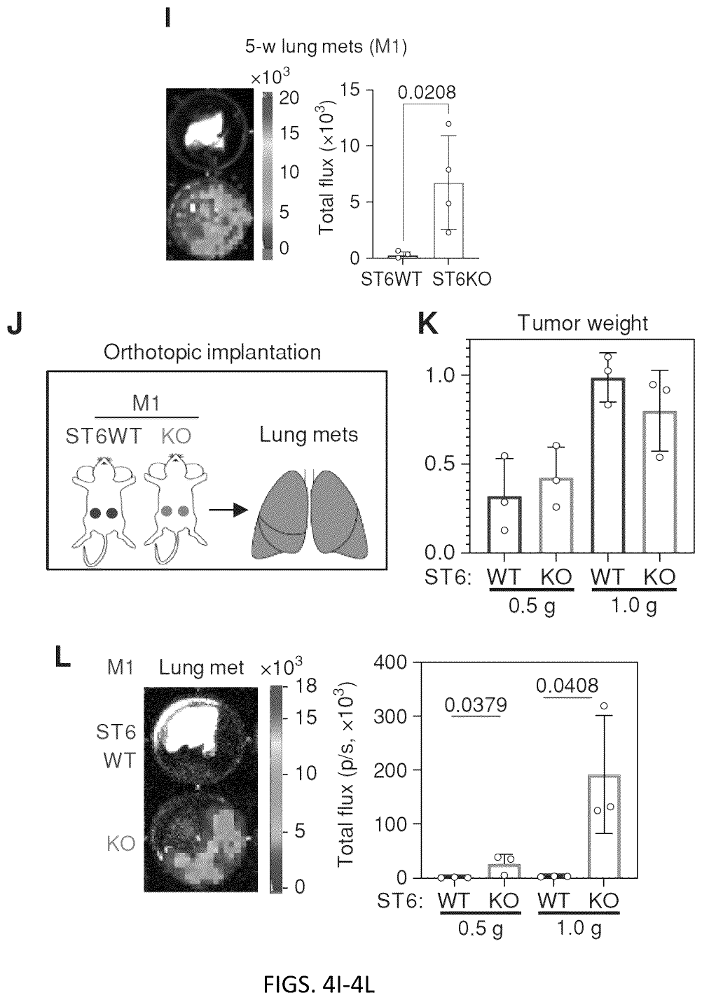

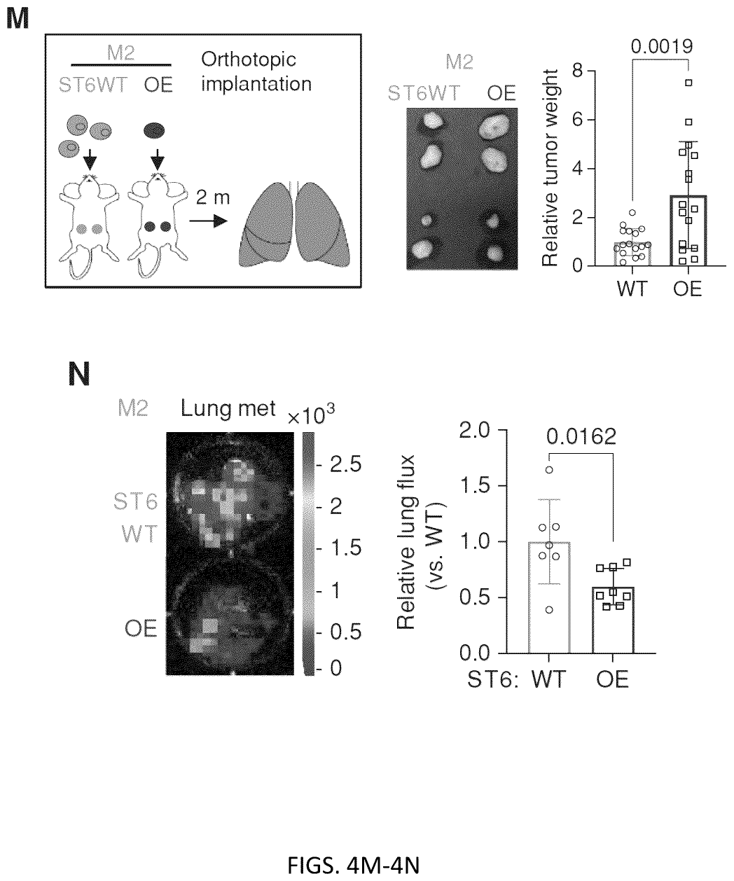

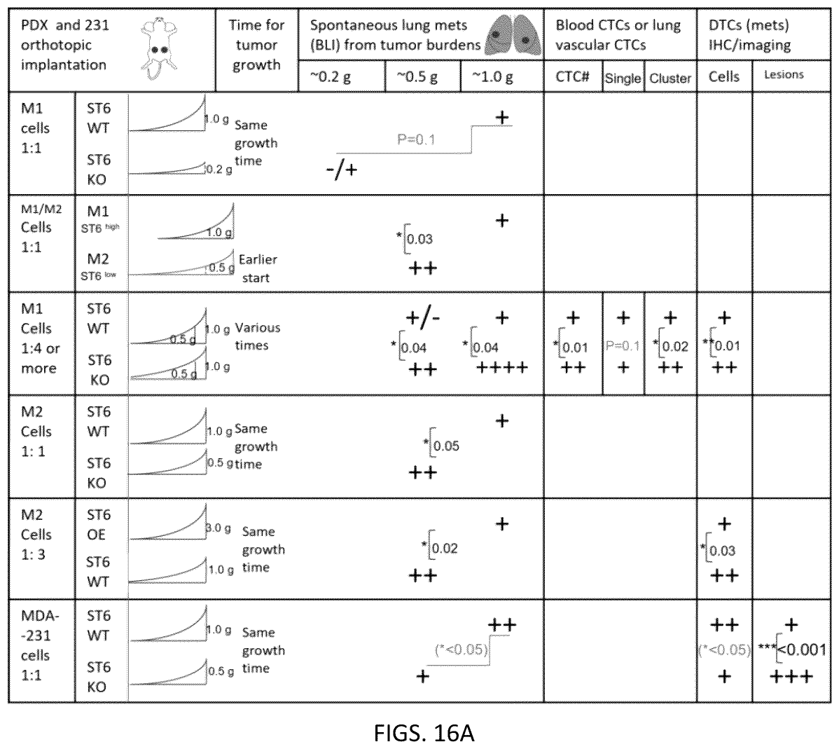

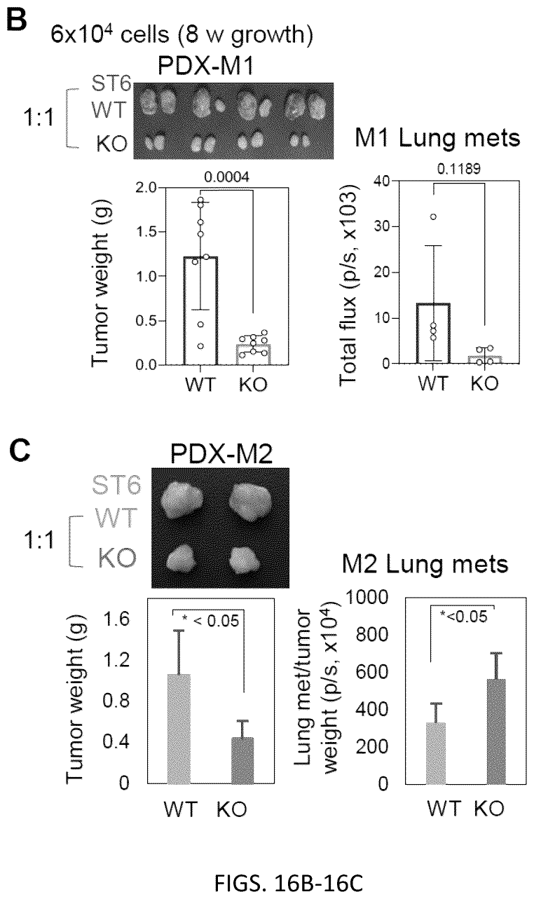

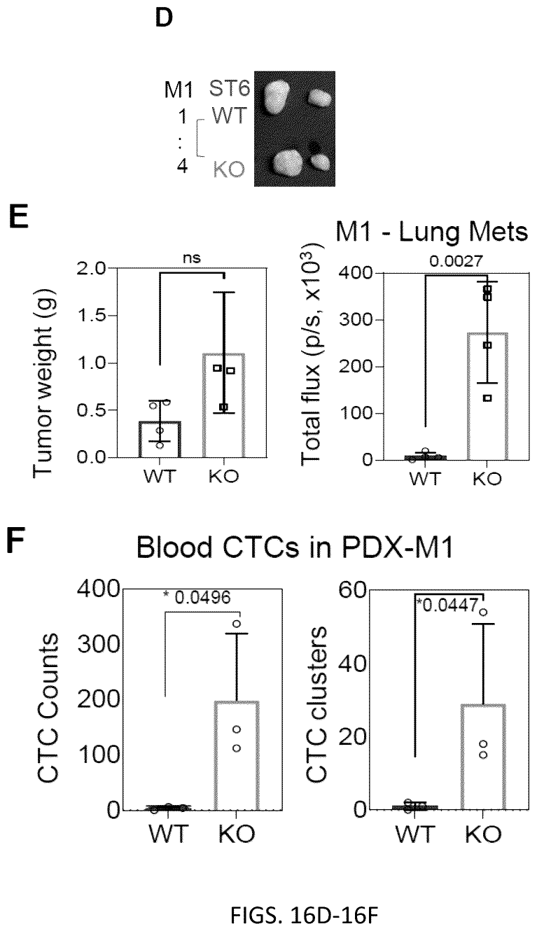

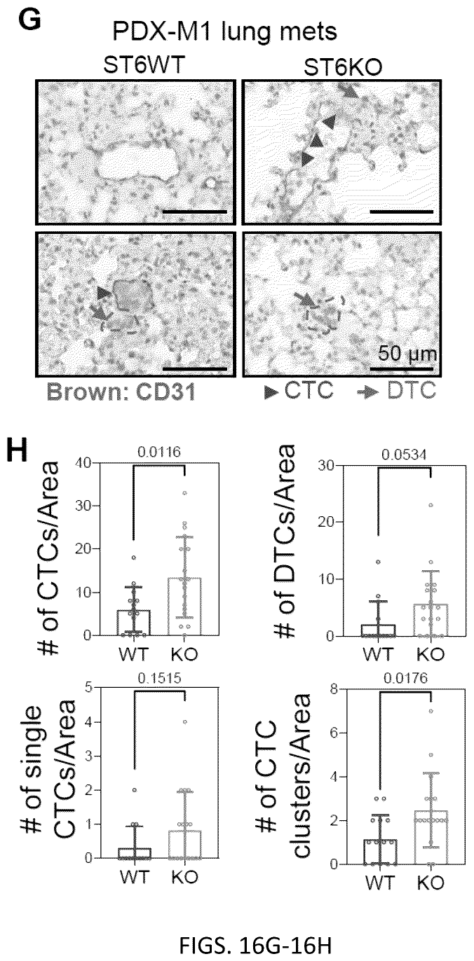

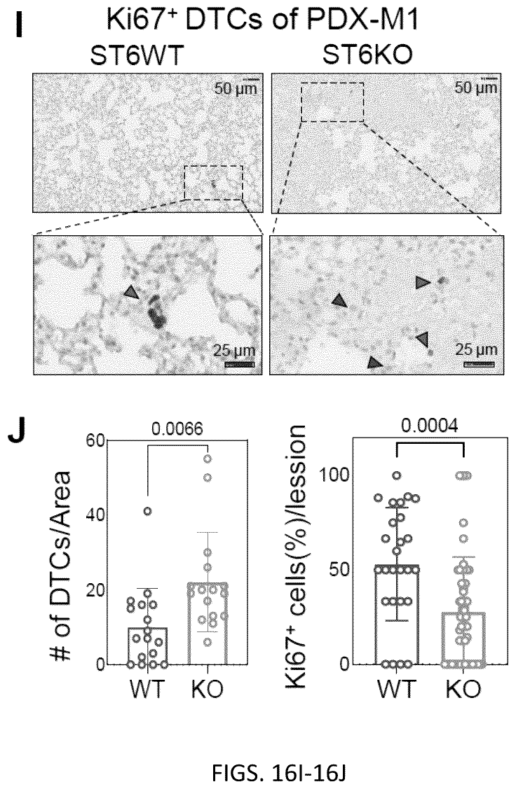

Based on the glycan profiles of multiple TNBC PDX tumors (8), we identified SNA-high M1 and M3 PDXs (>95% SNA+) and SNA-low M2 PDXs (˜45% SNA+; FIG. 4A), which were labeled by the dual optical reporters L2G or L2T (8) for bioluminescence and fluorescence imaging (4). When placed on collagen-coated plates ex vivo for aggregation-based clustering (4), dissociated SNA-low M2 tumor cells formed 5-fold larger clusters than the SNA-high M1 tumor cells (FIG. 4B). To compensate for the potential disadvantages of smaller tumors for metastasis, we designed a series of tumor implantation experiments for slow-growing ST6-deficient tumor cells to reach tumor volumes relatively close to ST6WT or SNA-high tumors for assessment of metastatic seeding and lung colonization. To determine the regulatory roles of ST6GAL1, the entire in vivo metastatic analysis of both ST6WT and ST6KO cells is shown in FIG. 16A.

We first compared the lung metastases developed by SNA-high M1 and SNA-low M2 PDX models (FIG. 4C). Following orthotopic implantations of equal numbers of tumor cells into mouse mammary fat pads but at different timing, we observed a slower growth rate of the SNA-low M2 PDXs, which within an even extended time window grew about half the weight of the SNA-high M1 tumors; however, these smaller M2 tumors developed spontaneous lung metastases with bioluminescence signals in 108-fold higher intensity than that of M1 PDXs (FIG. 4D), suggesting that low α2,6-SA levels are associated with an elevated metastatic potential in PDX models. To determine if genetic modulations of ST6GAL1 and α2,6-SA levels impact lung metastasis, we first generated ST6KO with depleted α2,6-SA levels in M1 and M2 PDXs (FIG. 4E) but also overexpressed ST6GAL1 (ST60E) in SNA-low M2 PDX cells in which elevated α2,6-SA levels inhibited tumor cell cluster formation (FIG. 4F).

After tail-vein injections of equal numbers of tumor cells, ST6KO M1 and ST6KO M2 cells seeded 2.5-fold and 5-fold experimental metastases to the lungs within 24 hours compared with their respective ST6WT tumor cell controls (FIGS. 4G and 4H). The elevated seeding of ST6KO tumor cells sustained in colonizing the lungs more effectively than the WT control cells, as detected 5 weeks after injections (FIG. 4I).

After orthotopic implantations of ST6WT and ST6KO tumor cells in identical numbers, we observed a significant delay of tumor initiation and slower growth in ST6KO PDX tumors (both M1 and M2) in comparison with the WT controls (FIGS. 16A-16C). When ST6WT M1 tumors reached ˜1.0 g with significant dissemination, ST6KO M1 tumors at 0.2 g had almost undetectable metastases (FIG. 16B), suggesting a minimal requirement of primary tumor volume for suitable evaluation of metastatic potential. Instead, ST6KO M2 tumors at 0.5 g seeded more spontaneous lung metastases relative to the tumor burden than the WT M2 tumors at the size of ˜1.0 g (FIGS. 16B and 16C).

Furthermore, the ST6WT and ST6KO tumor cells in the PDX-M1 model were implanted at various ratios of cells or within extended time to reach relatively comparable tumor volumes (FIGS. 4J and 4K). With matched tumor weight of 0.5 g or 1.0 g, α2,6-SA-deficient ST6KO M1 tumors developed >20-fold more spontaneous lung metastases than that of ST6WT M1 tumors (FIGS. 4L-4N). A higher number of CTCs and especially CTC clusters were detected in ST6KO M1 tumor-bearing mice than in the WT control via flow cytometry (FIGS. 16D-16F).

### Loss of ST6GAL1 Promotes Transendothelial Migration for Metastatic Seeding

To further assess the extravasation and metastatic seeding of SNA-low CTCs into the lungs, we performed endothelial CD31 staining with the lung tissue sections of PDX tumor-bearing mice to distinguish the vascular CTCs in situ and extravascular DTCs (FIG. 16G). In PDX-M1 tumor models, orthotopically implanted ST6KO tumors disseminated more DTCs to the lungs with a higher number of vascular CTC clusters than the ST6WT tumors but with relatively comparable single CTCs, detected via IHC staining of mouse lung sections (FIGS. 16G-16H). In addition, ST6KO tumors disseminated more DTCs but showed a lower Ki-67 positivity than ST6WT cells (FIGS. 16I-16J).

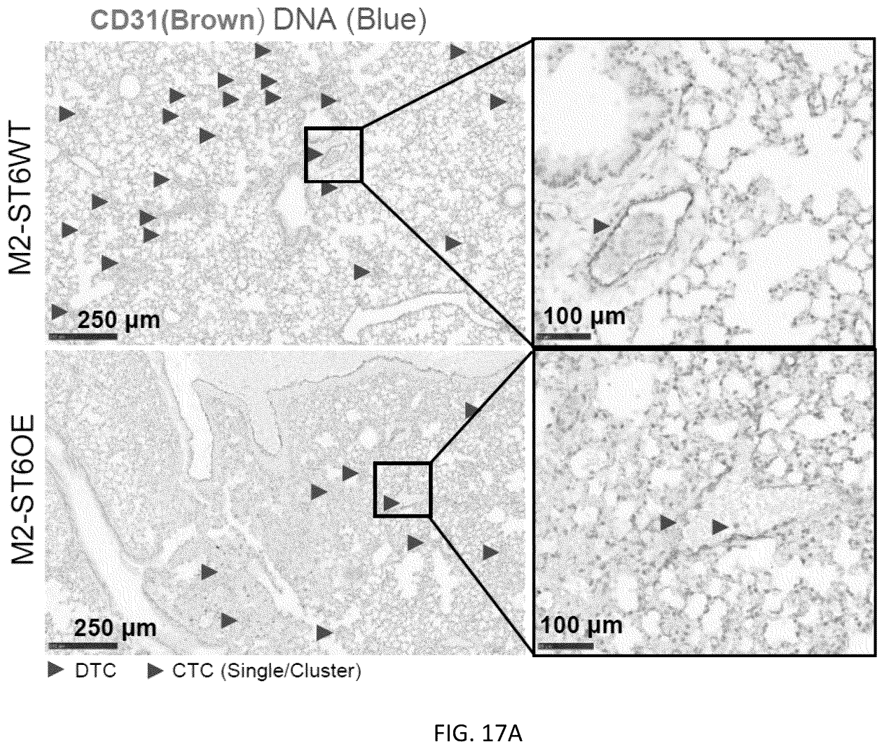

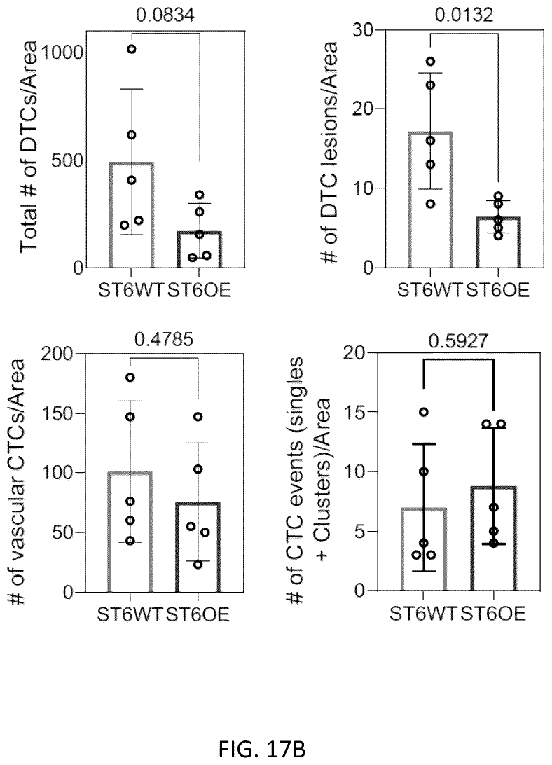

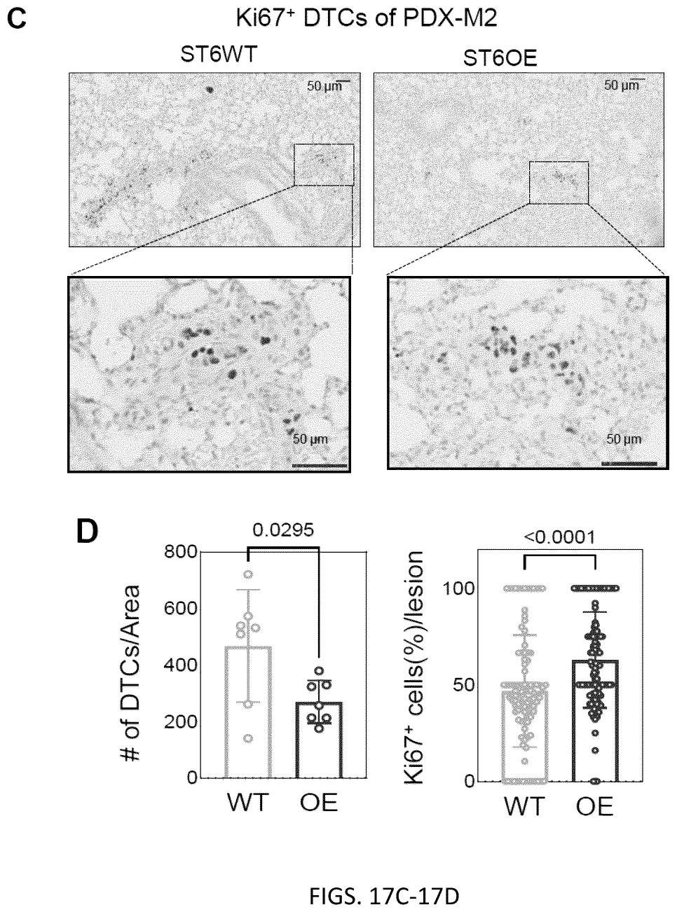

On the other hand, the metastatic potential of PDX-M2 models was assessed with ST6WT and ST60E tumor cells implanted at a 3:1 ratio into separate mice (FIG. 4M). ST60E M2 tumors with an upregulation of α2,6-SA levels grew into >2-fold higher tumor weight than the WT control tumors, but relatively impaired spontaneous lung metastasis, measured via bioluminescence imaging (FIG. 4N). IHC analyses further revealed that ST60E decreased lesions and numbers of DTCs to the lungs, even with a relatively higher rate of Ki-67⁺ DTCs than that of the ST6WT control tumors (FIGS. 17A-17D).

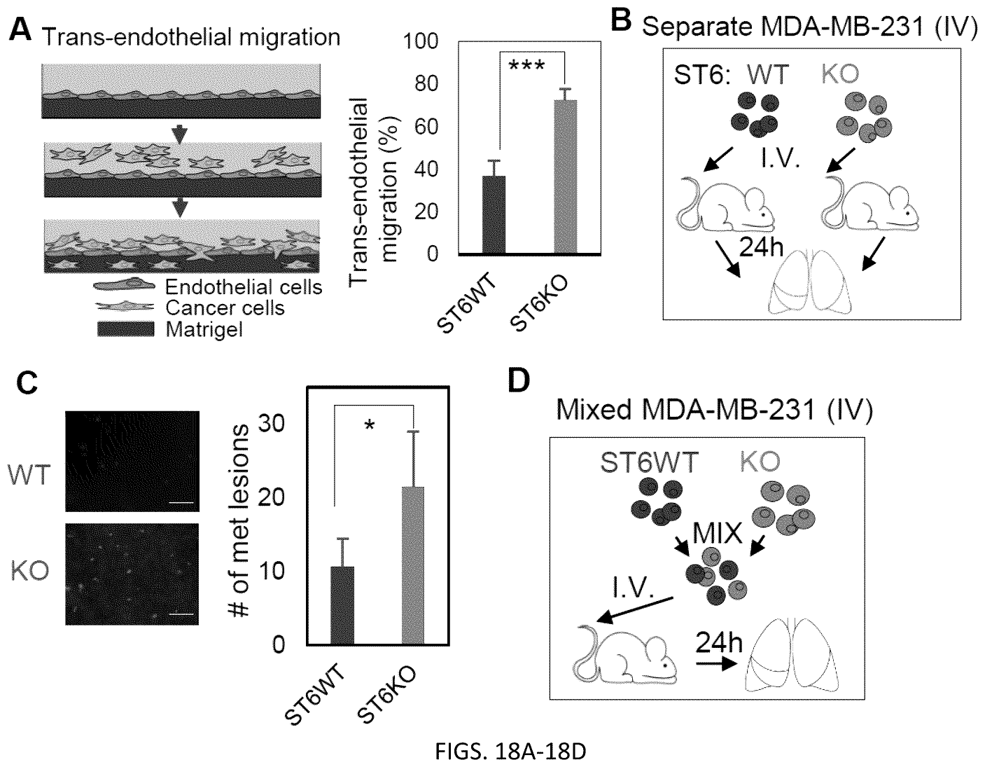

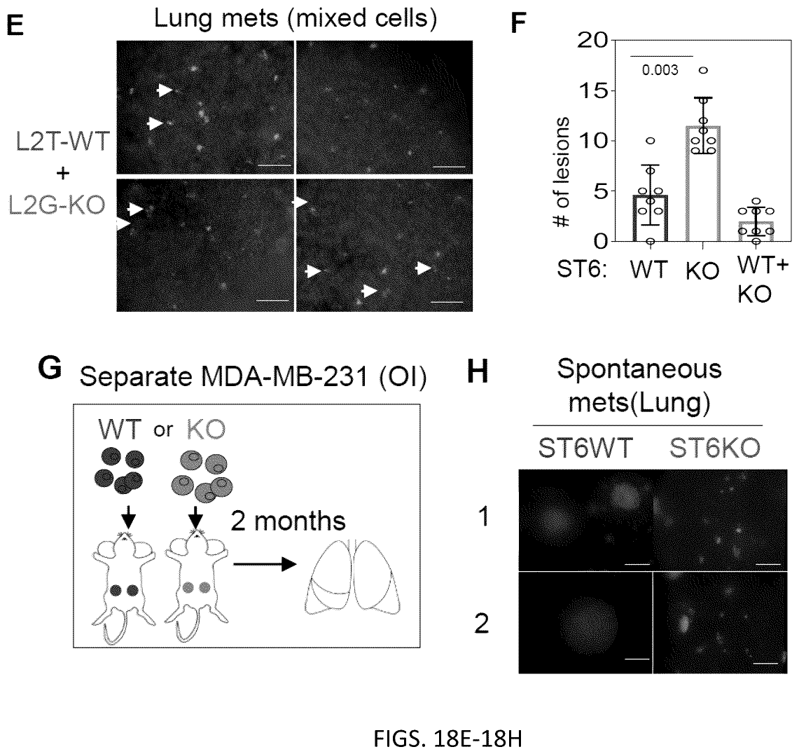

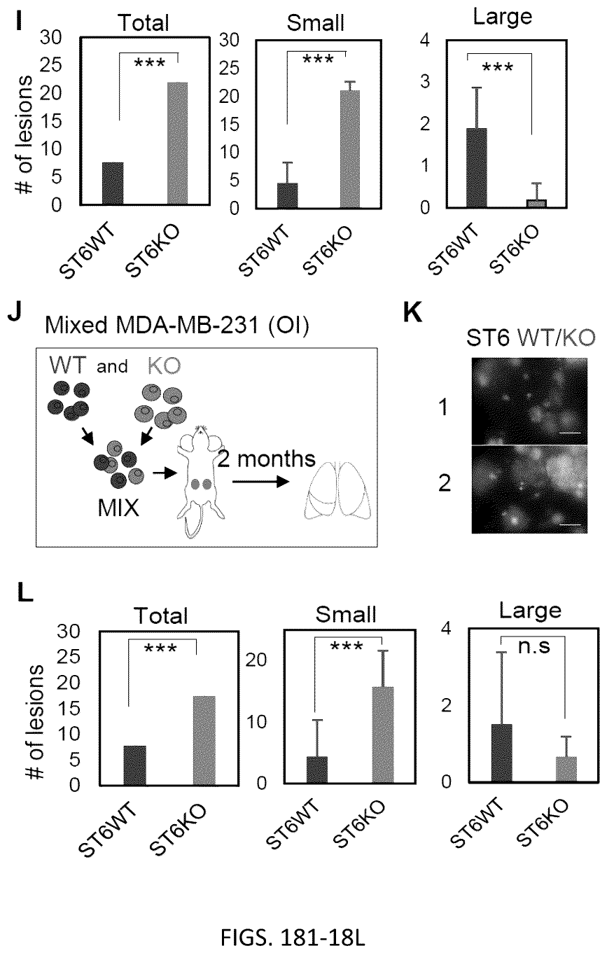

We further evaluated the function of ST6GAL1 in transendothelial migration during metastatic seeding, using our established transendothelial migration assay (34, 35) with tumor cells seeded onto human umbilical vein endothelial cells (HUVEC; refs. 33, 34). Consistently, ST6KO MDA-MB-231 tumor cells with depleted α2,6-SA interacted with and transmigrated through HUVECs at a much higher efficiency than the ST6WT cells (FIG. 18A). Similarly, within 24 hours following tail-vein injections, ST6KO (GFP) cells seeded in the lungs with a dramatically increased number of clusters than ST6WT (tdTomato) cells, in either separately injected or mixed injected (tdTomato-labeled ST6WT with eGFP-labeled ST6KO MDA-MB-231 cells; FIGS. 18B-18F). In addition, following separate orthotopic implantations or mixed implantations into NOD/SCID gamma (NSG) mammary fat pads, ST6KO tumor cells seeded more colonies (total foci) into the lungs but developed into smaller lung colonies compared with ST6WT tumor cells (FIGS. 18G-18L) due to a relatively quiescent phenotype of ST6KO tumor cells (FIGS. 3, 4, 16, and 17).

Together, these results demonstrate double-edge regulatory effects of ST6GAL1, deficiency of which slows down tumor growth but improves CTC clustering and subsequent seeding into the lungs, whereas ST6GAL1 upregulation enhances tumor growth with compromised dissemination and metastasis. However, the dynamics of ST6GAL1 alteration with its transient deficiency in CTCs enables clustering and seeding as rate-limiting steps, whereas restored expression in DTCs empowers secondary regeneration rates in mice.

### Glycoproteomic Analyses Reveal Novel α2,6-Sialylation Substrates of ST6GAL1

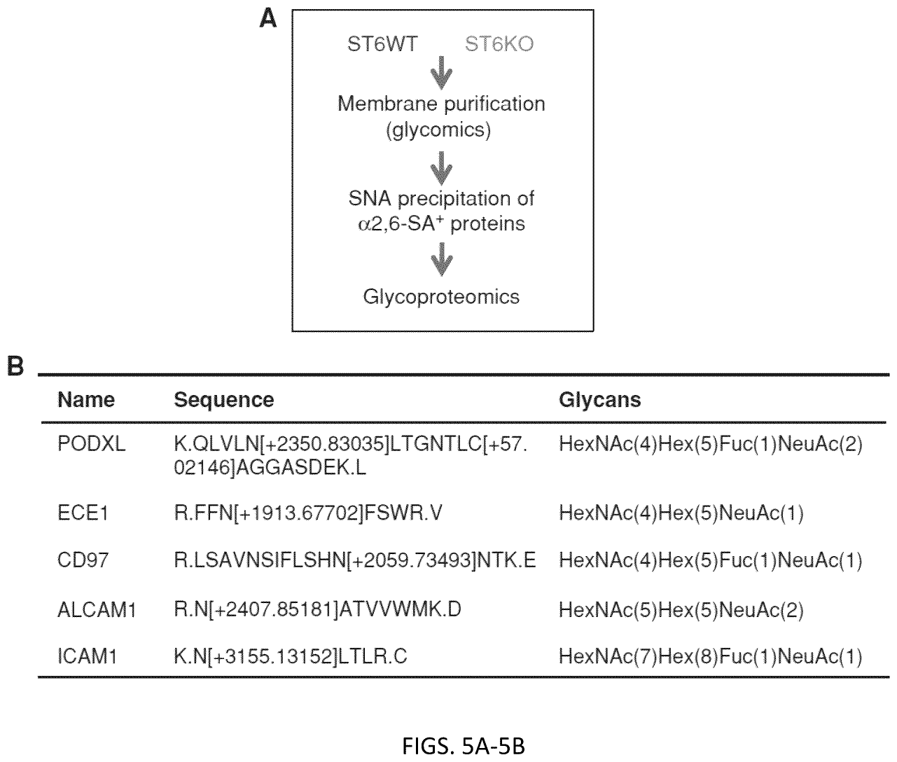

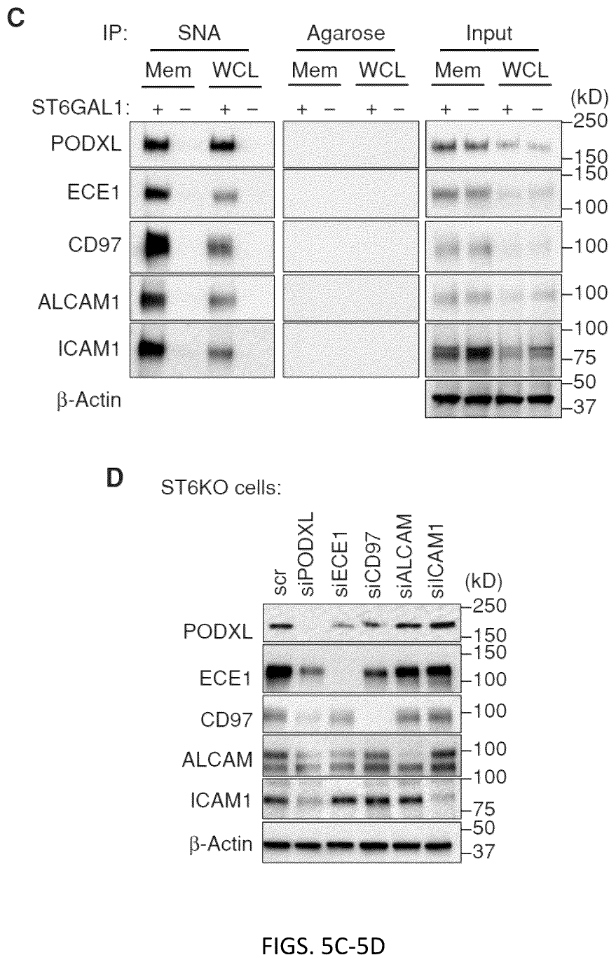

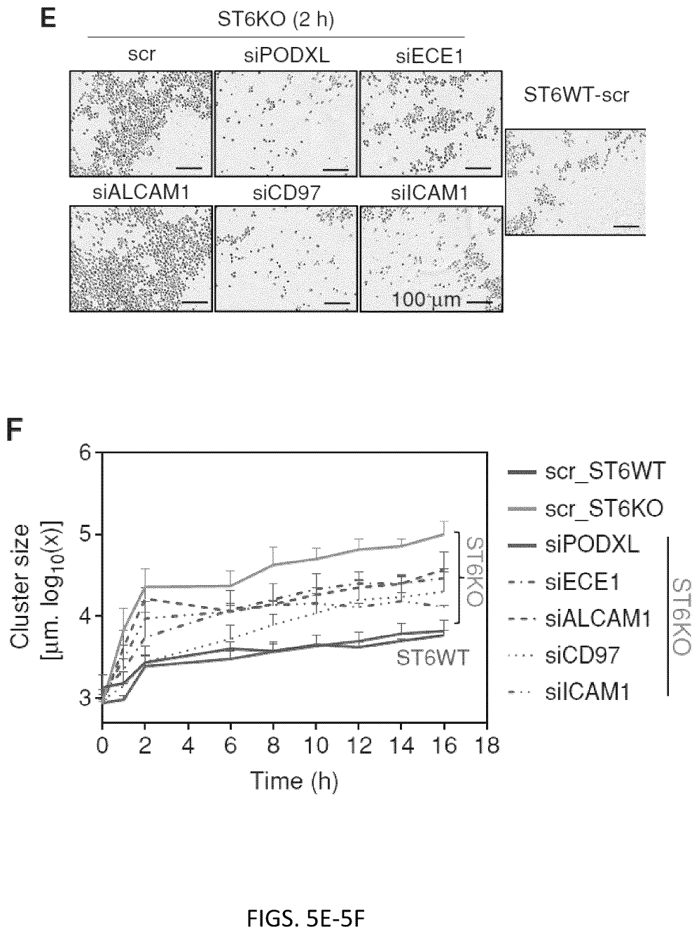

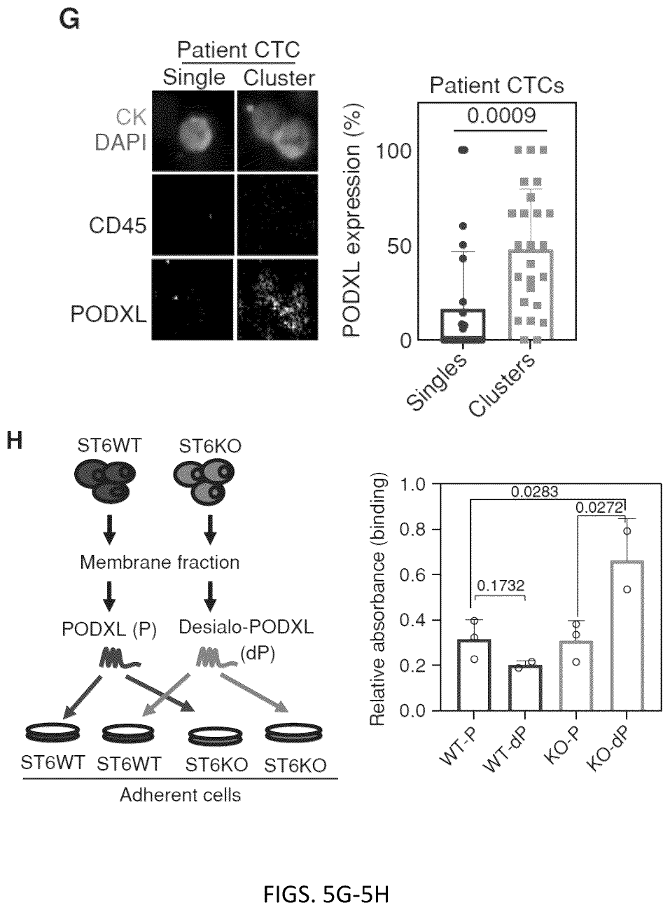

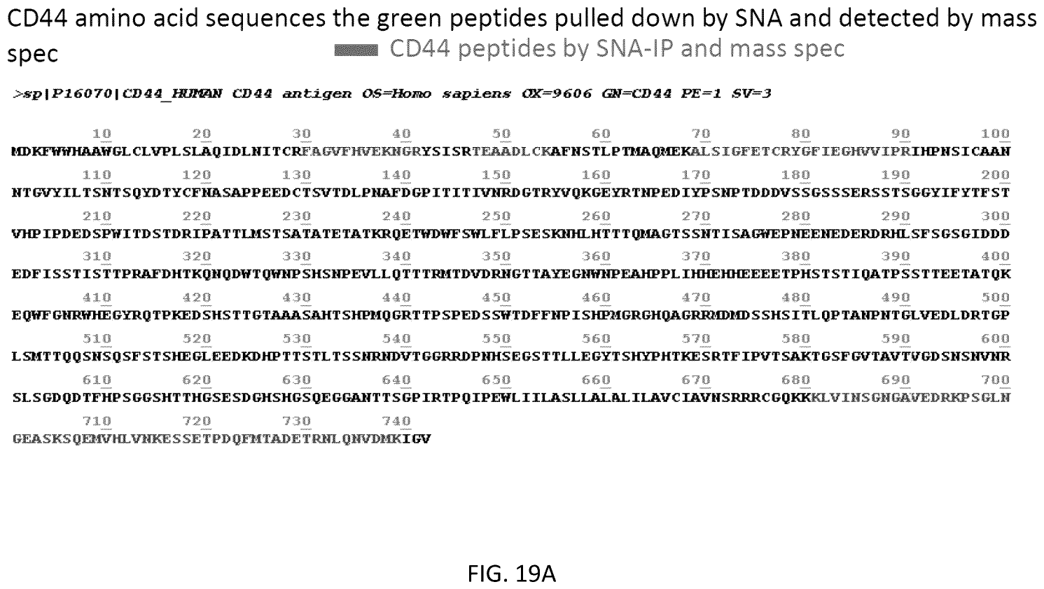

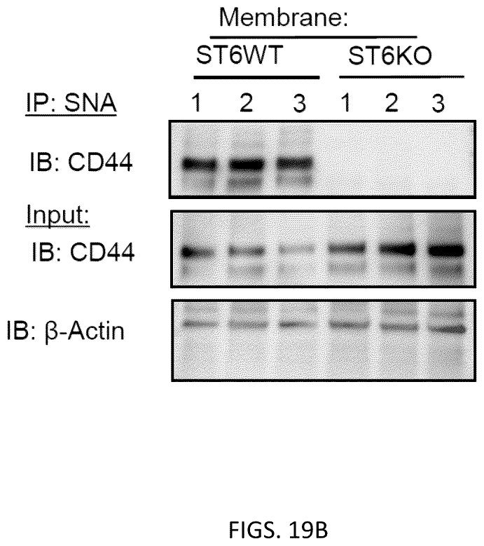

To identify the glycoprotein targets of ST6GAL1 that impact tumor cell cluster formation and metastatic seeding, we performed global glycoproteomic analysis of SNA-precipitated proteins from the membrane fractions or whole-cell lysates of ST6WT and ST6KO cells (MDA-MB-231; FIG. 5A). A total of 217 SNA-bound glycoproteins were specifically identified from the membrane fractions of ST6WT cells in comparison with the negative control of ST6KO cells, including previously known proteins that facilitate CTC cluster formation such as CD44 (4), EGFR (36), ICAM1 (15), and Desmoglein 2 (DSG2; ref. 37; FIG. 19A). Among these glycoprotein targets of ST6GAL1, 68 peptides with characterized N-linked glycans belong to 14 glycoproteins, including known targets of ST6GAL1 (integrin 01) and many newly identified targets such as adhesion molecules PODXL, CD97, ECE1, ALCAM1, and ICAM1, immune regulator HLA-A2, and exosomal proteins CD63, LAMP1, and LAMP2 (FIG. 5B). Immunoblotting of the SNA precipitates further confirmed that PODXL, CD97, ECE1, ALCAM1, ICAM1, and CD44 were enriched in the membrane fractions and specifically pulled down by SNA from WT cells only (FIGS. 5C and 19B).

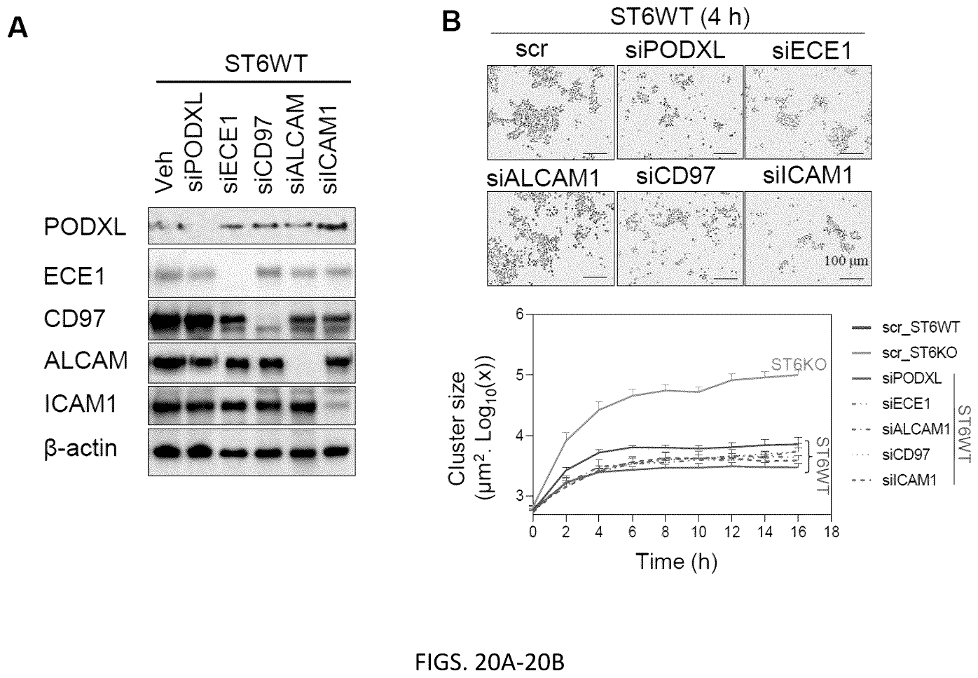

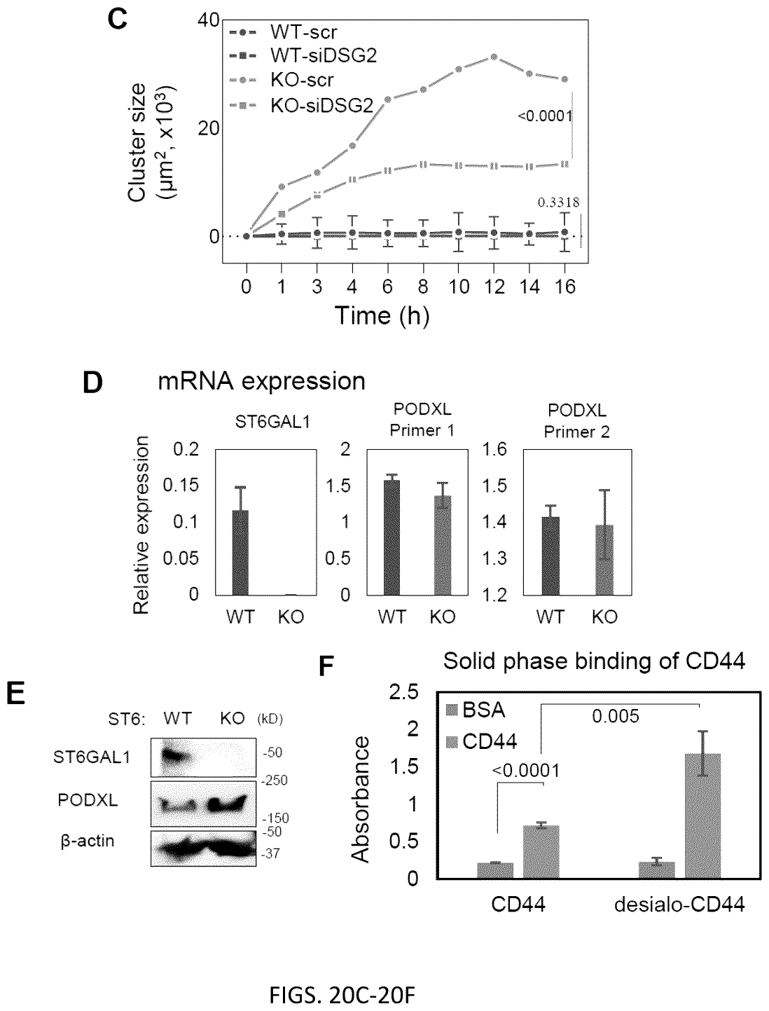

We hypothesized that these adhesion glycoproteins contribute to tumor cell cluster formation and metastatic seeding, and their levels and/or activity are subject to and regulated by ST6GAL1-mediated sialylation. To examine the importance of unknown adhesion glycoproteins in cluster formation, we transfected both ST6WT and KO cells with siRNAs to deplete individual target proteins. Using ICAM1 and DSG2 knockdown as positive controls, downregulation of PODXL, CD97, ECE1, and ALCAM1 also significantly inhibited cluster formation of ST6KO cancer cells, in which PODXL knockdown particularly reversed the clustering level equivalent to the ST6WT cells (FIGS. 5D-5F). Moreover, the knockdown of PODXL and other adhesion molecules in ST6WT cells also slightly suppressed the tumor cell cluster formation except for ALCAM1 and DSG2, which had minimal effects (FIGS. 20A-20C). These data demonstrate that targeting of the downstream adhesion molecules of ST6GAL1 provides possible strategies to partially or completely reverse the tumor cell clustering promoted by the loss of α2,6-SA or ST6GAL1.

We continued to analyze the expression of adhesion molecules in the CTCs of patients with breast cancer using CellSearch and flow cytometry. PODXL expression was significantly enriched in the CTC clusters in comparison with single CTCs isolated from the blood of patients with breast cancer (FIG. 5G). An increased protein expression level of PODXL was also observed in ST6KO cells without an alteration of mRNA expression (FIGS. 20D-20E), implicating the potential posttranslational advantages of desialylated PODXL. Furthermore, we isolated sialylated and desialylated PODXL protein from respective ST6WT and ST6KO cell membrane fractions for cellular binding. The dP-KO binding between desialylated PODXL (dP) and ST6KO cells was stronger than other pairs of bindings, such as dP-WT (dP binding with ST6WT cells) and P-KO (sialylated PODXL binding with ST6KO cells; FIG. 5H). Consistently, after NA treatment, desialylated CD44 increased binding affinity for its homophilic interactions on solid phase (FIG. 20F). This suggests that ST6GAL1-mediated α2,6-sialylation of PODXL may negatively regulate protein stability and/or inhibit their binding activity necessary for tumor cluster formation. The molecular mechanism of ST6GAL1-mediated stability of PODXL remains in our future investigation.

### Targeting PODXL Blocks Lung Metastasis Promoted by ST6GAL1 Deficiency and Chemoevasion

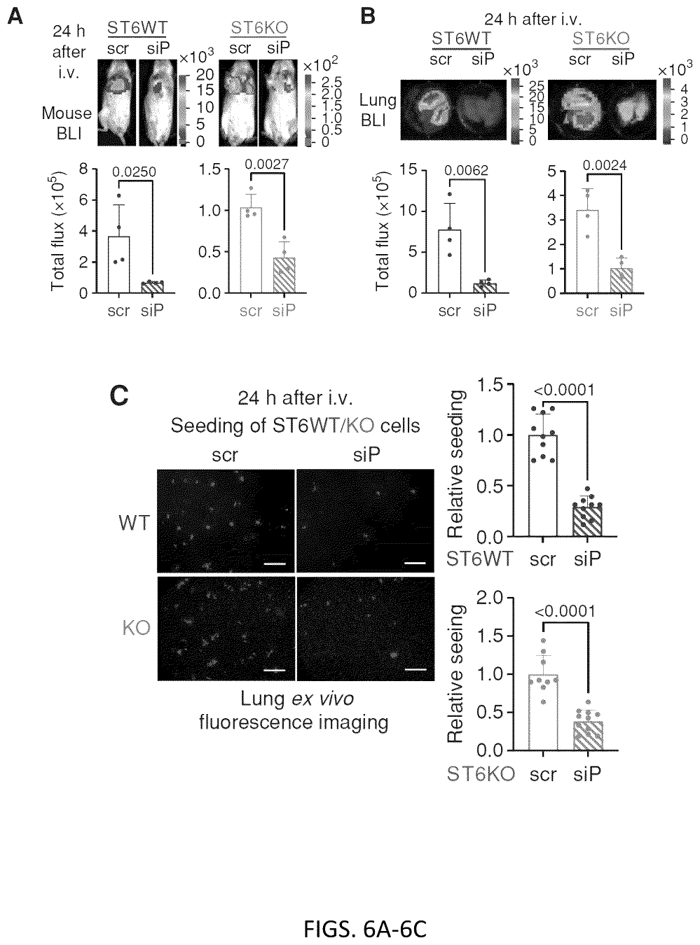

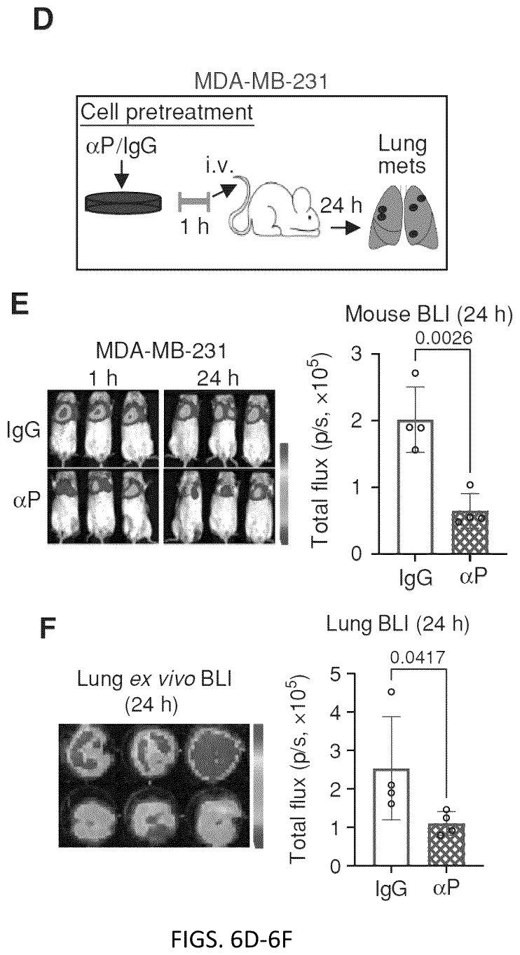

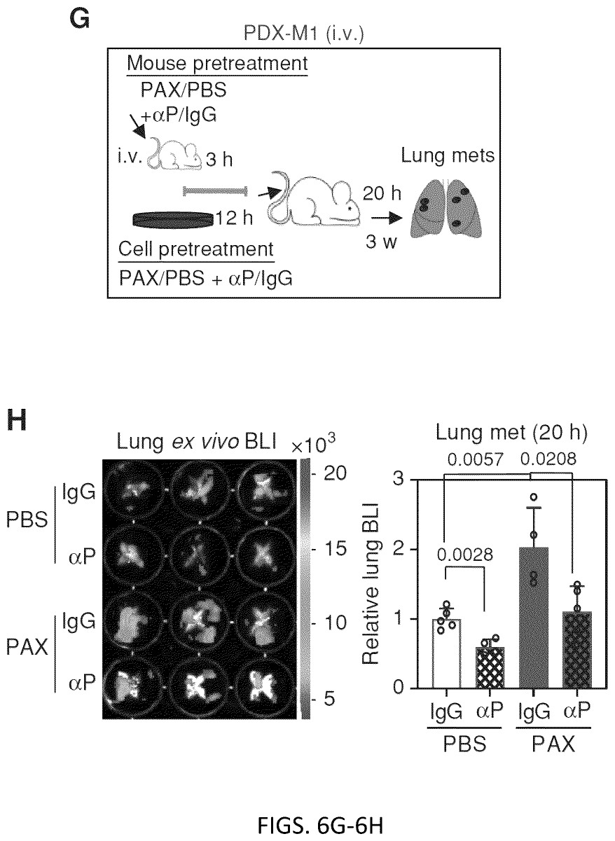

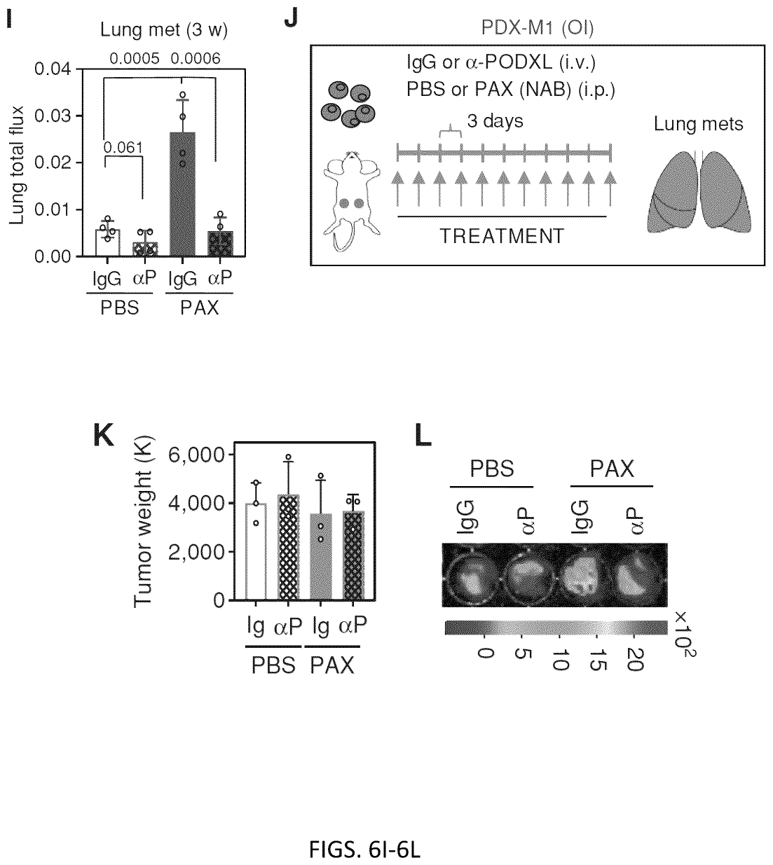

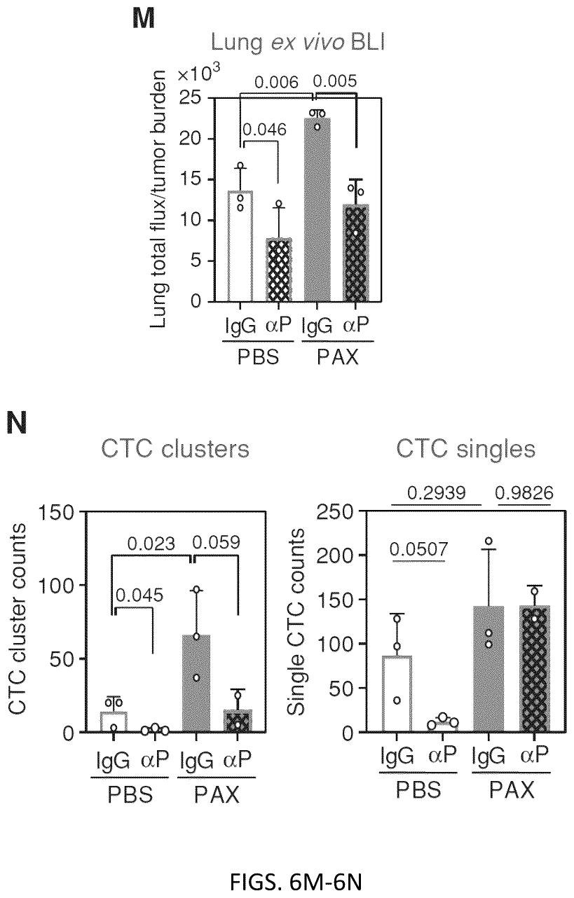

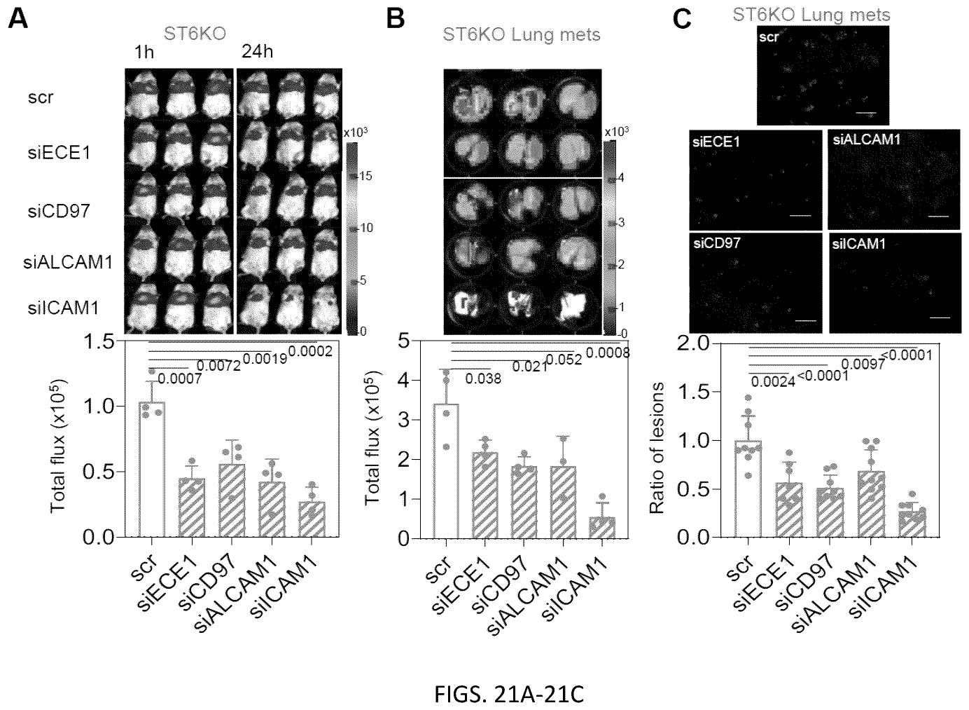

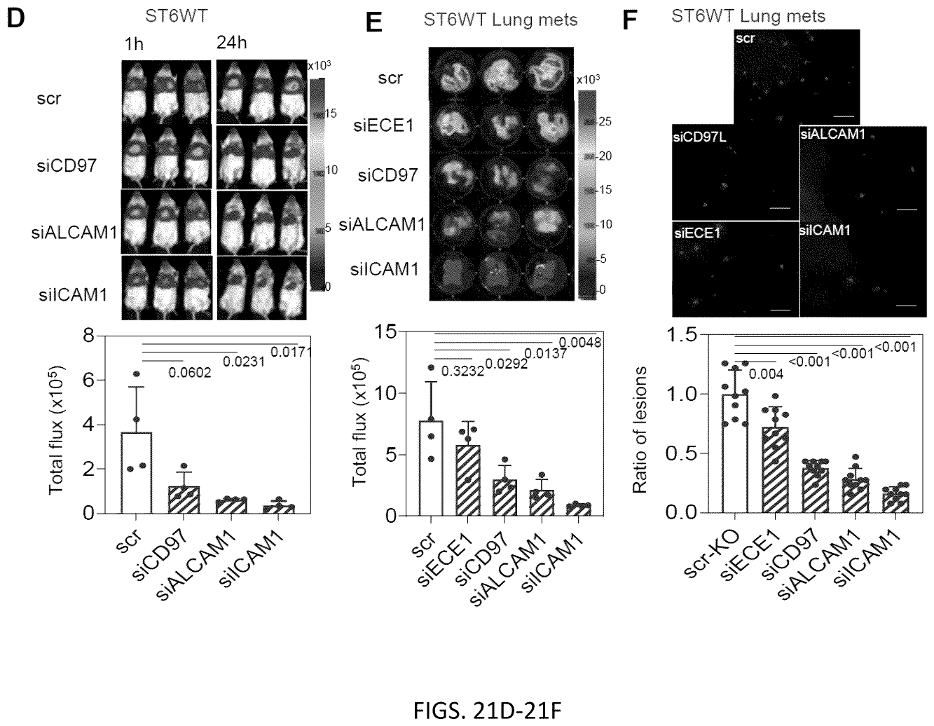

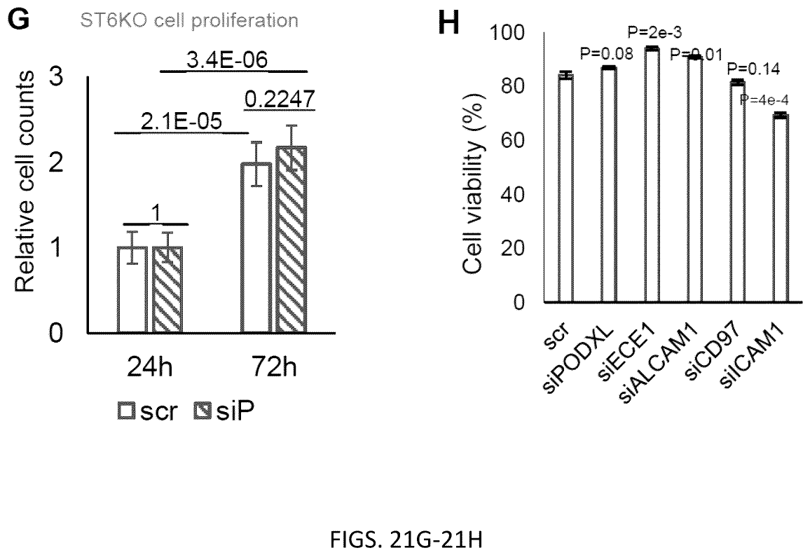

To demonstrate whether PODXL and other adhesion molecules contribute to ST6GAL1 deficiency-promoted metastatic seeding in vivo, we knocked down these genes in ST6KO and ST6WT cells prior to cell inoculation into mice. Reduced expression levels of PODXL, CD97, ECE1, ALCAM1, and ICAM1 in these tumor cells were verified via immunoblotting (FIGS. 5D and 20A). At 24 hours after tail-vein injection, PODXL knockdown effectively blocked 80% to 90% of metastatic seeding and tumor cluster formation in both ST6KO and ST6WT cells (FIGS. 6A-6C), similar to the inhibitory effects of ICAM1 knockdown (4, 15), whereas knockdown of ECE1, CD97, and ALCAM1 displayed relatively weaker inhibitory effects (FIGS. 21A-21F). PODXL knockdown had minimal effects on the proliferation or the viability of transfected cells (FIGS. 21G and 21H). Similarly, cell viability was not affected by CD97 knockdown and was slightly improved by knockdown of ECE1 or ALCAM1 except for a small decrease by ICAM1 downregulation (FIG. 21H).

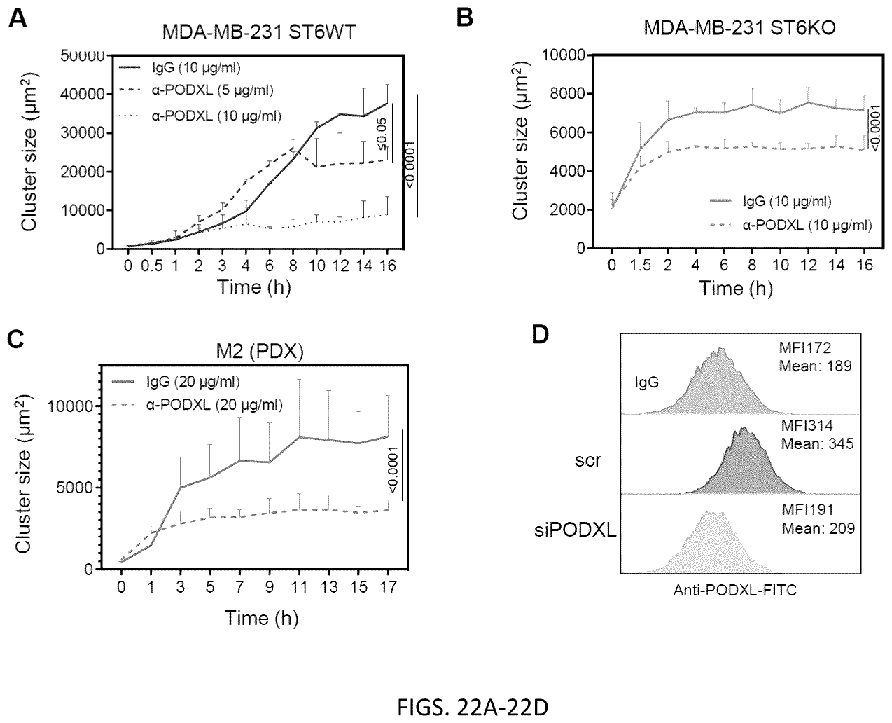

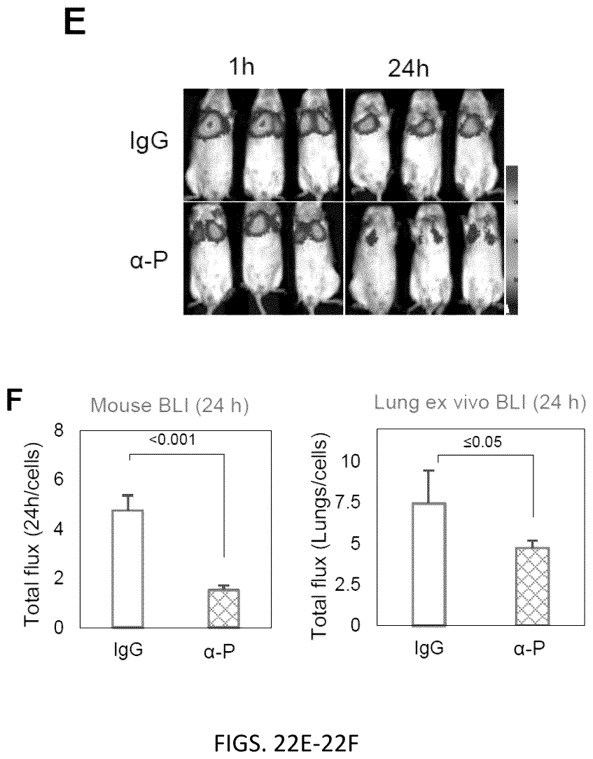

As a proof of concept, we investigated the possibility of targeting PODXL therapeutically using a neutralizing antibody in multiple tumor models. Administration with the anti-PODXL antibody (5-20 μg/mL) significantly blocked tumor cell cluster formation of ST6KO and ST6WT tumor cells (MDA-MB-231), as well as the M2 PDX cells ex vivo (FIGS. 22A-22C). Knockdown of PODXL diminished the binding of anti-PODXL to these cells, suggesting minimal off-target binding of this antibody to PODXL-negative cells (FIG. 22D). When anti-PODXL antibody was administered intravenously once (7 μg/mouse) at 1 hour prior to tumor cell inoculation, it efficiently blocked nearly half of the metastatic seeding from both ST6WT and ST6KO MDA-MB-231 tumor cells as measured 24 hours after tail-vein inoculation (FIGS. 6D-6F and 22E-22F).

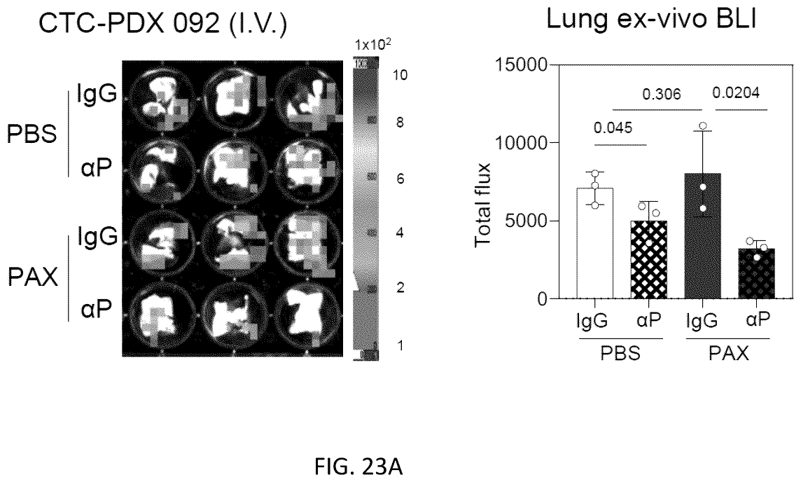

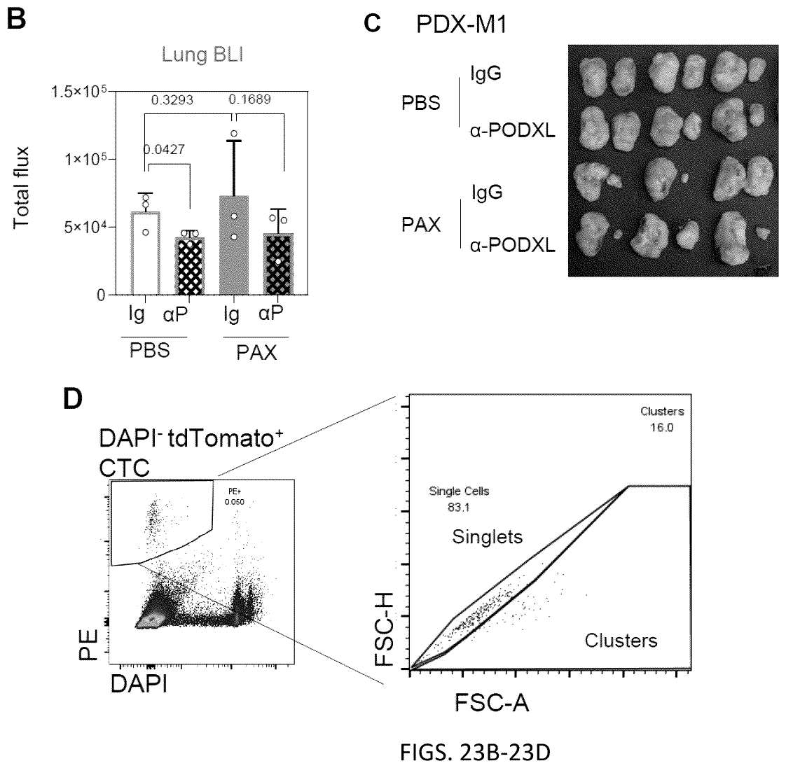

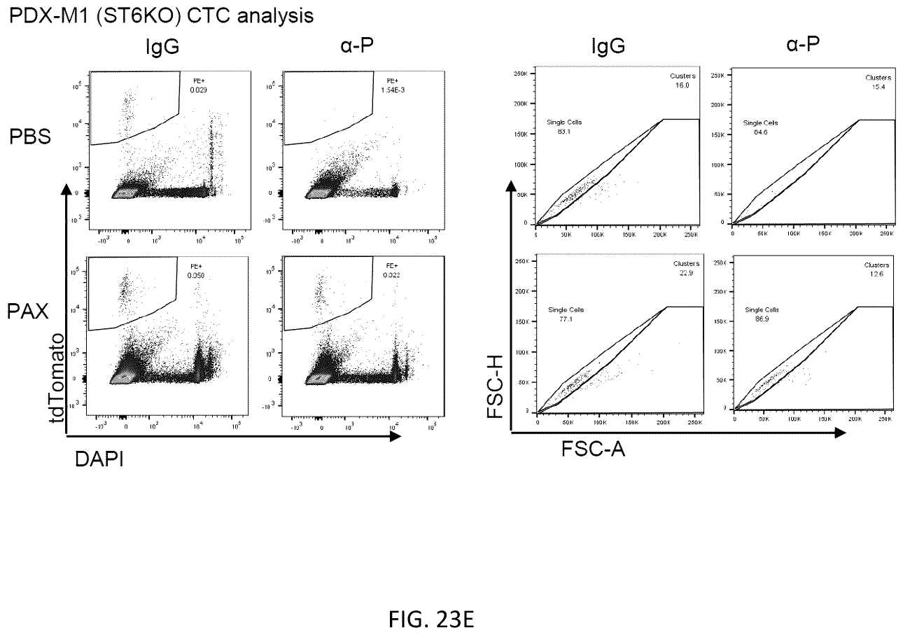

We then tested the efficacy of anti-PODXL in preventing and treating the metastatic PDX models (M1 and CTC-092), which resist PAX chemotherapy. In an experimental metastasis study, the dissociated ST6WT PDX tumor cells were pretreated with PAX (50 μg/mL) ex vivo for 12 hours and then inoculated via tail vein into the mice. Mice were pre-treated with PAX (27 mg/kg) 3 hours prior to cell inoculation (FIG. 6G). Revealed by the analyses at 20 hours and 3 weeks, PAX-promoted metastatic seeding of these tumor cells to the lungs was almost completely inhibited by concurrent treatment with anti-PODXL (20 μg/mL ex vivo and 30 μg/mouse via intravenous injection) in M1 PDX models (FIGS. 6G-6I). In CTC-092 PDX models, anti-PODXL administration also reduced lung colonization in both PBS and PAX treatment groups (FIG. 23A).

We further investigated the therapeutic effects of anti-PODXL on spontaneous metastasis that is elevated by ST6KO and PAX treatment in PDX tumor cells (FIG. 6I). Following orthotopic tumor implantations, the PAX administration (13.5 mg/kg, i.p., every 3 days for 11 cycles, started from the second week) failed to shrink the primary tumor but increased spontaneous lung metastases along with elevated CTC clusters (FIG. 6J-6N). Intravenous anti-PODXL treatment at the dosage of 20 μg/mouse concurrent to PAX treatment (every 3 days for 11 cycles) effectively blocked spontaneous metastases along with a dramatic reduction in CTC clusters (FIGS. 6N and 23B-23E).

In summary, our work reports PODXL as a promising new target of ST6GAL1, contributing to the increased formation of quiescent CTC clusters and breast cancer metastasis in response to PAX therapy.

### DISCUSSION

Collectively, our study demonstrates that PAX-induced loss of ST6GAL1 and α2,6-sialylation promotes adhesion molecule-mediated CTC cluster formation and metastatic seeding of TNBC. During chemotherapeutic treatment, the prognosis of TNBC is determined not only by treatment response in the primary tumor but also by the metastatic outgrowth. Chemoevasion is one of the most challenging problems compromising patient survival. PAX has previously been shown to delay tumor growth but increase metastasis with unknown mechanisms (38). Our study unveils the unexpected effects of chemoevasion and ST6GAL1 deficiency on promoting CTC cluster formation linked to an expected quiescence phenotype. A number of studies have also shown that PAX induces drug resistance through NF-κB activation and tumor cell dissemination by activating inflammation that promotes angiogenesis (39). PAX may also induce breast cancer cell intravasation and dissemination by promoting the formation of a microanatomic structure called tumor microenvironment of metastasis (known as TMEM; ref. 38). Interestingly, our signaling pathway analysis also reports that loss of ST6GAL1 results in activation of tumor-intrinsic inflammatory pathways that may also relate to chemoevasion and the responses in PAX-treated cells.

Targeting quiescent metastatic stem cells and/or CTC clusters is a demanding task to counter chemoevasion and block metastasis. The comprehensive glycoproteomic analysis of α2,6-sialylated proteins provides a robust platform by which we have further identified new adhesion protein substrates of ST6GAL1, such as PODXL, as innovative therapeutic targets. In addition to promoting homotypic CTC-CTC cluster formation, ST6GAL1 deficiency also promotes heterotypic tumor cell-endothelial cell interactions, thereby facilitating transendothelial migration, which is a gatekeeping step during metastatic seeding. Antibody-mediated blockade of PODXL interaction with neighboring cells further suggests a proof-of-concept therapeutic approach for inhibiting CTC cluster formation and blocking lung metastasis of TNBC.

Although PODXL is one of the top substrates responsible for ST6GAL1-mediated regulation in metastatic seeding, many others may be required for optimal tumor stem cell cluster formation, such as ICAM1 (15), CD44 (4), and EGFR (36), as demonstrated in our previous studies. Moreover, large CTC clusters may also be trapped in capillaries or enable closure of such vasculatures for initiation of colonization (16). Seeking combinational targeting approaches to prevent and treat metastasis remains urgent and to be further advanced.

Glycosylation of cell-surface proteins is constantly and dynamically changing and is associated with cancer development (18) in a context-dependent manner in various cancers, including breast (21), prostate (40), ovarian (41), and colorectal cancers (42, 43). Glycosylation patterns modulate cell-surface polarity and protein binding affinity to finely tune cell-cell and protein-protein interactions (18). Altered glycosyltransferases and glycosylation are often associated with oncogenic transformation in various cancers, including breast cancer. The most frequent altered glycosylations are sialylation, fucosylation, and O-glycan truncation (18, 19). N-glycan sialylation refers to the covalent addition of negatively-charged SAs via α2,3-, α2,6-, or α2,8-bonds to the terminal end of oligosaccharides that are linked to the asparagine (N) residue of glycoproteins (20).

Although sialylation may increase intercellular communication via lectin ligand-binding activities of glycoproteins (18, 19), loss of α2,6-sialylation promotes CTC cluster formation and metastatic seeding, likely through enhanced binding of ST6GAL1 substrate adhesion molecules. In addition to investigating a possible negative association between CD44 and ST6GAL1, future studies will be pursued to elucidate the biochemical properties and regulatory mechanisms underlying transient and dynamic loss of α2,6-sialylation in CTCs during metastasis and in response to chemotherapy. We speculate that the negative charges α2,6-SA brings to glycoproteins on cell surfaces may cause cell repulsion and shedding of migratory tumor cells at the early steps of metastasis, and removal of α2,6-SA in CTCs may expose strong binding sites among cell adhesion molecule and their ligands to facilitate clustering and seeding with advantageous quiescence to evade chemotherapy. In mammals, α2,6-sialylation is catalyzed by two different sialyltransferases, ST6GAL1 and ST6GAL2 (21). Although ST6GAL1 is ubiquitously expressed in human tissues and implicated in various tumors, including breast cancer, ST6GAL2 expression is limited to the brain cortex and embryos (21). It might be interesting to examine if chemotherapy or PAX impacts ST6GAL2 and cellular functions in those tissues, such as neuropathy.

### Methods

### Human Specimen Analyses

All human specimen collection and blood sample analyses were performed according to Northwestern University's IRB-approved protocol (IRB STU00203283) and following NIH guidelines for human subject studies. Written informed consent was provided by the patients whose blood and/or tissue specimens were analyzed for the study.

### OS Analysis

The cohort consisted of 157 patients with metastatic breast cancer longitudinally characterized for CTCs and CTC clusters at the Robert H. Lurie Comprehensive Cancer Center at Northwestern University (Chicago, IL). Patients were enrolled under the Investigator Initiated Trial NU16B06 (IRB STU00203283) at the time of initial metastasis or disease progression, independent of line of therapy. Imaging was performed according to the investigators' choice. Cluster enumeration was performed before treatment started (baseline) and at the first clinical evaluation one (E1) within a median of 3 months after the baseline time point. OS was defined as the time from baseline until any cause. Patients without an endpoint event at the last follow-up visit were censored. Differences were analyzed by the log-rank test and presented by Kaplan-Meier estimator plot.

### Animal Studies

PDX mouse models were kept in specific pathogen-free facilities in the Animal Resources Center at Northwestern University. All animal procedures complied with the NIH Guidelines for the Care and Use of Laboratory Animals and were approved by the Northwestern University Institutional Animal Care and Use Committee (IACUC protocol IS00016125). Animals were randomized by age. Sample sizes were specified based on the results of preliminary experiments. Immunodeficient NSG mice (8-10 weeks old) were utilized for ortho-topic implantation and tail-vein injection of multiple TNBC PDX models and MDA-MB-231 breast cancer cell lines. The PDX models were established as described previously (8).

### CTC Analyses by CellSearch and Flow Cytometry

Two complementary methods were previously established in the laboratory for CTC analyses in parallel using distinct blood sample tubes (4, 44). One is the FDA-approved CellSearch platform, with blood drawn directly into CellSave Preservative Tubes (Menarini Silicon Biosystems), which contain cell preservatives to keep CTCs intact under storage at room temperature. Another is flow cytometry of live CTCs (blood cell lineage-negative) with blood drawn into EDTA Vacutainer tubes (Becton Dickinson).

CellSearch. The CellSearch System (Menarini Silicon Biosystems) is semiautomated for blood sample processing, enrichment of EpCAM⁺ epithelial CTCs using the Epithelial Cell Kit (EpCAM-coated magnetic beads), and subsequent four-channel immunofluorescence staining as described by Cristofanilli and colleagues (44). Normally, CTCs are specified by combining three routine channels of DAPI positivity, negative for the white blood cell marker CD45, and positive for the epithelial marker CK (8, 18, 19), with the fourth and the only channel open for analysis of one customized candidate marker, such as glycans and proteins. Fluorophore-labeled lectins were used for glycan recognition, such as Flourescein-labeled Sambucus Nigra Lectin (SNA, EBL; Vector Laboratories, FL-1301) for α2,6-SA analysis. Fluorophore-conjugated antibodies were chosen for optimized detection of proteins, such as anti-PODXL (Santa Cruz Biotechnology, 3D3, sc-23904 PE) and anti-Ki-67 (Abcam, SP6, ab282173). For the PODXL staining, blood samples were prestained with antibody for 30 minutes at 37° C. and then proceeded for semiautomated processing on CellSearch as described (44, 45).

Flow Cytometry. The protocol of flow cytometry for CTC analysis was previously established (4) and optimized for comprehensive analysis of glycans and other protein markers. The blood samples were collected into EDTA Vacutainer tubes (Becton Dickinson) from patients with breast cancer and stored on ice or at 4° C. temporarily prior to manual blood processing within 24 hours. After multiple rounds (two to four rounds) of red blood cell lysis (lysis buffer, Sigma, R7757), white blood cells (mainly peripheral blood mononuclear cells) were washed twice with PBS. Cells were fixed with 3% paraformaldehyde for 30 minutes for glycan and protein profiling analyses. Cells were blocked with carbo-free blocking solution (Vector Laboratories, SP-5040) and stained with antibodies for proteins (CD45, epithelial marker EpCAM, and other blood cell lineage markers) and lectins for glycans, such as SNA-FITC, MALII-FITC, PHA-L-PE, LTL-FITC, RCA-Rho, LEL-APC, ConA-PE (Vector Laboratories), and MALII-FITC (GlycoMATRIX). CTCs were gated based on cell size (forward-scatter and side-scatter channels), CD45 negativity, and EpCAM^{+/-} CK^{+/-}.

### Cell Lines and Transfections

Human MDA-MB-231 cells and mouse breast cancer cell line 4T1 were purchased commercially from ATCC and periodically verified to be Mycoplasma-negative using the MycoAlert Mycoplasma Detection Kit (Lonza, LT07-218). Cell morphology, growth characteristics, and microarray gene expression analyses were compared with published information to ensure their authenticity. Early passage of cells (<15 passages) was maintained in Dulbecco's modified Eagle medium with 10% fetal bovine serum (FBS) plus 1% penicillin-streptomycin (P/S). Primary tumor cells were cultured in HuMEC-ready medium (Life Technologies) plus 5% FBS and 0.5% P/S in collagen type I (BD Biosciences)-coated plates. ON-TARGETplus siRNAs for ST6GAL1, PODXL, ALCAM1, ECE1, CD97, and ICAM1 and nontargeting control siRNA were purchased from Dharmacon and transfected using Dharmafect (Dharmacon) at 50 nmol/L; for the double knockdown, cells were transfected again after 24 to 48 hours. Transfection efficiency was evaluated by flow cytometry analysis and Western blotting.

### Tumor Dissociation and Orthotopic Injection

L2T- or L2G-labeled PDX tumors or MDA-MB-231 cell tumors were dissociated with liberase TH lysis buffer (Sigma-Aldrich) according to the supplied protocol, and after dissociation, cells were filtered with 70-μm and 40-μm strainers. Red blood cells were lysed with red blood cell lysing buffer (Sigma) for 1 minute on ice and washed with PBS. Next, 100 to 1,000 cells were resuspended in Matrigel:phosphate-buffered saline (PBS; 50:50 μL) and injected under the mammary fad pad. Tumor growth was monitored weekly under in vivo bioluminescence imaging (see below). After mice were euthanized, tumors were dissected and weighed and dissociated for clustering assays, Western blotting, and flow cytometry analysis. Lung metastases were imaged by fluorescence microscopy and bioluminescence imaging and fixed with 4% paraformaldehyde for immunofluorescence staining and 4% formalin for IHC. Lungs were dissociated using the same techniques as tumors for flow cytometry analysis to quantify lung metastasized cells.

### Tail-Vein Injection

After dissociation of the tumors, L2G- or L2T-labeled PDX cells (0.5×10⁵-2×10⁵/mouse) or MDA-MB231 cells (1×10⁵/mouse) were resuspended in PBS (200 L/mouse) and reinjected via the tail vein. Because the bioluminescence signal is dependent on cellular metabolic energy and modulation of the ST6GAL1 gene affects cellular activity, the same number of cells per mouse was imaged by bioluminescence before injection to estimate lung localization of the cells. Mice were imaged right after tail-vein injection and 24 hours after injection. After that, mice were euthanized and lung metastasis was measured by bioluminescence imaging and fluorescence microscopy. Lungs were dissociated for flow cytometry analysis and fixed for immunofluorescence and IHC analysis.

PAX Treatment of M1-PDX Model after Orthotopic Inoculation

ST6WT cells (1×10⁶) were injected into a mammary fad pad. After 1 week of tumor growth, mice were treated with PAX-NAB (13.5 mg/kg) or PBS control (every 3 days, 10 times). Mice were sacrificed, and blood was collected from the heart individually. Red blood cells were lysed by using RBC buffer (0.15 M ammonium chloride, 1 mmol/L potassium bicarbonate, 0.1 mmol/L EDTA, pH 7.2-7.4). tdTomato-labeled PDX-M1 CTCs in blood and the percentage of SNA-high and Ki-67 staining in single CTCs and CTC clusters were quantified using flow cytometry. The gating strategy of CTCs is shown in FIG. 23. After collecting blood, tumor and lungs were dissected and lung ex vivo bioluminescence imaging was measured as explained above.

### Anti-PODXL Antibody Treatment of MDA-MB-231 Tumor Cells Via Tail-Vein Injection

ST6WT and ST6KO cells (6.5×10⁴) were pretreated with 7 μg/mouse concentration of antibody or isotope IgG control for 2 hours at 37° C. Cells in 200 μL were then inoculated directly to the mice via the tail vein. Because bioluminescence imaging of the ST6WT and ST6KO cells was different due to cell activity, we first performed bioluminescence imaging of the same number of cells for further analyses. After cell inoculation, bioluminescence imaging of the mice was performed after 1 hour and 24 hours and then the mice were sacrificed. Lung ex vivo images were taken right after the sacrifice. The quantifications of the total fluxes were normalized with the total flux of the cells that were imaged before cell tail-vein injection.

### Anti-PODXL and PAX-NAB Treatment of M1-PDX/CTC-092-PDX Via Tail-Vein Injection

Luc2-tdTomato-labeled PDX-M1 or CTC-092-PDX cells were dissociated and pretreated with PAX-NAB (50 μg/mL) and/or anti-PODXL antibody or isotope control IgG (20 μg/mL) for 12 hours. After washing and centrifugation, 5×10⁵ cells were inoculated into the tail vein of the mice pretreated with PAX (27 mg/kg) and anti-PODXL or IgG (˜30 μg/mouse) 3 hours prior to cell inoculation. Twenty hours or 3 weeks after tumor cell injections, bioluminescence signals of the mice and dissected lungs were imaged using SII LAGO (excitation wavelength was 465 nm and emission filter wavelength was 510 nm).

Cotreatment of Anti-PODXL and PAX in M1-PDX after Orthotopic Inoculation

ST6KO cells (1×10⁶) were injected into a mammary fad pad. After 1 week of tumor growth, mice were treated with PAX-NAB (13.5 mg/kg) and/or anti-PODXL antibody or isotope control IgG (10 μg/mouse, every 3 days, 12 times). Mice were sacrificed, and blood was collected from the heart individually. Red blood cells were lysed using RBC buffer (0.15 M ammonium chloride, 1 mmol/L potassium bicarbonate, 0.1 mmol/L EDTA, pH 7.2-7.4). tdTomato-labeled PDX-M1 CTCs in blood were quantified using flow cytometry. The gating strategy is shown in FIG. 20. After blood collection, tumor and lungs were dissected and lung ex vivo bioluminescence imaging was measured as explained above. The total flux of lungs was normalized with tumor weight.

### Bioluminescence Imaging

After intraperitoneal injections with 100 μL of D-luciferin (30 mg/mL, Gold Biotechnology, 115144-35-9), mice were anesthetized with isoflurane. Bioluminescence images were acquired using a Spectral Lago system from Spectral Instruments Imaging, and the signals are presented as total flux (photons/second, p/s) and fold changes of the total flux signals (Aura, version 2.2.1.1). Acquisition times ranged from 5 seconds to 5 minutes.

### Flow Cytometry and Cell Sorting

L2T- or L2G-labeled PDX tumor cells or MDA-MB-231 cells were washed twice with PBS and blocked with carbo-free blocking solution (Vector Laboratories, SP-5040) for 20 minutes on ice (1×10⁶ cells in 0.2 mL). Then cells were incubated with the appropriate lectins (1 L/1×10⁶ cells), including biotinylated SNA, MAL-II, LTL, or PHA-L (Vector Laboratories). Streptavidin-labeled APC antibodies were used as secondary antibodies at the beginning of the experiments, and later SNA-FITC and SNA-APC (Vector Laboratories) were used at the same concentrations. CD44 antibody with PE, APC, FITC, or Pe-Cy7 labels (R&D Systems) was coincubated with lectins at the concentrations suggested by the manufacturers. Cells were analyzed with a BD-LSR II flow cytometer (BD Biosciences) or a BDAria cell sorter (BD Biosciences). DAPI was used for cell viability control.

### Western Blotting and Immunoprecipitation

Cultured cells were scraped and washed twice with PBS before lysing with RIPA buffer (Thermo Fisher) with protease inhibitor cocktail (1:100 dilution, Thermo Fisher). Lysis was completed for 30 minutes on ice, and the mixture was centrifuged for 10 minutes at 4° C. and 14,000 rpm. For Western blotting, 2 to 10 μg of protein was denatured at 100° C. for 5 minutes and subjected to SDS-PAGE and then transferred to PVDF membranes. For immunoprecipitation, 100 to 1,000 μg of protein was first preincubated with agarose beads (Vector Laboratories) for 1 hour. Then SNA-tagged agarose beads (Vector Laboratories, Al-1303) were added to the lysates and incubated overnight at 4° C. on a rotator. SNA-bound proteins were eluted with SDS buffer (Bio-Rad) and subjected to SDS-PAGE. Antibodies against ST6GAL1 (R&D Systems, AF5924), ST3GAL1 (R&D Systems, AF6905-SP), FUT3 (I; anti-CD174, BioLegend, 392602), FUT3 (II; Abcam, ab110082), CERCAM (Santa Cruz Biotechnology, D-4, sc-514083), Serpine-1 (Proteintech, 13801-1-AP), L1CAM (Thermo Fisher, 1.G11B1, MA526429), CD44 (Thermo Fisher, 8E2F3, MA515462), PODXL (Santa Cruz Biotechnology, 3D3, sc-23904), ICAM1 (Sigma, HPA004877), ECE1 (Santa Cruz Biotechnology, A-6, sc-376017), ALCAM1 (Proteintech, 21972-1-AP), CD97 (Santa Cruz Biotechnology, G-8, sc-166852), Na+/K□+ ATPase aptha1 (Santa Cruz Biotechnology, C464.6), and β-actin (Sigma, A5441) were used as primary antibodies, and horseradish peroxidase (HRP)-conjugated secondary antibodies were from Promega (Rabbit W401B and Mouse W402B). The substrate ECL was detected by Pierce ECL2 solution (Thermo Fisher Scientific, 1896433A).

Release of N-Linked Glycans from Cells

About 20×10⁶ cells were lysed in a high-salt buffer (2M NaCl, 5 mmol/L EDTA in 100 mmol/L Tris-HCl, pH 7.5) and centrifuged at 15,000 rcf for 5 minutes, and the pellet was collected. The protein pellet was subsequently dissolved in a urea lysis buffer (8 mol/L urea, 4% CHAPS, 100 mmol/L DTT, 5 mmol/L EDTA in 100 mmol/L Tris-HCl, pH 8) and heated at 60° C. for 45 minutes for protein reduction. Further, 300 mmol/L iodoacetamide was added to the protein sample and incubated in the dark at room temperature for 45 minutes. The samples were desalted, and to make 500 μL of the sample, 50 μL of 10× New England Biolabs (NEB) glycobuffer 2 buffer was added to prepare a 1× solution. The samples were then treated with 5 μL of PNGase F (NEB), and the mixture was incubated at 37□C for 16 hours to release the N-linked glycans. The N-glycans were recovered by passing the mixture through a C18 cartridge, eluting the N-glycans with 5% acetic acid, and lyophilization. The N-glycans were permethylated by methyl iodide in the presence of sodium hydroxide-dimethyl sulfoxide (NaOH-DMSO) and further analyzed by ESI-MSn (46).

### ESI-MSn Analysis of Glycans

About 2 μL of permethylated glycans was dissolved in 98 μL of ESI-MS infusion buffer (1:1:1 methanol, 0.1% formic acid in water and acetonitrile) and infused into an Orbitrap Fusion Tribrid mass spectrometer. A precursor scan was acquired at 120,000 resolution, and subsequent collision-induced dissociation (CID) MSn was acquired for the detailed structural characterization of glycans. For the determination of sialic acid linkages, the glycans were infused with lithium salts for increased cross-ring fragmentation of glycans. The cross-ring fragments of the partially methylated galactose to which the SAs are either 2,3- or 2,6-linked are different and allowed the identification of sialic acid linkages (46).

### Glycoproteomics

The glycoproteins were enriched by SNA lectin from membrane fractions as described in the “Western Blotting and Immunoprecipitation” section. Glycoproteins were eluted by boiling in an SDS buffer. The control and knockout samples were run on SDS gel and stained with Coomassie Blue; the gel bands were excised, destained, reduced, and alkylated; and proteins were digested by trypsinization at 37° C. for 24 hours. Further, the digested peptides were extracted from the gel pieces, filtered, and evaporated to dryness. The peptides were redissolved in 0.1% formic acid and injected into an Orbitrap Fusion Tribrid mass spectrometer coupled with a Dionex LC system for glycoproteomics analysis. An Acclaim PepMap nano-LC column (Thermo Scientific; 164568) of 150 mm length with 75-μm internal diameter (id), filled with 3 μm, 100 Å C18 material (reverse phase), was used for chromatographic separation of peptides. The mass spectrometer method was set up in data-dependent acquisition mode with an MS1 automatic gain control target value of 5×10⁵. After the precursor ion scan at 120,000 resolutions in the Orbitrap analyzer, intense precursors were selected for subsequent fragmentation using higher-energy collision dissociation (HCD) or CID (product-triggered based on the presence of glycan oxonium ions in the HCD) within 3 seconds at a normalized collision energy of 28 and 35, respectively. For internal mass calibration, 445.120025 ion was used as lock mass with a target lock mass abundance of 0%. Charge state screening was enabled, and precursors with an unknown charge state or a charge state of +1 were excluded. Dynamic exclusion was enabled (exclusion size list 100, exclusion duration 30 s). The fragment ions were analyzed in the Orbitrap at 15,000 resolution. The LC-MS/MS chromatogram of control and knockout was analyzed by searching against the human protein database using Byonic 2.3.5 software with variable modifications such as carbamidomethylation of cysteine and oxidation of methionine. Trypsin was chosen as the digestion enzyme, and the human N-glycan database was used for the glycopeptide search. Only glycopeptides with higher scores and good MS/MS fragment ion presence were considered while annotating the proteins. Glycoproteomic data were deposited in glycopost.glycos-mos.org with the accession number GPST000273.0 (pin code: 1613).

### Cell Clustering Assay

Dissociated PDX primary tumor cells in single-cell suspension were seeded in 96-well plates coated with collagen type I. MDA-MB-231-derived cells were detached with EDTA buffer (0.05 mmol/L), and single cells were seeded in suspension in 96-well plates pretreated with poly-hydroxyethyl methacrylate (Poly-HEMA, Sigma-Aldrich). For the NA treatment, the same number of cells were treated with NA at 0, 10, 50, and 100 mU/mL concentrations. The cells were then incubated and monitored by the IncuCyte live-cell imaging system (Essen BioScience), and images were acquired every 2 hours. Cluster size was analyzed over time by the IncuCyte ZOOM software. In addition, MDA-MB-231 tumor cells were incubated with the anti-PODXL antibody for 30 minutes on ice and imaged using the same experimental method.

### Cell Growth Competition Assay

Equal numbers of L2T-ST6WT and L2G-ST6KO or L2G-ST6WT and L2T-ST6KO cells were cultured on the same plate. Cells were counted with flow cytometry on days 0, 6, 18, 25, and 31. Before cell detachment, cells were imaged under a fluorescence microscope. Cells were passaged when they reached 70% to 80% confluence.

### Colony Formation Assay

ST6WT and ST6KO cells were cultured in 4-well chambers or 6-well plates (2×10³ cells) and incubated for a week. Cell cultures were washed gently with PBS and fixed with methanol (25 minutes, 4° C.). Next, cells were stained with crystal violet:methanol at the ratio of 1:1 (Sigma-Aldrich and Sigma, respectively). The number and size of the colonies were determined using ImageJ.

### Quiescence Assay

ST6WT or ST6KO cells were washed twice with PBS. Cells were stained with pyronin Y (Sigma-Aldrich) according to the manufacturer's protocol. Briefly, 1×10⁶ cells were resuspended in 1 mL of PBS containing 2% FBS and 1 μL of pyronin Y solution was added. Cells were then incubated at 37° C. for an hour. Then, Hoechst 33342 was added and the cells were incubated for an additional hour. Cells were washed twice with pyronin Y and Hoecsht containing PBS and then stained with lectins or antibodies if necessary or directly loaded onto the flow cytometer.

### Cell Viability and Proliferation Assay

To track cell proliferation, a CellTrace CFSE cell proliferation kit (Thermo Fisher) was used. Briefly, equal numbers of ST6WT and ST6KO cells were resuspended in CellTrace CFSE solution (1:1,000 dilution) and incubated for 20 minutes at 37° C. in the dark. The complete medium was added to a total volume of 10 mL and further incubated for 5 minutes. Cell suspensions were pelleted and replated on 10-cm plates, and proliferation was analyzed after 24 hours by flow cytometry. For the cell viability assay, because ST6WT and ST6KO cell proliferation rates are different, ST6WT and ST6KO cells were seeded at a ratio of 1:4 in 10-cm plates and grown to a similar confluency on the treatment day. When cells reached 80% confluency, cells were treated with paclitaxel (Sigma) at 0, 10, 25, 50, 100, and 250 μg/mL concentration. Cell viability was measured 24 hours after treatment with DAPI by flow cytometry analysis. To observe time-dependent resistance to paclitaxel, we treated cells with 25 μg/mL paclitaxel and harvested them 0, 8, and 24 hours after treatment. For the treatment of the CTC-PDX-092 model, cells were treated with PAX-NAB at 0, 25, 50, 100, and 250 μg/mL concentrations, and cell death in response to PAX-NAB was measured using DAPI staining by flow cytometry. After SNA sorting (−high and −low), cells were treated with PAX (25 μg/mL) or left untreated and 24 hours after treatment, cellular viability was measured as mentioned above.

### Gene Modulation

L2G and L2T dual optical reporter-labeled MDA-MD-231 cells, 4T1 cells, and PDX models were generated as described previously. The CRISPR-Cas9 technique was used to generate ST6KO cells. The gRNA and Cas9-coexpressing lentiviral plasmid targeting human ST6GAL1 gRNA-1 5′ CACCGTCCTACAAGGGGCCGGGACC (SEQ ID NO: 1) and gRNA-2 5′ CACCGCATTCACGTGGTCTCGAAGG (SEQ ID NO: 2) and control non-target gRNA were generated by Dr. Derek Abbott (request contact dwa4@case.edu) and kindly gifted by Dr. Brian A. Cobb, Case Western Reserve University. Mouse ST6GAL1-targeting gRNAs, control nontargeting gRNA, and Cas9-expressing plasmids were purchased from Sigma. L2G- or L2T-labeled MDA-MB-231 cells, 4T1 cells, or PDX models were coinfected with appropriate lentiviruses (3 particle-forming units/cell). After 48 hours of infection, cells were selected with puromycin (3 μg/mL). Then cells were sorted for SNA-negative cells. MDA-MB-231 and 4T1 cells were sorted into single cells and plated on 96-well plates to grow single cell-derived clones. For the ST6GAL1-overexpressing cells (ST60E), full-length ST6GAL1 was inserted into a pEZ-Lv122 vector purchased from GeneCopoeia (EX-M03510Lv122). Virus infection and selection of the cells were performed as described above.

### RNA-Seq

Total RNA was isolated from ST6WT and ST6KO MDA-MB-231 cells using TRIzol (Thermo Fisher Scientific) according to the manufacturer's protocol. RNA-seq was performed at the Center for Genetic Medicine Sequencing Core Facility, Northwestern University. RNA-seq was performed on a HiSeq 4000, and a library was made using a TruSeq Total RNA-Seq Library Prep Kit. Data were processed and quantified using STAR (47), DESeq2 (48), and HTSeq (49). Differentially expressed genes were defined by cutoffs of false discovery rate<0.05 and log₂ (fold change)>0.48 or <−0.48. Finally, the pathway analysis of significantly differentially expressed genes was obtained using Metascape (http://metascape.org; ref. 50). The data are accessible at the Gene Expression Omnibus (GEO) with accession number GSE174080 (release date: Dec. 1, 2022).

### Cell-Binding Assay

MDA-MB-231 cells (ST6WT and ST6KO) were plated in 96 wells/plate (2×10⁴ cells/well) and cultured overnight, and the next day after cells were attached completely, cells were washed with PBS twice. On the other hand, membranes of the ST6WT and ST6KO cells were fractionated according to the protocol indicated above. Endogenous PODXL was pulled down using an anti-PODXL antibody (Santa Cruz Biotechnology, 3D3, sc-23904). Glycine (0.1 mol/L, pH 2.5) was utilized for antibody/antigen (anti-PODXL/PODXL) release from agarose beads. Acidic condition was neutralized immediately by adding Tris-HCl (1 mol/L, pH 8.8). Purified complexes were then labeled with HRP (EZ-Link plus activated peroxidase kit, Thermo Fisher, 31489) according to the provided protocol. HRP-labeled purified PODXL from ST6WT and ST6KO cells was added onto the cells and incubated for 1 to 2 hours at 37° C. Cell-protein complexes were then washed with PBS 5 times. TMB substrate solution (Thermo Scientific, N301) and stop solution (Thermo Scientific, N600) were used to visualize the protein binding to the cells (absorbance were measured at 450 nm).

### Transendothelial Cell Migration

Transendothelial cell migration of cancer cells through HUVECs was quantified as described previously (35, 51). Briefly, HUVECs were grown on hydrated collagen gels until confluent. ST6WT and ST6KO MDA-MB-231 cells were added to the medium. The cells were allowed to transmigrate for 3 hours at 37° C. Then, the suspending cells were washed with PBS and plates were fixed with 10% neutral buffered formalin. Cells on the top of the HUVEC and the cells that migrated through HUVECs were counted.

### Quantification of Total Intensity of CellSearch Images

The images of the individual cells collected by CellSearch analysis were analyzed using ImageJ software manually. The mean intensity was multiplied by the total area to find the total intensity of DAPI. The cutoff value of the binary assessment of Ki-67-negative cells was <75. Cell-cycle determination using DAPI intensity was as follows: G₁/G₀≤100.000, G₂-M 100.001-139.999, and S phase≥140.000. The correlation of DAPI versus Ki-67/SNA was calculated using www.socscistatistics.com.

### Statistical Analysis

Categorical variables were reported as frequency distributions, whereas continuous variables were described through median and interquartile ranges. Matched pair variations of cluster enumerations were tested across baseline and E1 through the Wilcoxon signed rank test. Patient data analysis was conducted using StataCorp 2019 Stata Statistical Software (Release 16.1), R (version 4.1.0, The R foundation for Statistical Computing), and JMP (version 16, SAS Institute). Microsoft Excel and GraphPad Prism were used to perform Student t test and calculate P values for all in vitro assays and analyses unless specified otherwise. P<0.05 was considered statistically significant and is represented with * for P<0.05, ** for P<0.01, *** for P<0.001, and **** for P<0.0001. Data are presented as mean±standard deviation (SD) unless specified otherwise.

### REFERENCES

1. Cheung K J, Padmanaban V, Silvestri V, Schipper K, Cohen J D, Fairchild A N, et al. Polyclonal breast cancer metastases arise from collective dissemination of keratin 14-expressing tumor cell clusters. Proc Natl Acad Sci USA 2016; 113:E854-63.
2. Mu Z, Wang C, Ye Z, Austin L, Civan J, Hyslop T, et al. Prospective assessment of the prognostic value of circulating tumor cells and their clusters in patients with advanced-stage breast cancer. Breast Cancer Res Treat 2015; 154:563-71.
3. Aceto N, Bardia A, Miyamoto D T, Donaldson M C, Wittner B S, Spencer J A, et al. Circulating tumor cell clusters are oligoclonal precursors of breast cancer metastasis. Cell 2014; 158:1110-22.
4. Liu X, Taftaf R, Kawaguchi M, Chang Y F, Chen W, Entenberg D, et al. Homophilic CD44 interactions mediate tumor cell aggregation and polyclonal metastasis in patient-derived breast cancer models. Cancer Discov 2019; 9:96-113.
5. Zoller M. CD44: can a cancer-initiating cell profit from an abundantly expressed molecule? Nat Rev Cancer 2011; 11:254-67.
6. Vagia E, Mahalingam D, Cristofanilli M. The landscape of targeted therapies in TNBC. Cancers (Basel) 2020; 12:916.
7. Foulkes W D, Smith I E, Reis-Filho J S. Triple-negative breast cancer. N Engl J Med 2010; 363:1938-48.
8. Liu H, Patel M R, Prescher J A, Patsialou A, Qian D, Lin J, et al. Cancer stem cells from human breast tumors are involved in spontaneous metastases in orthotopic mouse models. Proc Natl Acad Sci USA 2010; 107:18115-20.
9. Gianni L, Baselga J, Eiermann W, Guillem Porta V, Semiglazov V, Lluch A, et al. Feasibility and tolerability of sequential doxorubicin/paclitaxel followed by cyclophosphamide, methotrexate, and fluorouracil and its effects on tumor response as preoperative therapy. Clin Cancer Res 2005; 11:8715-21.
10. Kalinsky K, Barlow W E, Gralow J R, Meric-Bernstam F, Albain K S, Hayes D F, et al. 21-Gene assay to inform chemotherapy benefit in node-positive breast cancer. N Engl J Med 2021; 385:2336-47.
11. Gianni L, Baselga J, Eiermann W, Porta V G, Semiglazov V, Lluch A, et al. Phase III trial evaluating the addition of paclitaxel to doxorubicin followed by cyclophosphamide, methotrexate, and fluorouracil, as adjuvant or primary systemic therapy: European Cooperative Trial in Operable Breast Cancer. J Clin Oncol 2009; 27:2474-81.
12. Rastogi P, Anderson S J, Bear H D, Geyer C E, Kahlenberg M S, Robidoux A, et al. Preoperative chemotherapy: updates of National Surgical Adjuvant Breast and Bowel Project Protocols B-18 and B-27. J Clin Oncol 2008; 26:778-85.
13. Miles D, Gligorov J, Andre F, Cameron D, Schneeweiss A, Barrios C, et al. Primary results from IMpassion131, a double-blind, placebocontrolled, randomised phase III trial of first-line paclitaxel with or without atezolizumab for unresectable locally advanced/metastatic triple-negative breast cancer. Ann Oncol 2021; 32:994-1004.
14. Cortes J, Cescon D W, Rugo H S, Nowecki Z, Im S A, Yusof M M, et al. Pembrolizumab plus chemotherapy versus placebo plus chemotherapy for previously untreated locally recurrent inoperable or metastatic triple-negative breast cancer (KEYNOTE-355): a randomised, placebo-controlled, double-blind, phase 3 clinical trial. Lancet 2020; 396:1817-28.
15. Taftaf R, Liu X, Singh S, Jia Y, Dashzeveg N K, Hoffmann A D, et al. ICAM1 initiates CTC cluster formation and trans-endothelial migration in lung metastasis of breast cancer. Nat Commun 2021; 12:4867.
16. Schuster E, Taftaf R, Reduzzi C, Albert M K, Romero-Calvo I, Liu H. Better together: circulating tumor cell clustering in metastatic cancer. Trends Cancer 2021; 7:1020-32.
17. Ramos E K, Tsai C F, Jia Y, Cao Y, Manu M, Taftaf R, et al. Machine learning-assisted elucidation of CD81-CD44 interactions in promoting cancer stemness and extracellular vesicle integrity. Elife 2022; 11: e82669.
18. Pinho S S, Reis C A. Glycosylation in cancer: mechanisms and clinical implications. Nat Rev Cancer 2015; 15:540-55.
19. Reily C, Stewart T J, Renfrow M B, Novak J. Glycosylation in health and disease. Nat Rev Nephrol 2019; 15:346-66.
20. Li F, Ding J. Sialylation is involved in cell fate decision during development, reprogramming and cancer progression. Protein Cell 2019; 10: 550-65.
21. Garnham R, Scott E, Livermore K E, Munkley J. ST6GAL1: a key player in cancer. Oncol Lett 2019; 18:983-9.
22. Jones R B, Dorsett K A, Hjelmeland A B, Bellis S L. The ST6Gal-I sialyltransferase protects tumor cells against hypoxia by enhancing HIF-lalpha signaling. J Biol Chem 2018; 293:5659-67.
23. Swindall A F, Bellis S L. Sialylation of the Fas death receptor by ST6Gal-I provides protection against Fas-mediated apoptosis in colon carcinoma cells. J Biol Chem 2011; 286:22982-90.
24. Britain C M, Holdbrooks A T, Anderson J C, Willey C D, Bellis S L. Sialylation of EGFR by the ST6Gal-I sialyltransferase promotes EGFR activation and resistance to gefitinib-mediated cell death. J Ovarian Res 2018; 11:12.
25. Smerage J B, Barlow W E, Hortobagyi G N, Winer E P, Leyland-Jones B, Srkalovic G, et al. Circulating tumor cells and response to chemotherapy in metastatic breast cancer: SWOG S0500. J Clin Oncol 2014; 323483-9.
26. Tsai C F, Zhang P, Scholten D, Martin K, Wang Y T, Zhao R, et al. Surfactant-assisted one-pot sample preparation for label-free singlecell proteomics. Commun Biol 2021; 4:265.
27. Dorsett K A, Jones R B, Ankenbauer K E, Hjelmeland A B, Bellis S L. Sox2 promotes expression of the ST6Gal-I glycosyltransferase in ovarian cancer cells. J Ovarian Res 2019; 12:93.
28. Schultz M J, Holdbrooks A T, Chakraborty A, Grizzle W E, Landen C N, Buchsbaum D J, et al. The tumor-associated glycosyltransferase ST6Gal-I regulates stem cell transcription factors and confers a cancer stem cell phenotype. Cancer Res 2016; 76:3978-88.
29. Swindall A F, Londono-Joshi A I, Schultz M J, Fineberg N, Buchsbaum D J, Bellis S L. ST6Gal-I protein expression is upregulated in human epithelial tumors and correlates with stem cell markers in normal tissues and colon cancer cell lines. Cancer Res 2013; 73:2368-78.
30. Johnson D G, Schwarz J K, Cress W D, Nevins J R. Expression of transcription factor E2F1 induces quiescent cells to enter S phase. Nature 1993; 365:349-52.
31. Kwon J S, Everetts N J, Wang X, Wang W, Della Croce K, Xing J, et al. Controlling depth of cellular quiescence by an Rb-E2F network switch. Cell Rep 2017; 20:3223-35.
32. Poppy Roworth A, Ghari F, La Thangue N B. To live or let die—complexity within the E2F1 pathway. Mol Cell Oncol 2015; 2:e970480.
33. Nagy A, Lanczky A, Menyhart O, Gyorffy B. Validation of miRNA prognostic power in hepatocellular carcinoma using expression data of independent datasets. Sci Rep 2018; 8:9227.
34. Weber E W, Han F, Tauseef M, Birnbaumer L, Mehta D, Muller W A. TRPC6 is the endothelial calcium channel that regulates leukocyte transendothelial migration during the inflammatory response. J Exp Med 2015; 212:1883-99.
35. Muller W A, Weigl S A, Deng X, Phillips D M. PECAM-1 is required for transendothelial migration of leukocytes. J Exp Med 1993; 178:449-60.
36. Liu X, Adorno-Cruz V, Chang Y F, Jia Y, Kawaguchi M, Dashzeveg N K, et al. EGFR inhibition blocks cancer stem cell clustering and lung metastasis of triple negative breast cancer. Theranostics 2021; 11:6632-43.
37. Cherian S, Potdar V, Jadhav S, Yadav P, Gupta N, Das M, et al. SARSCoV-2 spike mutations, L452R, T478K, E484Q and P681R, in the second wave of COVID-19 in Maharashtra, India. Microorganisms 2021; 9:1542.
38. Karagiannis G S, Pastoriza J M, Wang Y, Harney A S, Entenberg D, Pignatelli J, et al. Neoadjuvant chemotherapy induces breast cancer metastasis through a TMEM-mediated mechanism. Sci Transl Med 2017; 9:eaan0026.
39. Dong Q G, Sclabas G M, Fujioka S, Schmidt C, Peng B, Wu T, et al. The function of multiple IkappaB: NF-kappaB complexes in the resistance of cancer cells to Taxol-induced apoptosis. Oncogene 2002; 21:6510-9.
40. Wei A, Fan B, Zhao Y, Zhang H, Wang L, Yu X, et al. ST6Gal-I overexpression facilitates prostate cancer progression via the PI3K/Akt GSK-3beta/beta-catenin signaling pathway. Oncotarget 2016; 7: 65374-88.
41. Christie D R, Shaikh F M, Lucas J A 4th, Lucas J A 3rd, Bellis S L. ST6Gal-I expression in ovarian cancer cells promotes an invasive phenotype by altering integrin glycosylation and function. J Ovarian Res 2008; 1:3.
42. Jung Y R, Park J J, Jin Y B, Cao Y J, Park M J, Kim E J, et al. Silencing of ST6Gal I enhances colorectal cancer metastasis by down-regulating KAI1 via exosome-mediated exportation and thereby rescues integrin signaling. Carcinogenesis 2016; 37:1089-97.
43. Zhang S, Lu J, Xu Z, Zou X, Sun X, Xu Y, et al. Differential expression of ST6GAL1 in the tumor progression of colorectal cancer. Biochem Biophys Res Commun 2017; 486:1090-6.
44. Cristofanilli M, Budd G T, Ellis M J, Stopeck A, Matera J, Miller M C, et al. Circulating tumor cells, disease progression, and survival in metastatic breast cancer. N Engl J Med 2004; 351:781-91.
45. Allard W J, Matera J, Miller M C, Repollet M, Connelly M C, Rao C, et al. Tumor cells circulate in the peripheral blood of all major carcinomas but not in healthy subjects or patients with nonmalignant diseases. Clin Cancer Res 2004; 10:6897-904.
46. Shajahan A, Supekar N T, Chapla D, Heiss C, Moremen K W, Azadi P. Simplifying glycan profiling through a high-throughput micropermethylation strategy. SLAS Technol 2020; 25:367-79.
47. Dobin A, Davis C A, Schlesinger F, Drenkow J, Zaleski C, Jha S, et al. STAR: ultrafast universal RNA-seq aligner. Bioinformatics 2013; 29: 15-21.
48. Love M I, Huber W, Anders S. Moderated estimation of fold change and dispersion for RNA-seq data with DESeq2. Genome Biol 2014; 15:550.
49. Anders S, Pyl P T, Huber W. HTSeq-a Python framework to work with high-throughput sequencing data. Bioinformatics 2015; 31:166-9.
50. Tripathi S, Pohl M O, Zhou Y, Rodriguez-Frandsen A, Wang G, Stein D A, et al. Meta- and orthogonal integration of influenza “OMICs” data defines a role for UBR4 in virus budding. Cell Host Microbe 2015; 18:723-35.
51. Muller W A, Luscinskas F W. Assays of transendothelial migration in vitro. Methods Enzymol 2008; 443:155-76.

What is claimed:

1. A method for reducing metastasis of a cancer in a subject, the method comprising administering to the subject:
a therapeutically effective amount of an anti-podocalyxin-like protein 1 (PODXL) neutralizing antibody or an antigen binding fragment thereof; and
one or more anti-cancer agents.

2. The method of claim 1, wherein the anti-PODXL neutralizing antibody or antigen binding fragment thereof is administered intravenously.

3. The method of claim 1, wherein the anti-PODXL neutralizing antibody or antigen binding fragment thereof is at a concentration of between about 5 and about 20 μg/ml.

4. The method of claim 1, wherein the one or more anti-cancer agents comprises one or more chemotherapeutic agents.

5. The method of claim 4, wherein the one or more chemotherapeutic agents comprise at least one of paclitaxel, nano-albumin-bound paclitaxel (PAX-NAB), and doxorubicin.

6. The method of claim 5, wherein the one or more chemotherapeutic agents comprise at least one of paclitaxel and nano-albumin-bound paclitaxel (PAX-NAB).

7. The method of claim 1, wherein the one or more anti-cancer agents comprises one or more CDK4/6 inhibitors.

8. The method of claim 7, wherein the one or more CDK4/6 inhibitors comprises palbociclib.

9. The method of claim 1, wherein the one or more anti-cancer agents comprises one or more immunotherapeutic agents.

10. The method of claim 9, wherein the one or more immunotherapeutic agents comprise at least one of a PD1 inhibitor and a PDL1 inhibitor.

11. The method of claim 1, wherein the one or more anti-cancer agents are administered before the anti-PODXL neutralizing antibody or antigen binding fragment thereof is administered.

12. The method of claim 1, wherein the one or more anti-cancer agents are administered concurrently with the anti-PODXL neutralizing antibody or antigen binding fragment thereof.

13. The method of claim 1, wherein the one or more anti-cancer agents are administered after the anti-PODXL neutralizing antibody or antigen binding fragment thereof is administered.

14. The method of claim 1, wherein the cancer is a breast cancer.

15. The method of claim 14, wherein the cancer is a triple negative breast cancer.

16. A method for reducing metastasis of a cancer in a subject, the method comprising administering to the subject a therapeutically effective amount of an anti-podocalyxin-like protein 1 (PODXL) neutralizing antibody or an antigen binding fragment thereof.

17. The method of claim 16, wherein the subject has been or will be administered an anticancer agent.

18. The method of claim 17, wherein the one or more anti-cancer agents comprises one or more chemotherapeutic agents.

19. The method of claim 18, wherein the one or more chemotherapeutic agents comprise at least one of paclitaxel, nano-albumin-bound paclitaxel (PAX-NAB), and doxorubicin.

20. A method for reducing circulating tumor cell cluster formation in a subject in need thereof, the method comprising administering to the subject a therapeutically effective amount of an anti-podocalyxin-like protein 1 (PODXL) neutralizing antibody or an antigen binding fragment thereof.


SEQUENCE LISTING (3 sequences)

SEQ ID NO: 1 - RNA, 25 nt, synthetic construct
  source 1..25: mol_type = other RNA
  modified_base 6: mod_base = OTHER; note = Thymidine
  modified_base 9: mod_base = OTHER; note = Thymidine
caccgtcctacaaggggccgggacc

SEQ ID NO: 2 - RNA, 25 nt, synthetic construct
  source 1..25: mol_type = other RNA
  modified_base 8..9: mod_base = OTHER; note = Thymidine
  modified_base 14: mod_base = OTHER; note = Thymidine
  modified_base 17: mod_base = OTHER; note = Thymidine
  modified_base 19: mod_base = OTHER; note = Thymidine
caccgcattcacgtggtctcgaagg

SEQ ID NO: 3 - protein, 742 aa, synthetic construct
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV